(19) United States
(12) Patent Application Publication
(10) Pub. No.: US 2026/0232831 A1
(43) Pub. Date: Aug. 13, 2026

(54) MULTIPLE FORMATS OF MOLECULAR COMPLEXES

(71) Applicant: FrontAim Biomedicines, Inc., Princeton, NJ (US)

(72) Inventor: Donglin Liu, Princeton, NJ (US)

(21) Appl. No.: 18/690,159

(22) PCT Filed: Sep. 27, 2022
(86) PCT No.: PCT/US2022/077060

§ 371 Date: Mar. 7, 2024

Related U.S. Application Data
(60) Provisional application No. 63/249,065, filed on Sep. 28, 2021.

(51) Int. Cl. A61K 47/68; A61K 51/10; A61P 35/00; C07K 16/30

(52) U.S. Cl. CPC A61K 47/6851; A61K 47/6807; A61K 47/6811; A61K 47/6813; A61K 47/6815; A61K 51/1045; A61P 35/00; C07K 16/30

(57) ABSTRACT

The present invention relates to multispecific molecule complexes. Particularly, this disclosure relates to multispecific protein complexes, such as multispecific antibodies, platform for making different formats of such complexes, and related uses. Multi-specific protein complexes, in which two or more target-specific moieties are engineered into a single molecule or complex, have expanded rapidly in recent years and offer an attractive solution for broad range of clinical and diagnostics applications. For example, bispecific antibodies and fusion proteins have been developed for binding to more than one antigen or to more than one epitope on the same antigen.

### CROSS-REFERENCE TO RELATED APPLICATIONS

This application is the U.S. national phase of International Patent Application No. PCT/US2022/077060, filed on Sep. 27, 2022, which claims priority under 35 U.S.C. § 119(e) to U.S. Provisional Patent Application No. 63/249,065, filed Sep. 28, 2021. The foregoing applications are incorporated by reference herein in their entities.

### REFERENCE TO AN ELECTRONIC SEQUENCE LISTING

The contents of the electronic sequence listing (SeqListing_19091300302_03-07-2024.xml; Size: 132.4 KB; and Date of Creation: Mar. 7, 2024) is herein incorporated by reference in its entirety.

### FIELD OF THE INVENTION

This disclosure relates to multispecific molecule complexes. Particularly, this disclosure relates to multispecific protein complexes, such as multispecific antibodies, and platforms for making different formats of such complexes.

### BACKGROUND

Multi-specific protein complexes, in which two or more target-specific moieties are engineered into a single molecule or complex, have expanded rapidly in recent years and offer an attractive solution for broad range of clinical and diagnostic applications. For example, bispecific antibodies and fusion proteins have been developed for binding to more than one antigen or to more than one epitope on the same antigen. Due to their versatile, more precise targeting abilities and higher potency as compared with conventional antibodies, multispecific antibodies have shown potential in treating various disorders, for example, as mediators to retarget effector mechanisms to disease-associated sites and become attractive for next generation antibody therapeutics. One of the major obstacles in the development of multispecific antibodies has been the difficulty of producing the materials in sufficient quality and quantity by traditional technologies, such as the hybrid hybridoma and chemical conjugation methods. Although alternative bispecific antibody platforms such as Knob-in-Hole become available, they also face challenges ranging from decreased antibody activity to poor stability and difficulties in purification or removal of impurities. The intrinsic complexity of multispecific recombinant protein molecules and antibodies can create process development and manufacturing challenges from both cost-efficiency and quality control standpoints.

Among various FDA approved agents, the Bi-specific T-cell engager (BiTE) Blinatumomab represents a unique therapeutic perspective due to its engineered structure and the clinical efficacy for relapsed or refractory B lineage leukemia or lymphoma. However, BiTE bispecific antibodies as represented by Blinatumomab have a short half-life in the body and require continuous administration for a long time, which brings great inconvenience to treatment. Such antibodies can also cause cytokine release syndrome (CRS), a collection of symptoms that can develop as a side effect of certain types of immunotherapies (Klinger M, Blood 2012119: 6226-33.), but not all bispecific formats necessarily have the same risk. A trivalent bispecific format, (X)-3s, has been generated where an anti-CD3 scFv covalently linked to a stabilized dimer of a cancer-targeting Fab using the Dock-and-Lock method. In comparison with BiTE, the (X)-3s format is a considerably less potent inducer of cytokine release, and even the addition of interferon-α to a therapeutic regimen is not likely to increase this risk (Rossi E A, Mol Cancer Ther. 2014 October; 13(10):2341-51.). However, like BiTE, the (X)-3s can be of short-life in circulation due to the lack of Fc domain, a region binding to the neonatal Fc receptor and mediating antibody recycling to the plasma membrane and subsequent release back into the serum. The half-life of IgG molecules is significantly extended by this mechanism. Thus, there is a need for methods and compositions to generate novel immunoglobulin-based formats of multispecific complexes and related platforms.

### SUMMARY

This disclosure addresses the need mentioned above in a number of aspects.

In one aspect, the disclosure provides a protein complex comprising a first moiety and second moiety. The first moiety comprises two immunoglobulin light chains and two immunoglobulin heavy chains, wherein either the two light chains or the two heavy chains are linked to two dimerization/docking domain (DDD) moieties respectively. The second moiety comprises (i) an anchoring domain (AD) moiety and (ii) an agent linked to the AD moiety. The two DDD moieties form a dimer that binds to the AD moiety. The immunoglobulin heavy chains can include a Fc region or a segment thereof. Exemplary sequences of the AD moiety include SEQ ID NO: 14 and others disclosed herein. Exemplary sequences of the DDD moiety include SEQ ID NOs: 16, 18, 20, 22, and others disclosed herein.

In one embodiment, the first moiety is a targeting moiety that specifically binds to an antigen or epitope, and the second moiety is an effector moiety. In that case, the above-mentioned agent is an effector agent.

In another embodiment, the first moiety is an effector moiety comprising an effector agent, while the second moiety is a targeting moiety that specifically binds to an antigen or epitope. In that case, the above-mentioned agent can be a targeting agent that specifically binds to the antigen or epitope.

In yet another embodiment, the first moiety and the second moiety are two targeting moieties that specifically bind to two antigens or epitopes. The two antigens or epitopes can be the same or different.

In a further embodiment, the first moiety and the second moiety are two effector moieties, and the agents are effector agents. The two effector agents can be the same or different.

In a second aspect, the disclosure provides a fusion protein comprising (i) a dimerization/docking domain (DDD) moiety and (ii) an immunoglobulin light chain fused to the DDD moiety, or an immunoglobulin heavy chain fused to the DDD moiety. The immunoglobulin heavy chains can include a Fc region or a segment thereof.

In the above-described protein complex or fusion protein, the DDD or the AD moiety may be fused at any suitable positions (e.g., the N-terminus, the C-terminus, or the middle) of a polypeptide chain.

In some embodiments, each DDD moiety can be inserted in each immunoglobulin heavy chain. For example, the DDD moiety may be inserted in a hinge region, a variant hinge region, or a hybrid hinge region of the immunoglobulin heavy chain or a hinge flank region thereof. In particular, the DDD moiety may be inserted below or above the middle hinge (CPPCP, SEQ ID NO: 41) of IgG1, below or above the middle hinge (CCVECPPCP, SEQ ID NO: 42) of IgG2, below or above the partial middle hinge (CPRCP, SEQ ID NO: 43) of IgG3, below or above the middle hinge (CPSCP, SEQ ID NO: 46) of IgG4. The DDD moiety may also be inserted in the middle hinge of IgG1, IgG2, IgG3, and IgG4, or lower hinge (APELLGG, SEQ ID NO: 47) of IgG1, IgG3, and IgG4, or lower hinge (APPVAG, SEQ ID NO: 48) of IgG2. The DDD moiety may also be inserted in the N-terminal region (P238 to C261, SEQ ID NOs: 49, 50, 51, 52) of the IgG CH2 domain. Further, the DDD moiety may also be inserted in the hinge region or a flank region of the IgA1 or IgA2 heavy chain (SEQ ID NOs: 53, 54).

In others, each DDD moiety may be fused to the C-terminus of each immunoglobulin light chain. In some examples, the DDD moiety may be fused to the C-terminus of the immunoglobulin light chain via a linker sequence. The linker sequence may comprise at least one cysteine and the protein complex may comprise a disulfide bond between two linker sequences. The disulfide bond between two linker sequences helps form a stably tethered structure. Examples of the linker include SEQ ID NOs: 11 and 13, or those fused in SEQ ID NOs: 18 and 19 and others disclosed hereon.

In the above-described protein complex or fusion protein, each DDD moiety may be fused to the C-terminus of each immunoglobulin heavy chain.

The targeting moiety in the above-described protein complex may bind specifically to a tumor associated antigen or a disease associated antigen. Examples of the tumor associated antigen or the disease associated antigen include, but not limited to, Trop2, EpCAN, GPRC5, FcR-5, ROR1, BCMA, CD15, CD16, CD19, CD20, CD22, CD27, CD30, CD33, CD40, CD47, CD40L, CD66, CD70, CD74, CD79b, CD80, CD95, CD133, CD160, CD166, CD229, MUC1, MUC5, MUC16, IGF-1R, EGFR, HER2, HER3, EGP2, HLA-DR, TNF-α, TRAIL receptor, ICOS, ICOSL, VEGFR, hypoxia inducible factor (HIF), Flt-3, folate receptor, TDGF1, TfR, Mesothelin, PSMA, CEACAM5, CEACAM6, B7, TFN-α, IFN-β, IFN-γ, TFN-ζ, IL-1β, IL2, IL6, IL-6R, IL-15, IL-15R, IL-17, IL-17R, IL-12, C1r, C1s, C2, C3, C5, C5a, C5aR1, C6, MASPs, MSAP2, MASP3, FB, FD, Properdin, Lag-3, CTLA-4, PD-1, PD-L1, TIM3, SIRPα, TIGIT, OX40, OX40L, 4-1BB, NKG2A, NKG2B, BTLA, GITR, GITRL, TCR, Nectin-4, c-Met, LIV1, Mesothelin, DLL3, DLL4, Tissue factor, TGE-β, TGF-β receptor, CLDN18.2, or DKK1.

In some embodiments of the protein complex, the immunoglobulin light chain may comprise a light chain variable region comprising LCDR1, LCDR2 and LCDR3, wherein the LCDR1, LCDR2 and LCDR3 comprise the respective sequences of SEQ ID NOs: 27-29. The immunoglobulin heavy chain may comprise a heavy chain variable region comprising HCDR1, HCDR2 and HCDR3, wherein the HCDR1, HCDR2 and HCDR3 comprise the respective sequences of SEQ ID NOs: 30-32. The immunoglobulin light chain may comprise the sequence of SEQ ID NO: 17 or 19. The immunoglobulin heavy chain may comprise the sequence of SEQ ID NO: 21 or 23 or 24. In one example, the immunoglobulin light chain comprises the sequence of SEQ ID NO: 26, and/or the immunoglobulin heavy chain comprises the sequence of SEQ ID NO: 25.

In the protein complex, the effector agent may comprise an antibody or an antigen-binding fragment thereof, aptamers, a ligand, a cytotoxin, a chemotherapeutic agent, a detectable label or tag, a drug, a pro-drug, a toxin, an enzyme, an immunomodulator, a checkpoint inhibitor, an anti-angiogenic agent, a pro-apoptotic agent, a cytokine, a growth factor, a hormone, a cytokine, a radioisotope, a protein, a peptide, a peptide mimetic, a polynucleotide, a RNAi oligosaccharide, a natural or synthetic polymeric substance, a nanoparticle, a quantum dot, an organic compound, or an inorganic compound. The antibody may bind specifically to a marker on immune cells. In one embodiment, the antibody binds specifically to a T cell specific marker, such as CD3. The antibody may be a single chain antibody. The antibody may comprise the sequence of 3scFv, which may include one or more or all of the sequences of SEQ ID NOs: 10, 12, or 33. The single chain antibody may include a leader sequence, such as that of SEQ ID NO: 9, or a tag sequence, such as that of SEQ ID NO: 15.

In a third aspect, the disclosure features an antibody or antigen-binding fragment thereof that specifically binds to human Trop2, comprising: (i) a light chain or a light chain variable region that comprises LCDR1, LCDR2 and LCDR3 comprising the respective sequences of SEQ ID NOs: 27-29, and/or (ii) a heavy chain or a heavy chain variable region that comprises HCDR1, HCDR2, and HCDR3 comprising the respective sequences of SEQ ID NOs: 30-32. In one embodiment, the light chain variable region comprises the sequence of SEQ ID NO: 1 or 5, and/or the heavy chain variable region comprises the sequence of SEQ ID NO: 3 or 7.

The protein complex or fusion protein or antibody or antigen-binding fragment described above may further comprise a Fc region or a variant Fc constant region, e.g., in a heavy chain.

In another aspect, the disclosure provides an isolated nucleic acid or a set of nucleic acids encoding the protein complex, or fusion protein, or one or more of the CDRs, or the heavy chain variable region, or the light chain variable region, or antibody, or antigen-binding fragment described above. Examples of the sequences include SEQ ID NOs: 2, 4, 6, and 8. The nucleic acid or nucleic acids can be used to express a polypeptide or polypeptides having one or more of the HCDRs or LCDRS, a chain of the antibody or antigen-binding fragment, or the antibody or fragment, or fusion protein, or protein complex described above. For this purpose, one can operatively link the nucleic acid or nucleic acids to suitable regulatory sequences to generate an expression vector or vectors.

Accordingly, within the scope of this disclosure are an expression vector(s), and a host cell comprising the vector(s) or nucleic acid(s), and a method for producing the protein complex, or the fusion protein, or one or more of the CDRs, or the heavy chain variable region, or the light chain variable region, or the antibody, or the antigen-binding fragment described above.

The method may include obtaining a cultured host cell comprising a nucleic acid sequence or nucleic acid sequences encoding the protein complex or fusion protein or antibody or antigen-binding fragment; culturing the cell in a medium under conditions permitting expression of the fusion protein or protein complex and assembling of the protein complex, an antibody, or fragment thereof inside the cell or outside the cells, and purifying the protein complex or fusion protein or antibody or antigen-binding fragment from the cultured cell or the medium of the cell. In a preferred embodiment, the assembling is intracellular.

The disclosure further provides a pharmaceutical composition comprising the protein complex or fusion protein or antibody or antigen-binding fragment described above and a pharmaceutically acceptable carrier.

Also provided is a method for treating a cancer or a disease in a subject in need thereof. The method comprises administering to the subject an effective amount of the protein complex or fusion protein or antibody or antigen-binding fragment with pharmaceutical composition described above. Examples of the disease include a cancer disease (e.g., breast cancer, lung cancer, gastric cancer, colorectal cancer, bladder cancer, liver cancer, prostate cancer, pancreatic cancer, melanoma, leukemia, lymphoma, multiple myeloma) an immunological disease (e.g., autoimmune diseases) and an infection with a pathogen (such as a virus, a bacterium, a fungus, or parasite).

The details of one or more embodiments of the disclosure are set forth in the description below. Other features, objectives, and advantages of the disclosure will be apparent from the description and from the claims.

### BRIEF DESCRIPTION OF THE DRAWINGS

FIGS. 1A, 1B, 1C, and 1D are set of diagrams showing four schematic models or formats of bispecific antibodies. (FIG. 1A) and (FIG. 1B), an anti-CD3 scFv (3scFv) and an anti-tumor associated antigen (anti-TAA) IgG are site-specifically assembled via the C-terminus-fused AD2 in anti-CD3 scFv and DDD2 in two light chains of anti-TAA IgG respectively. The intramolecular DDD2 dimer is formed and stabilized with the addition of a disulfide bond between two linkers in model B. (FIG. 1C) AD2 of anti-CD3 scFv is conjugated to the DDD2 dimer inserted into the hinge region of anti-TAA IgG. (FIG. 1D) two DDD2 peptides fused to the C-termini of heavy chains of anti-TAA IgG are dimerized and conjugated with AD2 of anti-CD3 scFv.

FIGS. 2A, 2B, 2C, 2D, and 2E are diagrams showing the variable region sequences of antibody L0125 against human Trop2. (FIG. 2A) The variable region of a L0125 light chain. (FIG. 2B) The variable region of a L0125 heavy chain, (FIG. 2C) The amino acid sequences of L0125 LCDR1, LCDR2 and LCDR3 (SEQ ID NOs: 27-29) and HCDR1, HCDR2 and HCDR3 (SEQ ID NOs: 30-32). (FIG. 2D). The variable region of a humanized L0125 (hL0125) light chain, (FIG. 2E) The variable region of a hL0125 heavy chain,

FIG. 3 is a chart showing binding of cL0125 and hL0125 antibodies to A-431 cells. A-431 cells were dissociated from culture, dispensed into a 96-well plate at 2×10⁵/well, and incubated with indicated agents at 4° C. for 45 min. After wash with PBS, the cells were incubated with AF488 labeled goat anti-human IgG Fc. Binding was analyzed by flow cytometry using Attune N×T Flow Cytometer.

FIGS. 4A, 4B, 4C, and 4D are photographs showing bispecific antibodies and their modules in SDS-PAGE gel. Four formats of bispecific antibodies against CD3 (3scFv) and Trop2 (hL0125) were constructed and produced as designed in previous models A-D in FIG. 1. (FIG. 4A) 3scFv×hL0125A; (FIG. 4B) 3scFv×hL0125B; (FIG. 4C) 3scFv×hL0125C; and (FIG. 4D) 3scFv×hL0125D. Lanes: M, protein ladder; 1 and 4, 3scFv-AD2; 2 and 5, hL0125-DDD2; 3 and 6, bispecific conjugates of 3scFv-AD2 and hL0125-DDD2. R, reducing; NR, non-reducing.

FIGS. 5A and 5B are charts showing binding of bispecific antibodies 3scFv×hL0125C to (FIG. 5A) cell surface CD3 of Jurkat and (FIG. 5B) Trop2 of ME-180. For CD3 binding, Jurkat cells were dispensed into a 96-well plate at 2×10⁵/well, and incubated with indicated agents at 4° C. for 45 min. After wash with PBS, cells were incubated with AF488 labeled goat anti-human IgG Fe. For Trop2 binding, ME-180 cells were dissociated from culture, dispensed into a 96-well plate at 2×10⁵/well, and incubated with indicated agents at 4° C. for 45 min. After wash with PBS, cells were incubated with AF488 labeled goat anti-human IgG Fc. Binding was analyzed by low cytometry using Attune N×T Flow Cytometer.

FIGS. 6A, 6B, 6C, and 6D are charts showing in vitro cytotoxicity of 3scFv×hL0125C. FIG. 6A shows cytotoxicity on MDA-MB-468 cells. FIG. 6B shows cytotoxicity on MDA-MB-231 cells. FIG. 6C shows cytotoxicity on BT-474 cells. FIG. 6D shows cytotoxicity on HCT 116 cells. Cancer Cells were combined with human PBMCs and dispensed into 96-well plates at 200 μl/well to provide 1×10⁴ tumor cells and 1×10⁵ PBMC cells (PBMC-to-target ratio of 10:1) in each well. Bispecific Ab (3scFv×hL0125C) and its monospecific modules starting at 20 nmol/L, were 4-fold serially diluted to treat the mixed cells. After 60 h incubation, media were removed and replaced with fresh media to flush PBMCs and dead cancer cells. Cell viabilities were measured with MTS reagent. The assay was done in triplicates.

FIG. 7 is a set of photographs showing effects of 3scFv×hL0125C on tumorigenesis. HCT 116 cells were combined with human PBMCs and dispensed into 96-well spheroid microplates at 200 μL/well to provide 200 tumor cells and 1,600 PBMC cells (PBMC-to-target ratio of 8:1) in each well. Bispecific Ab (3scFv×hL0125C) and its monospecific modules starting at 20 nmol/L, were 4-fold serially diluted to treat the mixed cells. After 7-day incubation, tumor image in each well was captured using BVOS FL Auto 2. The assay was done in duplicates.

FIG. 8 is a chart showing stability of bispecific antibody 3scFv×hL0125C. Sample was diluted to 100 μg/ml in human plasm and incubated at 37° C. for 7 days. Afterwards, the stability was evaluated by cell surface binding and detection. Specifically, sample was 2-fold diluted in PBS and incubated with MDA-MB-468 cells for binding to cell surface Trop2 at 4° C. for 45 min, then detected by anti-His-AF488 (the anti-CD3-AD2 module was His-tagged at C-terminus). The fresh control sample was diluted to 100 μg/ml in human plasm and immediately analyzed for similar binding assay.

FIGS. 9A and 9B are charts showing binding of bispecific antibodies 3scFv×hL0125B to (FIG. 9A) cell surface CD3 of Jurkat and (FIG. 9B) Trop2 of MDA-MB-468. For CD3 binding, Jurkat cells were dispensed into a 96-well plate at 2×10⁵/well, and incubated with indicated agents at 4° C. for 45 min. After wash with PBS, cells were incubated with AF488 labeled goat anti-human IgG Fc. For Trop2 binding, MDA-MB-468 cells were dissociated from culture, dispensed into a 96-well plate at 2×10⁵/well, and incubated with indicated agents at 4° C. for 45 min. After wash with PBS, cells were incubated with AF488 labeled goat anti-human IgG Fe. Binding was analyzed by flow cytometry using Attune N×T Flow Cytometer.

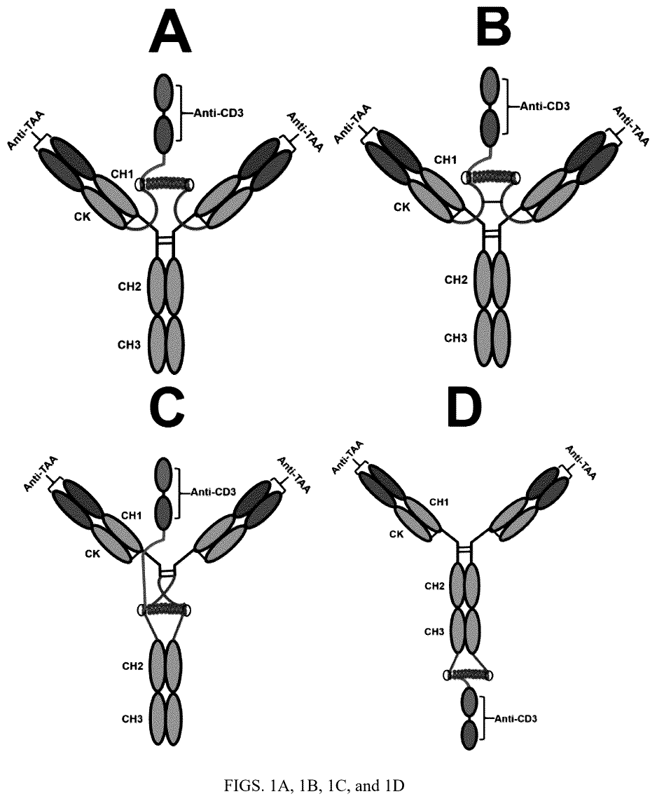

FIGS. 1A, 108, and 10C are charts showing in vitro cytotoxicity of 3scFv×hL0125B. FIG. 10A shows cytotoxicity on MDA-MB-468 cells. FIG. 10B shows cytotoxicity on BT-474 cells. FIG. 10C shows cytotoxicity on ME-180 cells. Cancer cells were combined with human PBMCs and dispensed to 96-well plate at 200 μl/well to provide 1×10⁴ tumor cells and 1×10⁵ PBMC cells (PBMC-to-target ratio of 10:1) in each well. Bispecific Ab (FABM12×hL0125) or its monospecific module starting at 20 nmol/L, was 4-fold serially diluted to treat the mixed cell. After 60 h incubation, media were removed and replaced with fresh media to flush PBMCs and dead cancer cells. Cell viabilities were measured with MTS reagent. The assay was done in triplicates.

FIG. 11 is a chart showing stability of bispecific antibodies 3scFv×hL0125B. A sample was diluted to 100 μg/ml in human plasm and incubated at 37° C. for 7 days. Afterwards, the stability was evaluated by cell surface binding and detection. Specifically, sample was 2-fold diluted in PBS and incubated with MDA-MB-468 cells for binding to cell surface Trop2 at 4° C. for 45 min, then detected by anti-His-AF488 (the anti-CD3-AD2 module was His-tagged at C-terminus). The control sample was diluted to 100 μg/ml in human plasm and immediately analyzed for similar binding assay.

FIGS. 12A and 122B are photographs showing bispecific antibodies in SDS-PAGE gel. FIG. 12A shows 3scFv×hL0125Cm in SDS-PAGE gel, FIG. 12B shows 3scFv×hACD20Cm in SDS-PAGE gel. Two bispecific antibodies were originally constructed as designed in the model C of FIG. 1: 3scFv×hL0125Cm against CD3 and Trop2, 3scFv×hACD20Cm against CD3 and CD20, respectively. To eliminate off-target T cell activation by FcγR-expressing cells, these bispecific antibodies were further engineered to produce the Cm model that lacks FcR binding. Lanes: M, protein ladder; 1 and 4, 3scFv-AD2; 2 and 5, hL0125-DDD2 or hACD20-DDD2; 3 and 6, bispecific conjugates of 3scFv×hL0125Cm or 3scFv×hACD20Cm. R, reducing; NR, non-reducing.

### DETAILED DESCRIPTION OF THE INVENTION

This disclosure relates to multispecific molecule complexes, such as multispecific protein complexes, e.g., multispecific or bispecific antibodies, and platforms for making different formats of such complexes. Certain aspects of this invention are based, at least in part, on unexpected discoveries that heterologous protein-protein interaction domains (e.g., DDD and AD) can be incorporated or linked to the proteins or antibodies at various unexpected locations to generate functional multispecific protein complexes or multispecific antibodies.

As compared to conventional bispecific antibody platforms, the platform disclosed herein represents a unique novel design, which not only differs from the conventional heavy chain heteromerization bispecific antibody, but also does not require the use of CrossMab-like domain recombination. This design is not a simple protein fusion expression. Rather, due to its significant optimization in structure and biological activity, the multispecific platform provides many advantages over the convention platforms including ease in production and purification Indeed, as disclosed herein, multispecific or bispecific molecules can be expressed and assembled in a single cell and as such conventional standard IgG isolation and purification process can be applied to obtain the multispecific or bispecific molecules.

In certain embodiments, the platform allows one to retain intact IgG, Fc, and/or Fc molecule domain structures and achieve a “1+2” valence mode against two different targets. In the context of immune-oncology, this allows targeting cancer cells and redirecting T cells in close contact. This “1+2” mode can meet the requirements of different affinities for two kinds of cells or two targets.

### Multispecific Molecule Complexes

One aspect of this disclosure provides a platform of multispecific protein complexes. In one embodiment, the protein complex in general can have, among others, two functional components or moieties: (1) a first moiety comprising two immunoglobulin light chains and two immunoglobulin heavy chains, wherein either the two light chains or the two heavy chains are linked to two dimerization/docking domain (DDD) moieties respectively, and (2) a second moiety comprising (i) an anchoring domain (AD) moiety and (ii) an agent linked to the AD moiety. The two copies of DDD moieties form a dimer that binds to the AD moiety. Preferably, one of the two immunoglobulin heavy chains can have a Fc region or a fragment thereof. In some embodiments, both of the two immunoglobulin heavy chains can have Fc regions or Fc fragments.

Either the first moiety or the second moiety can comprise or be a targeting moiety or an effector moiety. For example, in one embodiment, the first moiety is a targeting moiety that specifically binds to an antigen or epitope, and the second moiety is an effector moiety and the agent is an effector agent. In another embodiment, the first moiety is an effector moiety comprising an effector agent and the second moiety is a targeting moiety and the agent is a targeting agent that specifically binds to an antigen or epitope. In a further embodiment, the first moiety and the second moiety are two targeting moieties that specifically bind to two antigens or epitopes. In yet another embodiment, the first moiety and the second moiety are two effector moieties, and the agents are effector agents.

Together, the first moiety and the second moiety can provide multiple binding sites and the close proximity of the binding sites can lead to the formation of new complexes (of a target cell and an effector agent) and trigger new cellular contacts. For example, as disclosed herein, with two or more sites for interaction with target cells (e.g., cancer cells), more targeted binding can be achieved and additional immune responses can be activated that involve immune effector cells (e.g., T-Cells and natural killer cells) leading to greater targeted cytotoxic effects.

### Immunoglobulin

Immunoglobulin refers to a naturally occurred or recombinantly produced antibody molecule that acts as a critical part of the immune response by specifically recognizing and binding to antigens. There are five major immunoglobulin classes: IgA, IgD, IgE, IgG and IgM. IgG and IgA are further grouped into subclasses (e.g., in human IgG1, IgG2, IgG3, IgG4, IgA1 and IgA2) based on additional small differences in the amino acid sequences of heavy chain. The various immunoglobulin classes and subclasses differ in their biological features, structure, target specificity.

Immunoglobulins are heterodimeric proteins composed of two heavy and two light chains, where the light chain can consist of either a κ or a λ, chain. Both heavy chains or light chains can be separated functionally into variable domains (Fv) that binds antigens and constant domains (Fc) that specify effector functions such as activation of complement or binding to Fc receptors. The light chains contain only one constant domain (Ck or Cλ), whereas heavy chains often contain three such domains (CH1, CH2, CH3) and a hinge region between the first (CH1) and second (CH2) domains (Schroeder et al, J Allergy Clin Immunol. 2010, 125(2 02): S41-S52).

A “hinge”, “hinge domain” or “hinge region” or “antibody hinge region” refers to the domain of a heavy chain constant region that joins the CH1 domain to the CH2 domain and includes the upper, middle, and lower portions of the hinge (Roux et al., 1998 J Immunol 161:4083). The hinge provides varying levels of flexibility between the binding and effector regions of an antibody and provides sites for intermolecular disulfide bonding between the two heavy chain constant regions. As used herein, a hinge starts at E216 and ends at Gly237 for all IgG isotypes (Roux et at, 1998 J Immunol 161:4083). The sequences of wild type IgG1, IgG2, IgG3 and IgG4 hinges are shown in Table A, The term “hinge” includes wild type hinges (such as those set forth in Tables 2, 3 and 4), variant hinges as well as hybrid hinges thereof (e.g., non-naturally occurring hinges or modified hinges). For example, the term “IgG1 hinge” includes wild type IgG1 hinge (E216-G237, SEQ ID NO. 49), as shown in Table B. and variants having 1, 2, 3, 4, 5, 1-3, 1-5, 3-5 and/or at most 5, 4, 3, 2, or 1 mutation(s), e.g., substitutions, deletions, or additions. In certain embodiments, a hinge is a hybrid hinge that comprises sequences from at least two isotypes. For example, a hinge may comprise the upper, middle, or lower hinge from one isotype and the remainder of the hinge from one or more other isotypes. For example, a hinge can be an IgG2/IgG1 hinge, and may comprise, e.g., the upper and middle hinges of IgG2 and the lower hinge of IgG1. A hinge may have effector function or be deprived of effector function. For example, the lower hinge of wild type IgG1 provides effector function.

TABLE A
IgG hinge region amino acids (Roux et al., 1998 J Immunol 161:4083)

| Ig Type | C-terminal C_{H}1* | Upper Hinge | Middle Hinge | Lower Hinge |
|---|---|---|---|---|
| IgG1 | VDKRV | EPKSCDKTHT | CPPCP | APELLGG |
| | (SEQ ID NO: 34) | (SEQ ID NO: 36) | (SEQ ID NO: 41) | (SEQ ID NO: 47) |
| IgG2 | VDKTV | ERK | CCVECPPQP | APPVAG |
| | (SEQ ID NO: 36) | (SEQ ID NO: 37) | (SEQ ID NO: 42) | (SEQ ID NO: 48) |
| IgG3 | VDKRV | ELKTPLGDTTHT | CPRQP (SEQ ID NO: 43) | APELLGG |
| (17-15-15-16) | (SEQ ID NO: 34) | (SEQ ID NO: 38) | (EPKSCDTPPPCPRQP)₃ | (SEQ ID NO: 47) |
| | | | (SEQ ID NO: 44) | |
| IgG3 (17-15-15) | VDKRV | ELKTPLGDTTHT | CPRQP | APELLGG |
| | (SEQ ID NO: 34) | (SEQ ID NO: 38) | (SEQ ID NO: 43) | (SEQ ID NO: 47) |
| | | | (EPKSCDTPPPCPRQP)₂ | |
| IgG3 (17-15) | VDKRV | ELKTPLGQTTHT | CPRCP (SEQ ID NO: 43) | APELLGG |
| | (SEQ ID NO: 34) | (SEQ ID NO: 38) | (EPKSCQTPPPCPRCP)₁ | (SEQ ID NO: 47) |
| | | | (SEQ ID NO: 44) | |
| IgG3 (16-15-15) | VDKRV | EPKS | CDTPPPQPRQP | APELLGG |
| | (SEQ ID NO: 34) | (SEQ ID NO: 39) | (SEQ ID NO: 45) | (SEQ ID NO: 47) |
| | | | (EPKSCDTPPPCPRQP)₂ | |
| | | | (SEQ ID NO: 44) | |
| IgG3 (15) | VDKRV | EPKS | CDTPPPQPRQP | APELLGG |
| | (SEQ ID NO: 34) | (SEQ ID NO: 39) | (SEQ ID NO: 45) | (SEQ ID NO: 47) |
| IgG4 | VDKRV | ESKYGPP | CPSCP | APEFLGG |
| | (SEQ ID NO: 34) | (SEQ ID NO: 40) | (SEQ ID NO: 46) | (SEQ ID NO: 47) |

*C-terminal amino acid sequences of the CH1 domains.

The term “CH1 domain” refers to the heavy chain constant region linking the variable domain to the hinge in a heavy chain constant domain. As used herein, an IgG CH1 domain starts at A118 and ends at V215 (Table B), an IgA CH1 domain starts at A120 and ends at P221 (Table C). The term “CH1 domain” includes wild type CH1 domains (such as having SEQ ID NO: 49 for IgG1 and SEQ ID NO: 50 for IgG2, Table B), as well as variants thereof (e.g., non-naturally occurring CH1 domains or modified CH1 domains). For example, the term “CH1 domain” includes wild type CH1 domains and variants thereof having 1, 2, 3, 4, 5, 1-3, 1-5, 3-5 and/or at most 5, 4, 3, 2, or 1 mutation(s), e.g., substitutions, deletions, or additions. Exemplary CH1 domains include CH1 domains with mutations that modify a biological activity of an antibody, such as ADCC, CDC or half-life.

TABLE B
IgG heavy chain constant region amino acids

| CH1 | | | | |
|---|---|---|---|---|
| IgG1 | 118 | ASTKGPSVFPLAPSSKSTSGGTAALGCLVKDYFPEPVTVSWNSGALTSGVETFPAVLQSSGLYSLSSVV | | 186 (Eu Idx) |
| IgG3 | | 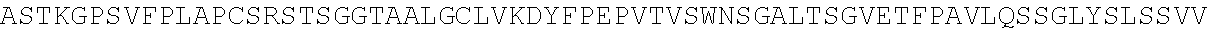 | | |
| IgG2 | | ASTKGPSVFPLAPCSRSTSESTAALGCLVKDYFPEPVTVSWNSGALTSGVETFPAVLQSSGLYSLSSVV | | |
| IgG4 | | ASTKGPSVFPLAPCSRSTSESTAALGCLVKDYFPEPVTVSWNSGALTSOVETFPAVLQSSGLYSLSSVV | | |
| IgG1 | 187 | TVPSSSLGTQTYICNVNHKPSNTKVDKKV | 215 (Bu Idx) | |
| IgG3 | | TVPSSSLGTQTYTCNVNHKPSNTKVDKRV | | |
| IgG2 | | TVPSSNFGTQTYTCNVNHKPSNTKVDKTV | | |
| IgG4 | | TVPSSSLGTKTYTCNVDHKPSNTKVDKRV | | |
| Hinge | | | | |
| IgG1 | | 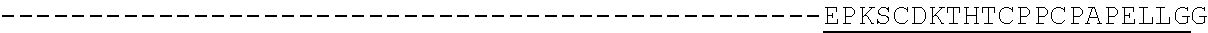 | | 237 (Bu Idx) |
| IgG3 | | 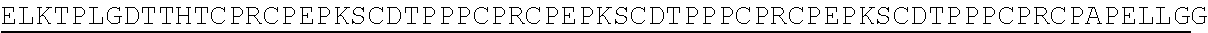 | | |
| IgG2 | | 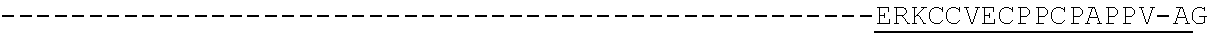 | | |
| IgG4 | | 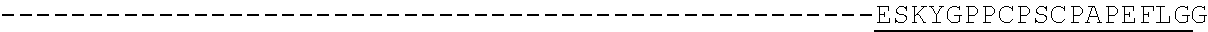 | | |
| CH2 | | | | |
| IgG1 | 238 | PSVFLFPPKPKDTLMISRTPEVTCVVVDVSHEDPEVKFNWYVDGVEVHNATKRKPREEQYNSTYRVVSVL | | 306 (Bu Idx) |
| IgG3 | | PSVFLFPPKPKDTLMISRTPEVTCVVVDVSHEDPEVQFKWYVDGVEVHNATKRKPREEQYNSTFRVVSVL | | |
| IgG2 | | PSVFLFPPKPKDTLMISRTPEVTCVVVDVSHEDPEVQFNWYVDGVEVHNATKRKPREEQFNSTFRVVSVL | | |
| IgG4 | | PSVFLFPPKPKDTLMISRTPEVTCVVVDVSQEDPEVQFNWYVDGVEVHNATKRKPREEQFNSTYRVVSVL | | |
| IgG1 | 307 | TVLHQDWLNGKEYKCKVSNKALPAPIEKISKAK | 340 (Bu Index) | |
| IgG3 | | TVLHQDWINGKEYKCKVSNKALPAPIEKISKTK | | |
| IgG2 | | TVVHQDWLNGKEYKCKVSNKGLPAPIEKISKTK | | |
| IgG4 | | TVLHQDWLNGKEYKCKVSNKGLPSSIEKISKAK | | |
| CH3 | | | | 409 (Eu Idx) |

| | | |
|---|---|---|
| IgG1 | 341 | GQPREPQVYTLPPSRDELTKNQVSLTCLVKGFYPSDIAVEWESNGQPENNYKTTPPVLDSDGSFFLYSK |
| IgG3 | | GQPREPQVYTLPPSREEMTKNQVSLTCLVKGFYPSDIAVEWESSGQPENNYNTTPPMLDSDGSFFLYSK |
| IgG2 | | GQPREPQVYTLPPSREEMTKNQVSLTCLVKGFYPSDIAVEWESNGQPENNYKTTPPMLDSDGSFFLYSK |
| IgG4 | | GQPREPQVYTLPPSQEEMTKNQVSLTCLVKGFYPSDIAVEWESNGQPENNYKTTPPVLDSDGSFFLYSR |

| | | | | |
|---|---|---|---|---|
| IgG1 | 410 | LTVDKSRWQQGNVFSCSVMHEALHNHYTQKSLSLSPGK | 447 (Eu Idx) | Sequence ID No. 49 |
| IgG3 | | LTVDKSRWQQGNIFSCSVMHEALHNRFTQKSLSLSPGK | | Sequence ID No. 51 |
| IgG2 | | LTVDKSRWQQGNVFSCSVMHEALHNHYTQKSLSLSPGK | | Sequence ID No. 50 |
| IgG4 | | LTVDKSRWQEGNVFSCSVMHEALHNHYTQKSLSLSPGK | | Sequence ID No. 52 |

The term “CH2 domain” refers to the heavy chain constant region linking the hinge to the CH3 domain in a heavy chain constant domain. As used herein, an IgG CH2 domain starts at P238 and ends at K340 (Table B), and an IgA CH2 domain starts at C241 and ends at S341 (Table C). The term “CH2 domain” includes wild type CH2 domains (such as having SEQ ID NO: 49 for IgG1; Table B), as well as variants thereof (e.g., non-naturally occurring CH2 domains or modified CH2 domains). For example, the term “CH2 domain” includes wild type CH-2 domains and variants thereof having 1, 2, 3, 4, 5, 1-3, 1-5, 3-5 and/or at most 5, 4, 3, 2, or 1 mutation(s), e.g., substitutions, deletions, or additions, Exemplary CH2 domains include CH2 domains with mutations that modify a biological activity of an antibody, such as ADCC, CDC or half-life. In certain embodiments, a CH2 domain comprises modifications that affect a biological activity of an antibody are provided herein.

The term “CH3 domain” refers to the heavy chain constant region that is C-terminal to the CH2 domain in a heavy chain constant domain. As used herein, an IgG CH3 domain starts at G341 and ends at K447 (Table B), and an IgA CH3 domain starts at G342 and ends at Y472 (Table C). The term “CH3 domain” includes wild type CH3 domains (such as having SEQ ID NO: 49 for IgG1; Table B), as well as variants thereof (e.g., non-naturally occurring CH3 domains or modified CH3 domains). For example, the term “CH3 domain” includes wild type CH3 domains and variants thereof having 1, 2, 3, 4, 5, 1-3, 1-5, 3-5 and/or at most 5, 4, 3, 2, or 1 mutation(s), e.g., substitutions, deletions, or additions. Exemplary CH3 domains include CH3 domains with mutations that modify a biological activity of an antibody, such as ADCC, CDC or half-life.

TABLE C
IgA heavy chain constant region amino acids (Patent US10822399B2)

IgA-CH1
Cα1-1 120 ASPTSPKVFPLSLCSTQPDGNVVIACLVQGFFPQEPLSVTWSESGQGVTARNFPPSQDASGD 181 (Bur)
Cα2-1 ASPTSPKVFPLSLDSTPQDGNVVVACLVQGFFPQEPLSVTWSESGQNVTARNFPPSQDASGD
Cα1-1 182 LYTTSSQLTLPATQCLAGKSVTCHVKHYTNPSQDVTVPCP 221 (Bur)
Cα2-1 LYTTSSQLTLPATQCPDGKSVTCHVKHYTNPSQDVTVPCP
Hinge
Cα1-1 222 VPSTPPTPSPSTPPTPSPS 240(Bur)
Cα2-1 VPPPPP-------------
IgA-CH2
Cα1-2 241 CCHPRLSLHRPALEDLLLGSEANLTCTLTGLRDASGVTFTWTPSSGKSAVQG 292 (Bur)
Cα2-2 CCHPRLSLHRPALEDLLLGSEANLTCTLTGLRDASGATFTWTPSSGKSAVQG
Cα1-2 293 PPERDLCGCYSVSSVLPGCAEPWNHGKTFTCTAAYPESKTPLTATLSKS 341 (Bar)
Cα2-2 PPERDLCGCYSVSSVLPGCAQPWNHGETFTCTAAHPELKTPLTANITKS
IgA-CH3
Cα1-3 342 GNTFRPEVHLLPPPSEELALNELVTLTCLARGFSPKDVLVRWLQGSQELPREKYLTWASRQEPS 405 (Bur)
Cα2-3 GNTFRPEVHLLPPPSEELALNELVTLTCLARGFSPKDVLVRWLQGSQELPREKYLTWASRQEPS
Cα1-3 406 QGTTTFAVTSILRVAAEDWKKGDTFSCMVGHEALPLAFTQKTIDRLAGKPTHVNVSVVMAEVDGTCY
472 (Bur)
Cα2-3 QGTTTFAVTSILRVAAEDWKKGDTFSCMVGHEALPLAFTQKTIDRMAGKPTHVNVSVVMAEVDGTCY
(Cα1: Sequence ID No. 53; Co2: Sequence ID No. 54)

Amino acid numbering above human IgA is according to the commonly adopted scheme used for IgA1 Bur (Liu et al, Science, 1976, Vol. 193: 1017-1020).

### Targeting Moiety

One component of the above-described protein complex can be a targeting moiety that binds specifically to a target. A targeting moiety or targeting domain or targeting agent, used interchangeably herein, refers to an entity (e.g., a molecule) that promotes the interaction, e.g., binding of a protein with a target, and/or directs a protein to a target. A targeting moiety or domain or agent can be a polypeptide, an antibody, or an antigen-binding portion thereof.

As used herein, a “target” can be a cell, a pathogen, a metabolite, a polypeptide complex, or any molecule or structure that resides in a tissue or circulates in the circulatory system or lymphatic system of a subject, such as an immune cell or a cancer cell. A target can be any of such aspects which readily interacts with targeting moiety or targeting domain. In some embodiments, the term refers to a moiety. e.g., an antibody molecule, that as a component of a therapeutic compound, localizes the therapeutic compound preferentially to a target tissue or tart cell.

In some embodiments, the targeting moiety can function in the above-described protein complex by delivering the protein complex and/or the effector moiety to the local environment of pathogens, disease cells, or cancer cells, enabling a localized treatment strategy. In certain embodiments, the targeting moiety targets the cancer cells by specifically binding to the pathogens, disease cells, or cancer cells. As disclosed herein, the above described targeting moiety or targeting domain or targeting agent can specifically bind to a disease-associated antigen, such as a tumor-associated antigen.

A “disease-associated antigen” refers to antigen that is expressed coincidentally with a particular disease process, where antigen expression correlates with or predicts development of that disease. A disease-associated antigen can be an antigen recognized by T-cells or B-cells. Some disease-associated antigens may also be tissue-specific. A tissue-specific antigen is expressed in a limited number of tissues. Disease-associated antigens can be, for example, tumor-associated antigens, viral antigens, bacterial antigens, fungal antigens, or parasite antigens.

A “tumor-associated antigen” refers to an antigen that is predominately present on tumor cells, in tumor cells, or in tumor microenvironment, which can be used for treating one or more tumors. Tumor-associated antigen is distinguished from normal cellular proteins by distinct features in their levels of expression, localization, or major histocompatibility processing, which allows for their effective targeting in malignancies. Tumor-associated antigen can be broadly categorized into three groups: aberrantly expressed self-antigens, mutated self-antigens and tumor-specific antigens. Tumor-associated antigen as used herein includes an antigen that can be used as a target for treating one or more tumors, wherein its upregulation/activation or downregulation/inhibition is related to tumorigenesis or tumor progress, Tumor-associated antigen as used herein also denotes a peptide which has been isolated and identified from tumorous material and which underwent antigen processing in an antigen presenting cell and can thus be recognized by immune effector cells of the host. In particular, it refers to an antigen expressed exclusively on, associated with, or over-expressed in tumor tissue. A TAA peptide may comprise or consist of 5 to 20, 8 to 14, 8 to 12, for example 9 to 11 amino acids. In an aspect, TAA peptides that are capable of use with methods and embodiments described herein include, for example, those TAA peptides described in U.S. Publication 20160187351, U.S. Publication 20170165335, U.S. Publication 20170035807, U.S. Publication 20160280759, U.S. Publication 20160287687, U.S. Publication 20160346371, U.S. Publication 20160368965, U.S. Publication 20170022251, U. S. Publication 20170002055, U.S. Publication 20170029486, U.S. Publication 20170037089, U.S. Publication 2017013610, U.S. Publication 20170101473, U.S. Publication 20170096461, U.S. Publication 20170165337, U.S. Publication 20170189505, U.S. Publication 20170173132, U.S. Publication 20170296640, U.S. Publication 20170253633, U.S. Publication 20170260249, U.S. Publication 20180051080, and U.S. Publication No. 20180164315, the contents of each of these publications and sequence listings described therein are herein incorporated by reference in their entireties.

The term “viral antigen” refers to antigens derived from any disease-associated pathogenic virus. Exemplary disease-associated viral antigens include, but are not limited to, antigens derived from adenovirus, Coxsackievirus, Crimean-Congo hemorrhagic fever virus, cytomegalovirus (“CMV”), dengue virus, Ebola virus, Epstein-Barr virus (“EBV”), Guanarito virus, herpes simplex virus-type 1 (“HSV-1”), herpes simplex virus-type 2 (“HSV-2”) human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”) human immunodeficiency virus (“HIV”), influenza virus, Junin virus, Lassa virus, Machupo virus, Marburg virus, measles virus, human metapneumovirus, mumps virus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus.

The term “bacterial antigen” refers to antigens derived from any disease-associated pathogenic virus. Exemplary bacterial antigens include, but are not limited to, antigens derived from Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium peringens, Clostridium tetani. Corynebacterium diptheriae, Enterococcus faecalis, Enterococcus faecium, Escherichia coli, enterotoxigenic Escherichia coli, enteropathogenic Escherichia coli, Escherichia coli) 157:H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

The term “fungal antigen” refers to antigens derived from any disease-associated pathogenic fungus, Exemplary fungal antigens include, but are not limited to, antigens derived from Aspergillus clavatus, Aspergillus flavus, Aspergillus fumigatus. Aspergillus nidulans, Aspergillus niger, Aspergillus terreus, Blastomyces dermatitidis, Candida albicans, Candida dubliniensis, Candida glabrata, Candida parapsilosis, Candida rugosa, Candida tropicalis, Cryptococcus albidus, Cryptococcus Cryptococcus laurentii, Cryptococcus neoformans, Histoplasma capsulatum, Microsporum canis, Pneumocystis carinii, Pneumocystis jirovecii, Sporothrix schenckii, Stachybotrys chartarum, Tinea barbae, Tinea capitis, Tinea corporis, Tinea cruris, Tinea faciei, Tinea incognito, Tinea nigra, Tinea versicolor, Trichophyton rubrum and Trichophyton tonsurans. The term “parasite antigen” refers to antigens derived from any disease-associated pathogenic parasite. Exemplary parasite antigens include, but are not limited to, antigens derived from Anisakis spp. Babesia spp., Baylisascaris procyonis, Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

### Antibody

In certain embodiments, the targeting moiety is an antibody or antigen-binding fragment thereof. By antigen-binding fragment, it is meant any antibody fragment that retains its binding activity to the target on the cancer cell, such as an scFv or other functional fragment including an immunoglobulin devoid of light chains, VHH, VNAR, Fab, Fab′, F(ab′)₂, Fv, antibody fragment, diabody, scAB, single-domain heavy chain antibody, single-domain light chain antibody, Fd, CDR regions, or any portion or peptide sequence of the antibody that is capable of binding antigen or epitope. VHH and VNAR are alternatives to classical antibodies and even though they are produced in different species (camelids and sharks, respectively). Unless specifically noted as “full length antibody.” when the disclosure refers to antibody it inherently includes a reference to an antigen-binding fragment thereof.

Certain targets of antibody, antigen-binding fragment, or protein (with examples of cancer cell types in parentheses) may include: Her2/Neu (Epithelial malignancies); CD22 (B cells, autoimmune or malignant); EpCAM (CD326) (Epithelial malignancies); EGFR (epithelial malignancies); PSMA (Prostate Carcinoma); CD30 (B cell malignancies); CD20 (B cells, autoimmune, allergic or malignant); CD33 (Myeloid malignancies); membrane IgE (Allergic B cells); IgE Receptor (CD23) (Mast cells or B cells in allergic disease), CD80 (B cells, autoimmune, allergic or malignant); CD86 (B cells, autoimmune, allergic or malignant); CD2 (T cell or NK cell lymphomas); CA125 (multiple cancers including Ovarian carcinoma); Carbonic Anhydrase IX (multiple cancers including Renal Cell Carcinoma); CD70 (B cells, autoimmune, allergic or malignant); CD74 (B cells, autoimmune, allergic or malignant); CD56 (T cell or NK cell lymphomas); CD40 (B cells, autoimmune, allergic or malignant); CD19 (B cells, autoimmune, allergic or malignant); c-met/HGFR (Gastrointestinal tract and hepatic malignancies; TRAIL-R1 (multiple malignancies including ovarian and colorectal carcinoma); DRS (multiple malignancies including ovarian and colorectal carcinoma): PD-1 (B cells, autoimmune, allergic or malignant); PDL1 (Multiple malignancies including epithelial adenocarcinoma); IGF-1R (Most malignancies including epithelial adenocarcinoma); VEGF and VEGFR (Solid tumor and eye AMD), VEGF-R2 (The vasculature associated with the majority of malignancies including epithelial adenocarcinomas); Prostate stem cell antigen (PSCA) (Prostate Adenocarcinoma); MUC1 (Epithelial malignancies); CanAg (tumors such as carcinomas of the colon and pancreas); Mesothelin (many tumors including mesothelioma and ovarian and pancreatic adenocarcinoma); P-cadherin (Epithelial malignancies, including breast adenocarcinoma); Myostatin (GDF8) (many tumors including sarcoma and ovarian and pancreatic adenocarcinoma); Cripto (TDGF1) (Epithelial malignancies including colon, breast, lung, ovarian, and pancreatic cancers); ACVRL 1/ALK1 (multiple malignancies including leukemias and lymphomas); MUC5AC (Epithelial malignancies, including breast adenocarcinoma); CEACAM (Epithelial malignancies, including breast adenocarcinoma); CD137 (B cells or T cells, autoimmune, allergic or malignant); CXCR4 (B cells or T cells, autoimmune, allergic or malignant): Neuropilin 1 (Epithelial malignancies, including lung cancer); Glypicans (multiple cancers including liver, brain and breast cancers): HER3/EGFR (Epithelial malignancies); PDGFRa (Epithelial malignancies); EphA2 (multiple cancers including neuroblastoma, melanoma, breast cancer, and small cell lung carcinoma); CD38 (Myeloma); CD138 (Myeloma) α4-integrin (AML, myeloma, CLL, and most lymphomas), C5 complement (PNH, aHUS, gMG, and NMOSD), C3 complement (PNH), MASP-2 (IgA kidney disease), C5aR (solid tumor), CR1 (eye wAMD disease), C3b and CFH (PNH, aHUS, gMG, and NMOSD).

The FDA maintains listings of approved antibody drugs or therapeutic antibodies for treating cancer, See The Orange Book Online or Drugs@FDA on the FDA website. The FDA also maintains listings of clinical trials in progress for therapeutic antibodies in the clinicaltrials.gov database, which may be searched by disease names. These antibody drugs or therapeutic antibodies or their antigen-binding sections, which are specific for various disease-associated antigens or tumor-associated antigen, can be employed as or in the targeting moiety or effector moiety in the protein complex, related compositions, and related treatment methods disclosed herein.

Examples of these antibody drugs or therapeutic antibodies include, but not limited to, 3F8, 8H19, Abagovomab, Abciximab, Abituzumab, Abrilumab, Actoxumab, Abciximab, Abituzumab, Abrilumab, Actoxumab, Adalimumab, Adecatumumab, Aducanumab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab (=IMA-638) Apolizumab, Arcitumomab, Ascrinvacumab, Aselizumab, Atezolizumab, Atinumab, Atlizumab (=tocilizumab), Atorolimumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Blinatumomab, Blosozumab, Bococizumab, Brentuximab vedotin, Briakinumab, Brodalumab, Brolucizumab, Brontictuzumab, Canakinumab, Cantuzumab mertansine, Cantuzumab ravtansine, Caplacizumab, Capromab pendetide, Carlumab, Catumaxomab, cBR96-doxorubicin immunoconjugate, Cedelizumab, Cetuximab, Ch. 14.18, Citatuzumab bogatox, Cixutumumab, Clazakizumab, Clenoliximab, Clivatuzumab tetraxetan, Codrituzumab, Coltuximab ravtansine, Conatumumab, Concizumab, Crenezumab, CR6261, Dacetuzumab, Daclizumab, Dalotuzumab, Dapirolizumab pegol, Daratumuma, Dectrekumab, Demcizumab, Denintuzumab mafodotin, Denosumab, Derlotuximab biotin, Detumomab, Dinutuximab, Diridavumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Epratuzumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Gomiliximab, Guselkumab, ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Intetumumab, Inolimomab, Inotuzumab ozogamicin, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab. Lulizumab pegol, Lumiliximab, Lumretuzumab, Mapatumumab, Margetuximab, Maslimomab, Mavrilimumab, Matuzumab, Mepolizumab, Metelimumab, Milatuzumab, Minretumomab, Mirvetuximab soravtansine, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nemolizumab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Tetulomab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Rituximab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teplizumab, Teprotumumab, Tesidolumab, TGN1412, Ticilimumab (=tremelimumab), Tildrakizumab, Tigatuzumab, TNX-650, Tocilizumab (=atlizumab, Toralizumab, Tosatoxumab, Tositumomab, Tovetumab, Tralokinumab, Trastuzumab, Trastuzumab emtansine, TRBS07, Tregalizumab, Tremelimumab, Trevogrumab. Tucotuzumab celmoleukin, Tuvirumab, Ublituximab, Ulocuplumab, Urelumab, Urtoxazumab, Ustekinumab, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, and Zolimomab aritox. Additional examples include those described in U.S. Pat. Nos. 11,119,096, 11,091,562, and US 20210269547, the disclosures of which are incorporated by reference.

### Ligands

In some other embodiments, the targeting moiety can be or include a member of a binding pair while the other member of the binding pair is on a target of interest. Example of the binding pair include a ligand-receptor pair. In certain embodiments, a targeting moiety may be a binding partner for a protein know % n to be expressed on a cancer cell. Such expression levels may include overexpression.

Examples of the ligand may include IL-2, IL-4, IL-6, .alpha.-MSH, transferrin, folic acid, EGF, TGF, PDL IL-13, stem cell factor, insulin-like growth factor (IGF), CD40, or CD47. In some embodiments, the targeting moiety comprises a full-length sequence of IL-2, IL-4, IL-6, .alpha.-MSH, transferrin, folic acid, EGF, TGF, PD1, IL-L3, stem cell factor, insulin-like growth factor (ITF), or CD40. In some embodiments, the targeting moiety comprises a truncated form, analog, variant, or derivative of IL-2, IL-4, IL-6, α-MSH, transferrin, folic acid, LF, TGF, PD1, IL-13, stem cell factor, insulin-like growth factor (IGF), or CD40. In some embodiments, the targeting moiety binds a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

The binding partner need not comprise the full length or wildtype sequence for the binding partners. All that is required is that the binding partner bind to the target on the cancer cell and can thus include truncated forms, analogs, variants, and derivatives that are well known in the art.

### Others

In some embodiments, the targeting moiety capable of targeting a target (e.g., cancer) is not an antibody, but is another type of targeting moiety. A wide range of targeting moieties capable of targeting cancer are known, including DNA aptamers, RNA aptamers, albumins, lipocalins, fibronectins, ankyrins, CH1/2/3 scaffolds (including abdurins (IgG CH2 scaffolds)), fynomers, Obodies, DARPins, knotins, avimers, atrimers, anticalins, affilins, affibodies, bicyclic peptides, cys-knots, FN3 (adnectins, centyrins, protectins, TN3), and Kunitz domains. These and other non-antibody scaffold structures may be used for targeting to a cancer cell. Smaller non-antibody scaffolds are rapidly removed from the bloodstream and have a shorter half-life than monoclonal antibodies. They also show faster tissue penetration owing to fast extravasation from the capillary lumen through the vascular endothelium and basement membrane. See Vazquez-Lombardi et al., Drug Discovery Today 20(1:1271-1283 (2015). A number of non-antibody scaffolds targeting cancer are already under clinical development, with other candidates in the preclinical stage. See Vazquez-Lombardi et al, Drug Discovery Today 20(1):1271-1283 (2015), Table 1.

Additionally, in some embodiments, the binding partner may be an aptamer that is capable of binding to a protein know % n to be expressed on a cancer cell. Aptamers that bind cancer cells, such as cancer cells, are well known and methods for designing them are known.

### Effector Moiety

An effector moiety or effector domain or effector agent, used interchangeably herein, refers to an entity (e.g., an atom, a molecule, a compound, or a cell) which mediates a biological activity or response (e.g., immune response) or is useful for diagnostic or therapeutic application. An effector agent can be a diagnostic agent or a therapeutic agent.

A diagnostic effector moiety or domain or agent may be any entity that is useful in diagnosing a disease. Useful diagnostic agents include, but are not limited to, enzymes, DNAs, RNAs, peptides, substrates, chemiluminescence agents, radioisotopes, dyes, contrast agents, fluorescent compounds or molecules, enhancing agents (e.g., paramagnetic ions), or beads or other conjugates for collection. It is to be understood that a magnetic bead may be any suitable magnetic bead used for standard purification or separation. Accordingly, a magnetic bead may be ferromagnetic or paramagnetic or superparamagnetic, such as permanent magnets or materials attracted to magnetic materials.

A therapeutic effector moiety or domain or agent means any entity that may exert a therapeutic effect. Examples include immune checkpoint inhibitors, immune costimulatory/agonist agents (antibodies, ligands, or chemical agents), immune coinhibitory/antagonist agents (antibodies, protein, or chemical agents), cytokines, complement agents, cancer vaccines, anticancer agents, radioisotopes such as radioactive iodine-labeled compounds, toxins, cytostatic or cytolytic drugs, etc. Immune checkpoint inhibitors comprise, for example, antibodies or chemical agents against PD-1. PD-1, or CTLA-4. Immune costimulatory/agonist agents comprise, for example, antibodies, ligands, or chemical agents against 4-1BB, ICOS, GITR, CD70, CD27, OX40, or CD40. Immune coinhibitory/antagonist agents comprise, for example, antibodies, proteins, or chemical agents against VISTA, CCR4, B7-H3, TIM-3, LAG-3, KIR, IDO-1,2, TIGIT, A2aR, TGF-β, CD47, CD73, NKG2A, or NKG2B. Cytokines comprise, for example, IFN-α, IFN-β, IFN-γ, TFN-λ, IL-1β, IL2, IL6, IL-15, IL-17, or IL-12. Complement agents comprise, for example, antibodies, proteins, or chemical agents against C1r, C1s, C2, C3, C5, C5a, C5aR1, C6, MASPs, MSAP2, MASP3, FB, FD, or Properdin. Cancer vaccines comprise any cancer-specific antigens that can induce immune response of the body to attack the cancer. Anticancer agents comprise, for example, aminoglutethimide, azathioprine, bleomycin sulfate, busulfan, carmustine, chlorambucil, cisplatin, cyclophosphamide, cyclosporine, cytarabidine, dacarbazine, dactinomnycin, daunorubin, doxorubicin, taxol, etoposide, fluorouracil, interferon-.alpha., lomustine, mercaptopurine, methotrexate, mitotane, procarbazine HCl, thioguanine, vinblastine sulfate and vincristine sulfate, Other anticancer agents are described, for example, in Goodman and Gilman, “The Pharmacological Basis of Therapeutics”, 8th Edition, 1990, McGraw-Hill, Inc., in particular Chapter 52 (Antineoplastic Agents (Paul Calabresi and Bruce A. Chabner). Toxins may be proteins such a pokeweed antiviral protein, cholera toxin, pertussis toxin, ricin, gelonin, abrin, diphtheria exotoxin, Onconase (Ranpimase), or Pseudomonas exotoxin. Toxin residues may also be high energy-emitting radionuclides such as cobalt-60.

Other examples include cytotoxins or cytotoxic agents. A cytotoxin or cytotoxic agent includes any agent that is detrimental to and, in particular, kills cells. Useful classes of cytotoxic agents include, for example, oncolytic peptide, antitubulin agents, DNA minor groove binders (e.g., enediynes and lexitropsins), DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, nitrosoureas, platinols, pre-forming compounds, purine antimetabolites, puromycins, radiation sensitizers, steroids, taxanes (e.g., paclitaxel and docetaxel), topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, camptothecin, carboplatin, carmustine (BSNU, CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluorodeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, and anti-tubulin agents. Examples of anti-tubulin agents include, but are not limited to, dolastatins (e.g., auristatin E, AFP, MMAF, MMAE, AEB, AEVB), maytansinoids, taxanes (e.g., paclitaxel, docetaxel), T67 (Tularik), vinca alkyloids (e.g., vincristine, vinblastine, vindesine, and vinorelbine), baccatin derivatives, taxane analogs (e.g., epothilone A and B), nocodazole, colchicine and colcimid, estramustine, cryptophycins, cemadotin, combretastatins, discodermolide, and eleutherobin. Radioisotopes to generate cytotoxic radiopharmaceuticals include, e.g., iodine-131, yttrium-90 or indium-111.

Additional exemplary therapeutic agents that can be used as therapeutic effector moiety are described below in the section of Other Therapeutic Agents.

Techniques for conjugating such therapeutic effector moiety (drug) to antibodies, proteins, or peptides are well known. The generation of antibody/protein/peptide-drug conjugates can be accomplished by any technique known to the skilled artisan. A peptide and a drug may be directly bound to each other via their own linker groups or indirectly via a linker or other substance.

### Immune Cell Engaging Domain

In certain embodiments, the effector moiety can be or can include an immune cell engaging domain that can bind or recruit one or more immune cells. In some embodiments, the immune cell is a T cell, natural killer cell, macrophage, neutrophil, eosinophil, basophil, γδ T cell, NKT cell, or engineered immune cell.

### T Cell

For engaging a T cell, the effector moiety may bind to the CD3 antigen and/or T-cell receptor or any specific engaging marker on the surface of the T-cell. CD3 is present on all T cells and consists of subunits designated γ, δ, ε, ζ, and η. The cytoplasmic tail of CD3 is sufficient to transduce the signals necessary for T cell activation in the absence of the other components of the TCR receptor complex. Normally, activation of T cell cytotoxicity depends first on binding of the TCR with a major histocompatibility complex (MHC) protein, itself bound to a foreign antigen, located on a separate cell. In a normal situation, only when this initial TCR-MHC binding has taken place can the CD3 dependent signally cascade responsible for T cell clonal expansion and, ultimately, T cell cytotoxicity ensue. In some of the present embodiments, however, when the multispecific protein complex binds to CD3 and/or the TCR, activation of cytotoxic T cells in the absence of independent TCR-MHC can take place by virtue of the crosslinking of the CD3 and/or TCR molecules mimicking an immune synapse formation. This means that T cells may be cytotoxically activated in a clonally independent fashion, i.e. in a manner that is independent of the specific TCR clone carried by the T cell. This allows for activation of the entire T cell compartment rather than only specific T cells of a certain clonal identity.

In some embodiments, the T-cell engaging domain may comprise an scFv that is specific for an antigen expressed on the surface of a T cell, such as CD3 or TCR. If the antigen is CD3, one potential T-cell engaging domain may be derived from muromonab (muromonab-CD3 or OKT3), otelixizumab, teplizumab, visilizumab, foralumab, 20G6, or SP34. One skilled in the art would be aware of a wide range of anti-CD3 antibodies, some of which are approved therapies or have been clinically tested in human patients (see Kuhn and Weiner Immunotherapy 8(8):889-906 (2016)).

### Natural Killer Cell

In some embodiments, the immune cell engaging domain can be or can include a natural killer (NK) cell engaging domain that specifically binds to an antigen on the NK cell. The antigen on the surface of the NK cell may be NKG2D, CD16, NKp30, NKp44, NKp46 or DNAM.

In some embodiments, having the effector moiety binding to a surface protein on the natural killer cell and having the targeting moiety binding to a target cell (e.g., a pathogen, a disease cell, or a cancer cell) allows specific engagement of natural killer cells. Engagement of natural killer cells can lead to their activation and induce natural killer cell-mediated cytotoxicity and cytokine release.

The natural killer cell may specifically lyse the target cells bound by the protein complex. Killing of a target cell may be mediated by either the perforin/granzyme system or by FasL-Fas engagement. As well as this potential cytotoxic function, natural killer cells are also able to secrete pro-inflammatory cytokines including interferon gamma, and tumor necrosis factor alpha which can activate macrophages and dendritic cells in the immediate vicinity to enhance the anti-target (e.g., anti-cancer) immune response. The natural killer cell engaging domain may comprise an scFv, Fab, or antigen-binding fragment that is specific for an antigen expressed on the surface of a natural killer cell, such as NKG2D, CD16, NKp30, NKp44, NKp46 and DNAM.

### Macrophage

In some embodiments, the immune cell engaging domain can be or include a macrophage engaging domain. As used herein, a “macrophage” may refer to any cell of the mononuclear phagocytic system, such as grouped lineage-committed bone marrow precursors, circulating monocytes, resident macrophages, and dendritic cells (DC). Examples of resident macrophages can include Kupffer cells and microglia. The macrophage engaging domain binds specifically to an antigen on the surface of the macrophage to engage these cells. In some embodiments, the antigen on the surface of the macrophage may be CD89 (Fc alpha receptor 1), CD64 (Fc gamma receptor 1), CD32 (Fc gamma receptor 2A) or CD16a (Fc gamma receptor 3A).

Having the effector moiety binding to a surface protein on the macrophage cell and having the targeting moiety binding to a target cell (e.g., a pathogen, a disease cell, or a cancer cell) allows specific engagement of macrophages. Engagement of macrophages can lead the macrophage to phagocytose the target cell.

In some embodiments, inducing macrophage phagocytosis via binding to an antigen on the surface of the macrophages is independent of Fc receptor binding, which has been shown previously to be a method of target (e.g., tumor) cell killing by macrophages. Normally, cancer cells are bound by whole antibodies and the Fc portion of the antibody binds to the Fc receptor and induces phagocytosis. In some embodiments, engagement of toll-like receptors on the macrophage surface (see patent application US2150125397A1) leads to engagement of macrophages. The macrophage engaging domain may comprise an scFv, Fab, or antigen-binding fragment that is specific for an antigen expressed on the surface of a macrophage, such as CD89, CD64, CD32, CD16a, or toll-like receptors.

### Neutrophil

In some embodiments, the immune cell engaging domain can be or include a neutrophil engaging domain that specifically binds to an antigen on a neutrophil. Examples of the antigen on the surface of the neutrophil may be CD89 (FcαRI), FcγRI (CD64), FcγRIIA (CD32), FcγRIIIA (CD16a), CD11b (CR3, αMβ2), TLR2, TLR4, CLEC7A (Dectin1), formyl peptide receptor 1 (FPR1), formyl peptide receptor 2 (FPR2), or formyl peptide receptor 3 (FPR3).

In some embodiments, having the effector moiety binding to a surface protein on the neutrophil and having the targeting moiety binding to a target cell (e.g., a pathogen, a disease cell, or a cancer cell) allows specific engagement of neutrophils. Engagement of neutrophils can lead to phagocytosis and target cell uptake. That is, the neutrophil may engulf the target cells. The neutrophil engaging domain may comprise an scFv, Fab, or antigen-binding fragment specific for an antigen expressed on the surface of a neutrophil, such as any of those described above.

### Eosinophil

In some embodiments, the immune cell engaging domain can be or include an eosinophil engaging domain that specifically binds to an antigen on eosinophil. Examples of an antigen on the surface of the eosinophil include CD89 (Fc alpha receptor 1), FcεRI, FcγRI (CD64), FcγRIIA (CD32), FcγRIIIB (CD16b), or TLR4.

In sone embodiments, having the effector moiety binding to a surface protein on the eosinophil and having the targeting moiety binding to a target cell (e.g., a pathogen, a disease cell, or a cancer cell) allows specific engagement of eosinophils. Engagement of eosinophils can lead to degranulation and release of preformed cationic proteins, such as EPO, major basic protein 1 (MBP1), and eosinophil-associated ribonucleases (EARs), known as ECP and eosinophil-derived neurotoxin. In that case, the eosinophil may phagocytose the target cell or secrete neutrophil extracellular traps (NETs); finally, they may activate their respiratory burst cascade to kill phagocytosed cells.

The eosinophil engaging domain may comprise an scFv, Fab, or antigen-binding fragment specific for an antigen expressed on the surface of an eosinophil, such as any of those described above.

### Basophil

In some embodiments, the immune cell engaging domain can be or include a basophil engaging domain that specifically binds to an antigen on a basophil. Examples of an antigen on the surface of the basophil may be CD89 (Fc alpha receptor 1) or FcεRT.

In some embodiments, having the effector moiety binding to a surface protein on basophil and having the targeting moiety binding to a target cell (e.g. a pathogen, a disease cell, or a cancer cell) allows specific engagement of basophils. Engagement of basophils can lead to the release of basophil granule components such as histamine, proteoglycans, and proteolytic enzymes. They also secrete leukotrienes (LTD-4) and cytokines.

In some embodiments, the basophil engaging domain may comprise an scFv, Fab, or antigen-binding fragment that is specific for an antigen expressed on the surface of a basophil, such as any of those described above.

In some embodiments, the immune cell engaging domain can be or include a γδT-cell engaging domain. As used herein, a γδ T cell refers to a T cell having a TCR made up of one gamma chain (γ) and one delta chain (δ). The γδT-cell engaging domain specifically binds to an antigen on the surface of the γδ T cell to engage these cells. Examples of an antigen on the surface of the γδT cell include γδTCR, NKG2D, CD3 Complex (CD3ε, CD3γ, CD3δ, CD3ζ, and CD3η), 4-1BB, DNAM-1, or TLRs (e.g., TLR2, TLR6).

In some embodiments, having the effector moiety binding to a surface protein on γδT cell and having the targeting moiety binding to a target cell (e.g., a pathogen, a disease cell, or a cancer cell) allows specific engagement of γδT cells. Engagement of γδT cells can lead to cytolysis of the target cell and release of proinflammatory cytokines such as TNFα and IFNγ. In that case, γδT cells may kill the target cell.

In some embodiments, the γδT cell engaging domain may comprise an scFv, Fab, or antigen-binding fragment that is specific for an antigen expressed on the surface of a γδT cell, such as any of those described above.

### NKT Cell

In some embodiments, the immune cell engaging domain can be or include a NKT engaging domain. NKT cells refers to T cells that express the Vα24 and Vβ11 TCR receptors. The NKT engaging domain specifically binds to an antigen on the surface of the NKT to engage these cells. Examples of the antigen on the surface of the NKT include αβTCR, NKG2D, CD3 Complex (CD3ε, CD3γ, CD3δ, CD3ζ, and CD3η), 4-1BB, or IL-12R.

In some embodiments, having the effector moiety binding to a surface protein on the NKT and having the targeting moiety binding to a target cell (e.g., a pathogen, a disease cell, or a cancer cell) allows specific engagement of NKT. Engagement of NKTs can lead to cytolysis of the target cell. In that case, the NKT may cytolysis of the target cell and the release of proinflammatory cytokines.

In some embodiments, the NKT engaging domain may comprise an scFv, Fab, or antigen-binding fragment that is specific for an antigen expressed on the surface of a NKT, such as such as any of those described above.

In some embodiments, the immune cell engaging domain can be or include an engineered immune cell engaging domain. The engaging domain specifically binds to an antigen on the surface of the engineered immune cell to engage these cells. In some embodiments, the antigen on the surface of the engineered immune cell may be an engagement domain recited herein with specificity for T cells, NK cells, NKT cells, or γδT cells.

In some embodiments, the engineered immune cell is a chimeric antigen receptor (CAR) cell. The CAR may comprise an extracellular domain (for example, an scFv) capable of tightly binding to a tumor antigen, fused to a signaling domain partly derived from a receptor naturally expressed by an immune cell. Exemplary CARs are described in Facts about Chimeric Antigen Receptor (CAR) T-Cell Therapy, Leukemia and Lymphoma Society, December 2017.

CARs may comprise an scFV region specific for a target (such as a tumor antigen), an intracellular co-stimulatory domain, and linker and transmembrane region. For example, a CAR in a CAR T cell may comprise an extracellular domain targeting a tumor antigen fused to a signaling domain partly derived from the T cell receptor. A CAR may also comprise a co-stimulatory domain, such as CD28, 4-1 BB, or OX40. In some embodiments, binding of the CAR expressed by an immune cell to a tumor target antigen results in immune cell activation, proliferation, and target cell elimination. Thus, a range of CARs may be used that differ in their scFV region, intracellular co-stimulatory domains, and linker and transmembrane regions to generate engineered immune cells.

Exemplary engineered immune cells include CAR T cells, NK cells, NKT cells, and γδ T cells. In some embodiments, engineered immune cells can be derived from a patient's own immune cells or from a healthy donor. In some embodiments, the patient's tumor expresses a tumor antigen that binds to the scFV of the CAR.

Potential CAR targets studied so far include CD19, CD20, CD22, CD30, CD33, CD123, ROR1, Igk light chain, BCMA, LNGFR, and NKG2D. However, the CAR technology would be available for developing engineered immune cells to a range of tumor antigens.

Again having the effector moiety binding to a surface protein on the engineered immune cell and having the targeting moiety binding to a target cell (e.g., a pathogen, a disease cell, or a cancer cell) allows specific engagement of engineered immune cells. Engagement of engineered immune cells can lead to activation of the effector response of these cells such as cytolysis of their target, release of cytokines, and killing of the target cell. The engineered immune cell engaging domain may comprise an scFv that is specific for an antigen expressed on the surface of an engineered immune cell, based on the type of cell used for the engineering.

Various exemplary antibodies specific for an antigen expressed on the surface of a T cell, natural killer cell, macrophage, neutrophil, eosinophil, basophil, γδ T cell, NKT cell, or engineered immune cell are known in the art. Examples include those described in, e.g., US20210269547, the content of which is incorporated by reference.

The above-described targeting moiety and effector moiety can be conjugated together to form the protein complex using any methods known in the art. In preferred embodiments, the targeting moiety and effector moiety can be formed via the two DDD moieties and the AD moiety. As described herein, two DDD moieties form a dimer that binds to the AD moiety.

### DDD and AD

This DDD/AD approach takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264. Wong and Scott, Nat. Rev. Mol. Cell Biol. 2004; 5: 959). The DDD and AD peptides may be attached to any protein, peptide, or other molecule. Because the DDD sequences spontaneously dimerize and bind to the AD sequence, the technique allows the formation of complexes between any selected molecules that may be attached to DDD or AD sequences. See, e.g., U.S. Pat. Nos. 7,521,056; 7,527,787; 7,534,866; 7,550,143; 7,666,400; 7,901,680; 7,906,118; 7,981,398; and 8,003,111. The contents of these patents are incorporated herein by reference.

Although a standard DDD/AD complex comprises a trimer with two DDD-linked molecules attached to one AD-linked molecule, variations in complex structure allow the formation of dimers, trimers, tetramers, pentamers, hexamers and other multimers. In some embodiments, the complex described herein may comprise two or more antibodies, antibody fragments or fusion proteins which bind to the same antigenic determinant or to two or more different antigens. The complex may also comprise one or more other effectors, such as proteins, peptides, immunomodulators, cytokines, interleukins, interferons, binding proteins, peptide ligands, carrier proteins, toxins, ribonucleases such as onconase, inhibitory oligonucleotides such as siRNA, antigens or xenoantigens, polymers such as PEG, enzymes, therapeutic agents, hormones, cytotoxic agents, anti-angiogenic agents, pro-apoptotic agents or any other molecule or aggregate.

PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J, Biol. Chem. 1989; 264:8443). Isozymes of PKA are found with two types of R subunits (RI and RII), and each type has a and r isoforms (Scott. Pharmacol. Ther. 1991; 50:123). Thus, the four isoforms of PKA regulatory subunits are RIα, RIβ, RIIα, and RIIβ, each of which comprises a DDD moiety amino acid sequence. The RT a subunits have been isolated only as stable dimers and the dimerization domain has been shown to consist of the first 44 amino-terminal residues (Newlon et al., Nat. Struct. Biol. 1999; 6:222). As discussed below, similar portions of the amino acid sequences of other regulatory subunits are involved in dimerization and docking, each located near the N-terminal end of the regulatory subunit. Binding of cAMP to the R subunits leads to the release of active catalytic subunits for a broad spectrum of serine/threonine kinase activities, which are oriented toward selected substrates through the compartmentalization of PKA via its docking with AKAPs (Scott el al., J. Biol. Chem. 1990; 265; 21561).

Since the first AKAP, microtubule-associated protein-2, was characterized in 1984 (Lohmann et al., Proc. Natl. Acad. Sci USA 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004; 5:959). The AD of AKAPs for PIKA is an amphipathic helix of 14-18 residues (Carr et al., J. Biol. Chem. 1991; 266:14188), The amino acid sequences of the AD are varied among individual AKAPs, with the binding affinities reported for RIT dimers ranging from 2 to 90 nM (Alto et al., Proc. Natl. Acad. Sci. USA 2003; 100:4445). AK-APs will only bind to dimeric R subunits. For human RIIα, the AD binds to a hydrophobic surface formed by the 23 amino-terminal residues (Colledge and Scott, Trends Cell Biol. 1999; 6:216), Thus, the dimerization domain and AKAP binding domain of human RIIα are both located within the same N-terminal 44 amino acid sequence (Newlon et al. Nat. Struct. Biol. 1999; 6:222; Newlon et al., EMBO J. 2001; 20:1651), which is termed the DDD herein.

As described herein, the DDD of human PKA regulatory subunits and the AD of AKAP are used as a binding pair of linker modules for docking any two entities into a noncovalent complex, which could be further locked through the introduction of cysteine residues into both the DDD and AD at strategic positions to facilitate the formation of disulfide bonds.

Illustrated in FIGS. 1A-1D are schematics of four exemplary formats or models of the protein complex described herein. As exemplified in FIGS. 1A and 1B, the two light chains of one antibody (e.g. an anti-TAA antibody) are linked to two DDD sequences while another protein (e.g., a single chain anti-CD3 antibody) is linked to an AD sequence. Because the two DDD sequences would effect the spontaneous formation of a dimer, the two DDD-containing chains dimerize via the DDD sequences. The dimeric motif of DDD contained in the light chains creates a docking site for binding to the AD sequence contained in the other protein, thus facilitating a ready association of the two light chains and the other protein (anti-CD3 antibody in this particular example) to form a binary, trimeric complex having two antigen sites for one antigen (e.g., TAA) and one antigen site for another (e.g., CD3), a “2+1” format.

Similarly, as exemplified in FIGS. 1C and 1D, the two heavy chains of one antibody (e.g., an anti-TAA antibody) are linked to (FIG. 1D) or inserted with (FIG. 1C) two DDD sequences while another protein (e.g., a single chain anti-CD3 antibody) is linked to an AD sequence. The dimeric motif of DDD contained in the heavy chains creates a docking site for binding to the AD sequence contained in the other protein, thus facilitating a ready association of the two heavy chains and the other protein (anti-CD3 antibody in this particular example), thereby also forming a binary, trimeric complex of the “2+1” format.

This binding event can be stabilized with a subsequent reaction to covalently secure the two or three entities of the trimeric complex via disulfide bridges, which can occur very efficiently based on the principle of effective local concentration because the initial binding interactions should bring the reactive thiol groups placed onto both the DDD and AD into proximity (Chmura et al., Proc. Natl. Acad. Sci. USA 2001; 98:8480) to ligate site-specifically. For example, as shown in FIG. 1B, the two linkers connecting each pair of light chain and DDD sequences is stabilized with a disulfide bond. Such one or more disulfide bonds can also be strategically placed between any two or more chains in the complex. Using various combinations of linkers, adaptor modules and precursors, a wide variety of constructs of different stoichiometry may be produced and used (see, e.g., U.S. Pat. Nos. 7,550,143; 7,521,056; 7,534,866; 7,527,787 and 7,666,400).

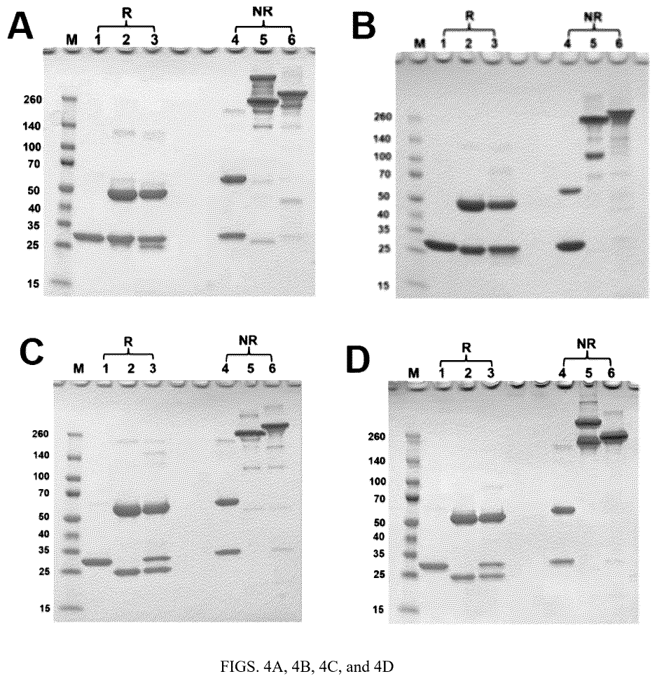

It was unexpected that all four formats in current disclosure can form functional binary, trimeric complexes despite multiple protein chains are conjugated together via multiple dimerization and trimerization domains. Although two free DDD-fused molecules (for example, two Fab-DDD molecules or two interferon-DDD molecules) can dimerize and assemble with an AD-fused molecule (for example, a scFv-AD2 molecule) to form a stable structure when covalently linked with disulfide bonds, it was unpredictable whether two DDD moieties could dimerize correctly (intramolecularly dimerize, intermolecularly crosslink or both) when they are fused to two light chains or two heavy chains of one immunoglobulin molecule. As disclosed herein, it was unexpected that, without the addition of AD-linked scFv, both IgG monomer and covalently linked IgG diner or multimer formed in Formats A and D as shown in FIGS. 4A and 4D. Moreover, without the addition of AD-linked scFv, some IgG monomer and most noncovalently linked dimeric IgG aggregate formed in Formats B and C, as detected by HPLC (data not shown). On the other hand, without the addition of DDD-linked IgG, the AD-linked scFv formed into both monomer and covalently linked dimer when it was produced alone (FIGS. 4A, 4B, 4C and 4D). It is indeed surprising when both AD-linked scFv and DDD-linked IgG were co-produced in one cell, they mostly assembled to form a stable monomer of IgG-scFv with minimal amount of dimer (FIGS. 4A, 4B, 4C and 4D) or low amount of aggregate (HPLC data not shown).

For Formats A and B, it was surprising to find that linking the other protein (e.g. a single chain anti-CD3 antibody) to the light chains via the DDD/AD trimer does not interfere with the assembly of the two heavy-light chain pairs and functions. For Format C, it was surprising to find that inserting a sequence (e.g., DDD sequence) in the hinge region or a flanking region does not prevent formation of the two heavy-light chain pairs and functions. It was also unexpected that DDDs inserted in the two heavy chains can still dimerize despite space constrains and dock with an AD sequence to form a timer. As to Format D, it was unexpected that formation of a DDD/AD trimer at the C-termini of the heavy chains does not interfere with the function of the Fc regions. Among the four Formats of complexes, Format C was a design with the highest unpredictability due to potential space constrains and unpredictable effects on the DDD dimerization, DDD-AD interaction, and the structure/function of IgG molecules when two DDD moieties were inserted into the flexible hinge region. However, it unexpectedly turned out that this format generated the best products in quality, purity, yield, and bioactivity.

In addition, by attaching the DDD and AD away from the functional groups of the first and second moieties, such site-specific ligations are expected to preserve the original activities of the two moieties. This approach is modular in nature and can be applied to link, site-specifically and covalently, a wide range of substances, including peptides, proteins, antibodies, antibody fragments, nucleic acid, chemicals, and other effector or targeting moieties with a wide range of activities. Utilizing the fusion protein method of constructing AD and DDD conjugated effector or targeting moieties described in the Examples below, virtually any protein or peptide may be incorporated. However, the technique is not limiting, and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques. For additional guidance, skilled artisans may consult Frederick M. Ausubel et at, Current Protocols in Molecular Biology, John Wiley & Sons, 2003; and Sambrook et al., Molecular Cloning, A Laboratory Manual,” Cold Spring Harbor Press, Cold Spring Harbor, NY, 2001).

In preferred embodiments, the AD and/or DDD moiety nay be attached to either the N-terminal end or C-terminal end or in the riddle of an effector or targeting protein or peptide. However, the skilled artisan will realize that the site of attachment of an AD or DDD moiety to an effector moiety may vary, depending on the chemical nature of the effector moiety and the part(s) of the effector or targeting moiety involved in its physiological activity. Site-specific attachment of a variety of effector or targeting moieties may be performed using techniques known in the art, such as the use of bivalent cross-linking reagents and/or other chemical conjugation techniques.

Various AD or DDD sequences may be used. Exemplary DDD and AD sequences include those in SEQ ID NOs: 14 and 16, those described in U.S. Pat. No. 9,315,567, and their functional variants.

| | | SEQ ID |
|---|---|---|
| Name | Sequence | NQ |
| DDD1 | SHIQIPPGLTELLQGYTVEVLRQ | 56 |
| | QPPDLVEFAVEYFTRLREARA | |
| DDD2 | CGHIQIPPGLTELLQGYTVEVLR | 57 |
| | QQPPDLVEFAVEYFTRLREARA | |
| DDD3 | SLRECELYVQKHNIQALLKDSIV | 58 |
| | QLCTARPERPMAFLREYFERLEK | |
| | EEAK | |
| DDD4 | SIEIPAGLTELLQGFTVEVLRHQ | 59 |
| | PADLLEFALQHFTRLQQENER | |
| DDD5 | SLKGCELYVQLHGIQQVLKDCIV | 60 |
| | HLCISKPERPMKFLREHFEKLEK | |
| | EENRQILA | |
| AD1 | QIEYLAKQIVDNAIQQA | 61 |
| AD2 | CGQIEYLAKQIVDNAIQQAGC | 14 |
| AD3 | CGFEELAWKIAKMIWSDVEQQGC | 62 |

The structure-function relationships of the AD and DDD domains have been the subject of investigation. See, e.g., Burns-Hamuro et al., 2005, Protein Sci 14:2982-92; Carr et al., 2001, J Biol Chem 276:17332-38; Alto el al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24.397-408. The contents of these publications are incorporated herein by reference. Kinderman et al. (2006, Mol Cell 24:397-408) examined the crystal structure of the AD-DDD binding interaction and concluded that the human DDD sequence contained a number of conserved amino acid residues that were important in either dimer formation or AKAP binding, underlined in the sequence below. (See also FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

(SEQ ID NO:_56)
SHIQIPPGLTELLQGYTYEVLRQQPPDLVEFAVEYFTRLREARA

As discussed in more detail below, conservative amino acid substitutions have been characterized for each of the twenty common L-amino acids. Thus, based on the data of Kinderman (2006) and conservative amino acid substitutions, potential alternative DDD sequences can be created based on the above sequence. The skilled artisan will realize that many other alternative species within the genus of DDD moieties can be constructed by standard techniques, for example using a commercial peptide synthesizer or well-known site-directed mutagenesis techniques. Listed below are some exemplary DDD variants.

(SEQ ID NO: 63)
THIQIPPGLTELLQGYTVEVLRQQPPDLVEFAVEYFTRLREARA
(SEQ ID NO: 64)
SKIQIPPGLTELLQGYTVEVLRQQPPDIVEFAVEYFTRLREARA
(SEQ ID NO: 65)
SRIQIPPGLTELLQGYTVEVLRQQPPDLVEFAVEYFTRLREARA
(SEQ ID NO: 66)
SHINIPPGLTELLQGYTVEVLRQQPPDLVEFAVEYFTRLREARA
(SEQ ID NO: 67)
SHIQIPPALTELLQGYTVEVLRQQPPDLVEFAVEYFTRIREARA
(SEQ ID NO: 68)
SHIQIPPGLSELLQGYTVEVLRQQPPDLVEFAVEYFTRLREARA
(SEQ ID NO: 69)
SHIQIPPGLTDLLQGYTVEVLRQQPPDLVEFAVEYFTRLREARA
(SEQ ID NO: 70)
SHIQIPPGLTELLNGYTVEVLRQQPPDLVEFAVEYFTRIREARA
(SEQ ID NO: 71)
SHIQIPPGLTELLQAYTVEVLRQQPPDLVEFAVEYFTRLREARA
(SEQ ID NO: 72)
SHIQIPPGLTELLQGYSVEVLRQQPPDLVEFAVEYFIRLREARA
(SEQ ID NO: 73)
SHIQIPPGLTELLQGYTVDVLRQQPPDLVEFAVEYFTRIREARA
(SEQ ID NO: 74)
SHIQIPPGLTELLQGYTVEVLKQQPPDLVEFAVEYFTRLREARA
(SEQ ID NO: 75)
SHIQIPPGLTELLQGYTVEVLRNQPPDLVEFAVEYFTRLREARA
(SEQ ID NO: 76)
SHIQIPPGLTELLQGYTVEVLRQNPPDLVEFAVEYFTRIREARA
(SEQ ID NO: 77)
SHIQIPPGLTELLQGYTVEVLRQQPPELVEFAVEYFTRLREARA
(SEQ ID NO: 78)
SHIQIPPGLTELLQGYTVEVLRQQPPDLVDFAVEYFTRLREARA
(SEQ ID NO: 79)
SHIQIPPGLTELLQGYTVEVLRQQPPDLVEFLVEYFTRIREARA
(SEQ ID NO: 80)
SHIQIPPGLTELLQGYTVEVLRQQPPDEVEFIVEYFTRLREARA
(SEQ ID NO: 81)
SHIQIPPGLTELLQGYTVEVLRQQPPDLVEFVVEYFTRLREARA
(SEQ ID NO: 82)
SHIQEPPGLTELLQGYTVEVLRQQPPDLVEFAVDYFTRIREARA

The N-terminal dimeric structure of RIIα (1-44) can be subdivided into two functional regions: the first 23 residues, which form the AD binding surface (docking domain), and residues 24-44, which encompass the bulk of the dimer contacts (dimerization domain) (Newlon et al., Nat. Struct. Biol. 1999; 6:222). If this N-terminal motif of RIIα is inserted into two self-dimerized heavy chains of immunoglobulin, it is possible that, without the dimerization domain, the docking domain (DD, at least amino acids 1-23: SHTQIPPGLTELLQGYTVEVLRQ, SEQ ID NO. 120) alone may still bind with an AD-fused moiety. Accordingly, in some embodiments, only the docking motif or its functional variant is used to form the complex or fusion protein described herein The effect of the amino acid substitutions on AD moiety binding may also be readily determined by standard binding assays, for example as disclosed in Alto et al. (2003, Proc Natl Acad Sci USA 100:4445-50). Specifically, Alto et al. performed a bioinformatic analysis of the AD sequence of various AKAP proteins to design a RII selective AD sequence called AKAP-IS, with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in the sequence below.

(SEQ ID NO: 61)
QIEYLAKQIVDNAIQQA

The skilled artisan will realize that in designing sequence variants of the AD sequence, one would desirably avoid changing any of the underlined residues, while conservative amino acid substitutions might be made for residues that are less critical for DDD binding. Listed below are some exemplary AD variants.

(SEQ ID NO: 83)
NIEYLAKQIVDNAIQQA
(SEQ ID NO: 84)
QLEYLAKQIVDNAIQQA
(SEQ ID NO: 85)
QVEYLAKQIVDNAIQQA
(SEQ ID NO: 86)
QIDYLAKQIVDNAIQQA
(SEQ ID NO: 87)
QIEFLAKQIVDNAIQQA
(SEQ ID NO: 88)
QIETLAKQIVDNAIQQA
(SEQ ID NO: 89)
QIESLAKQIVDNAIQQA
(SEQ ID NO: 90)
QIEYIAKQIVDNAIQQA
(SEQ ID NO: 91)
QIEYVAKQIVDNAIQQA
(SEQ ID NO: 92)
QIEYLARQIVDNAIQQA
(SEQ ID NO: 93)
QIEYLAKNIVDNAIQQA
(SEQ ID NO: 94)
QIEYLAKQIVENAIQQA
(SEQ ID NO: 95)
QIEYLAKQIVDQAIQQA
(SEQ ID NO: 96)
QIEYLAKQIVDNAINQA
(SEQ ID NO: 97)
QIEYLAKQIVDNAIQNA
(SEQ ID NO: 98)
QIEYLAKQIVDNAIQQL
(SEQ ID NO: 99)
QIEYLAKQIVDNAIQQI
(SEQ ID NO: 100)
QIEYLAKQIVDNAIQQV

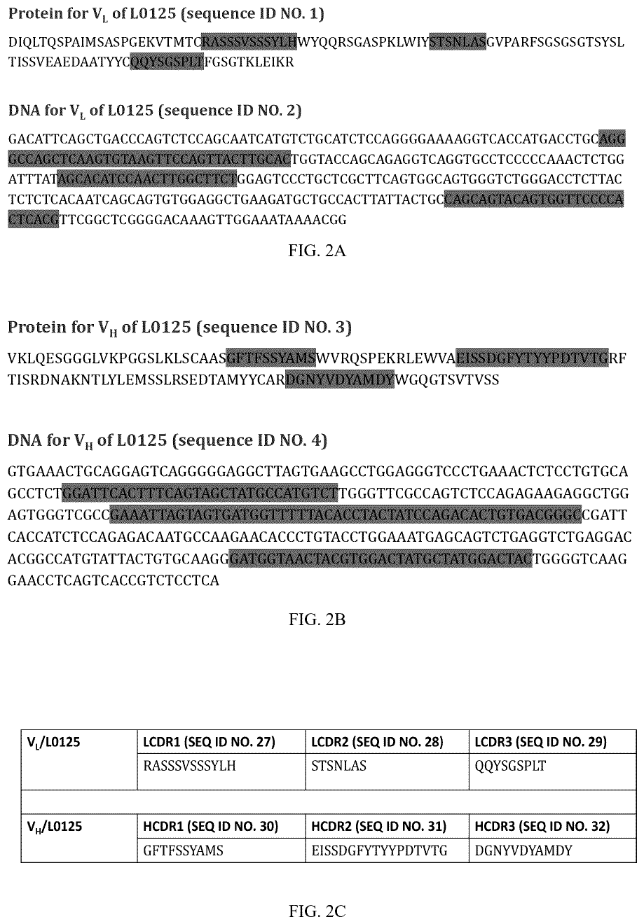

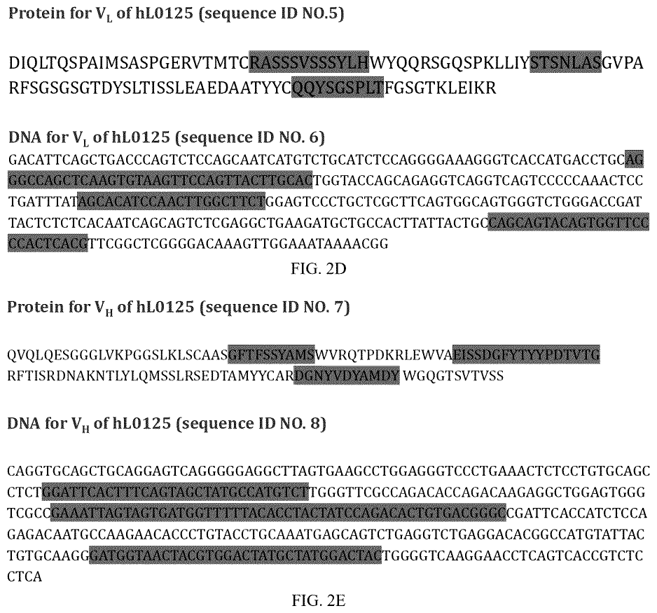

The skilled artisan will realize many other variant species within the genus of possible AD moiety sequences could be made, tested and used by the skilled artisan, based on the data of Alto et al. (2003). It is noted that FIG. 2 of Alto (2003) shows an even large number of potential amino acid substitutions that may be made, while retaining binding activity to DDD moieties, based on actual binding experiments. It is also noted that Gold el al. (2006, Mal Cell 24:383-95) utilized crystallography and peptide screening to develop a SuperAKAP-IS sequence (SEQ ID NO: 101), exhibiting a five order of magnitude higher selectivity for the RII isoform of PKA compared with the RI isoform. In this sequence, the N-terminal Q residue is numbered as residue number 4 and the C-terminal A residue is residue number 20. Residues where substitutions could be made to affect the affinity for RIIa were residues 8, 11, 15, 16, 18, 19 and 20 (Gold et al., 2006). It is contemplated that in certain alternative embodiments, the SuperAKAP-TS sequence may be substituted for the AKAP-TS AD moiety sequence to construct any of four Formats of complexes. Other alternative sequences that might be substituted for the AKAP-IS AD sequence are shown in SEQ ID NO: 102-105. Additional DDD-binding sequences were discovered from a variety of AKAP proteins or developed as peptide competitors of AKAP binding to PKA, as shown in SEQ ID NOs: 106-109, 121-130 (Gold et al., 2006). It is anticipated that, as with the AD2 sequence shown in SEQ ID NO: 14, the AD moiety may also include the additional N-terminal residues cysteine and glycine and C-terminal residues glycine and cysteine.

(SEQ ID NO: 101)
QIEYVAKQIVDYAIHQA
(SEQ ID NO: 102)
QIEYLAKQIVDYAIHQA
(SEQ ID NO: 103)
QIEYKAKQIVDHAIHQA
(SEQ ID NO: 104)
QIEYHAKQIVDHAIHQA
(SEQ ID NO: 105)
QIEYVAKQIVDHAIHQA
(SEQ ID NO: 106)
LIEEAASRIVDAVIEQV
(SEQ ID NO: 107)
PLEYQAGLIVQNAIQQA
(SEQ ID NO: 108)
KGADLIEEAASRIVDAVIEQVKAAG
(SEQ ID NO: 109)
PEDAELVRLSKRIVENAVLKAVQQY

### RII-Specific AKAPs

AKAP-KL:
(SEQ ID NO: 121)
PLEYQAGLIVQNAIQQAI
AKAP79:
(SEQ ID NO: 122)
LLIETASSLVKNAIQLSI
AKAP-Lbc:
(SEQ ID NO: 123)
LIEEAASRIVDAVIEQVK

### RI-Specific AKAPs

AKAPce:
(SEQ ID NO: 124)
ALYQFADRESELVISEAL
RIAD:
(SEQ ID NO: 125)
LEQYANQLADQIIKEAT
PV38:
(SEQ ID NO: 126)
FEELAWKIAKMIWSDVE

### Dual-Specificity AKAPs

AKAP7:
(SEQ ID NO: 127)
ELVRLSKRIVENAVLKAV
MAP2D:
(SEQ ID NO: 128)
TAEEVSARIVQVVTAEAV
DAKAP1:
(SEQ ID NO: 129)
QIKQAAFQLISQVILEAT
DAKAP2:
(SEQ ID NO: 130)
LAWKIAKMIVSDVMQQ

Hundsrucker et al. (2006, Biochem J 396: 297-306) developed additional peptide competitors for AKAP binding to PKA and demonstrated that peptides (25 amino acid residues) derived from the RIT-binding domain of AKAP7δ bind RIIα subunits with higher affinity (Kd=0.4±0.3 nM) than either full length or N-terminally truncated AKAP7δ, or peptides derived from other RII binding domains. Alanine scanning (25 amino acid peptides, SPOT technology, combined with RII overlay assays) of the RII binding domain revealed that hydrophobic amino acid residues form the backbone of the interaction and that hydrogen bond- and salt-bridge-forming amino acid residues increase the affinity of the interaction. The sequences of some AKAP antagonistic peptide are listed below (SEQ ID NOs: 131-147). The skilled artisan will realize many other variant species within the genus of possible AD moiety sequences could be made, tested and used by the skilled artisan, based on the data of Hundsrucker et al. (2006). It is anticipated that, as with the AD2 sequence shown in SEQ ID NO: 14, the AD moiety may also include the additional N-terminal residues cysteine and glycine and C-terminal residues glycine and cysteine.

AKAPIS-P:
(SEQ ID NO: 131)
QIEYLAKQIPDNAIQQA
Ht31:
(SEQ ID NO: 132)
KGADLIEEAASRIVDAVIEQVKAAG
Ht31-P:
(SEQ ID NO: 133)
KGADLIEEAASRIPDAPIEQVKAAG
AKAP7δ-wt-pep:
(SEQ ID 134)
PEDAELVRLSKRIVENAVLKAVQQY
AKAP7δ-1304T-pep:
(SEQ ID NO: 135)
PEDAELVRTSKRIVENAVLKAVQQY
AKAP7δ-P-pep:
(SEQ ID NO: 136)
PEDAELVRLSKRLPENAVLKAVQQY
AKAP7δ-D-pep:
(SEQ ID NO: 137)
PDDAELVRLSKRIVENAVLKAVQQY
AKAP1-pep:
(SEQ ID NO: 138)
PEDAELVRLSKRIVENAVEKAVQQY
(SEQ ID NO: 139)
EEGLDRNEEIKRAAFQIISQVISEA
AKAP2-pep:
(SEQ ID NO: 140)
LVDDPLEYQAGLLVQNAIQQAIAEQ
AKAP5-pep:
(SEQ ID NO: 141)
QYETLLIETASSLVKNAIQLSIEQL
AKAP9-pep:
(SEQ ID NO: 142)
LEKQYQEQLEEEVAKVIVSMSIAFA
AKAP10-pep:
(SEQ ID NO: 143)
NTDEAQEELAWKIAKMIVSDIMQQA
AKAP11-pep:
(SEQ ID NO: 144)
VNLDKKAVLAEKIVAEAIEKAEREL
AKAP12-pep:
(SEQ ID NO: 145)
NGILELETKSSKLVQNIIQTAVDQF
AKAP14-pep:
(SEQ ID NO: 146)
TQDKNYEDELTQVALALVEDVINYA
Rab32-pep:
(SEQ ID NO: 147)
ETSAKDNINIEEAARFLVEKILVNH

### Linkers

The above-mentioned AD or DDD domain as well as others as disclosed herein can be joined to another protein by means of linkers, such as, but not limited to chemical modification, peptide linkers, chemical linkers, covalent or non-covalent bonds, or protein fusion or by any means known to one skilled in the art. The joining can be permanent or reversible. See for example U.S. Pat. Nos. 4,625,014, 5,057,301 and 5,514,363, US Application Nos. 20150182596 and 20100063258, and WO2012142515, the contents of which are incorporated herein in their entirety by reference.

In some embodiments, several linkers can be included in order to take advantage of desired properties of each linker and each protein domain in the conjugate. For example, flexible linkers and linkers that increase the solubility of the complex are contemplated for use alone or with other linkers. Peptide linkers can be linked by expressing DNA encoding the linker to one or more protein domains in the conjugate. Linkers can be acid cleavable, photocleavable and heat sensitive linkers. Methods for conjugation are well known by persons skilled in the art and are encompassed for use in the present invention.

Suitable examples of the linkers include peptides, polymers, nucleotides, nucleic acids, polysaccharides, and lipid organic species (such as polyethylene glycol). In some embodiments, the linker is a peptide linker. Peptide linkers may be from about 2-100, 10-50, or 15-30 amino acids long. In some embodiments, peptide linkers may be at least 10, at least 15, or at least 20 amino acids long and no more than 80, no more than 90, or no more than 100 amino acids long. In some embodiments, the linker is a peptide linker that has a single or repeating GGGGS, GGGS, GS, GSGGS, GGSG, GGSGG, GSGSG, GSGGG, GGGSG, and/or GSSSG sequence(s) (SEQ ID NOs: 110-119).

In some embodiments, the AD or DDD domain and another protein domain can be joined by a peptide linker. Peptide linkers can be linked by expressing nucleic acid encoding in frame the two domains and the linker. Optionally the linker peptide can be joined at either or both of the amino terminus and carboxy terminus of the domains. In some examples, a linker is an immunoglobulin hinge region linker as disclosed in U.S. Pat. Nos. 6,165,476, 5,856,456, US Application Nos. 20150182596 and 2010/0063258 and International Application WO2012/142515, each of which are incorporated herein in their entirety by reference.

### New Anti-TROP2 Antibodies

The disclosure provides novel anti-TROP2 antibodies or antigen-binding fragments thereof. Trop2 is also known as tumor-associated calcium signal transducer 2, trophoblast antigen 2, cell surface glycoprotein Trop-2/Trop2, gastrointestinal tumor-associated antigen GA7331, pancreatic carcinoma marker protein GA733-1/GA733, membrane component chromosome 1 surface marker 1 MIS1, epithelial glycoprotein-1 (EGP-1), CAA1, Gelatinous Drop-Like Corneal Dystrophy GDLD, and TTD2. It is coded by the gene Tacstd2 and the TACSTD2 gene in human.

This gene encodes a carcinoma-associated antigen, which is a member of a family including at least two type I membrane proteins. It transduces an intracellular calcium signal and acts as a cell surface receptor. Mutations of this gene result in conditions including gelatinous drop-like corneal dystrophy, an autosomal recessive disorder characterized by severe corneal amyloidosis leading to blindness. The transmembrane glycoprotein Trop2 is highly expressed in many cancers, but not all, and has differential expression in certain normal tissues. It is about 35 kDa. Trop2 spans the cellular membrane: it has an extracellular, a transmembrane, and an intracellular domain, along with a cytoplasmic tail essential for signaling (Shvartsur et al., Genes Cancer. 2015 March; 6(3-4): 84-105.). Trop-2 is upregulated in many cancer types independent of baseline levels of Trop-2 expression. Trop-2 is an ideal candidate for targeted therapeutics due to it being a transmembrane protein with an extracellular domain overexpressed on a wide variety of tumors as well as its upregulated expression relative to normal cells. Zaman et al. Onco Targets Ther. 2019; 12: 1781-1790.

The anti-TROP2 antibodies or antigen-binding fragments thereof described herein can be used to treat multiple cancers and tumor types. Examples include, but not limited to, breast cancer (e.g., triple-negative breast cancer), urothelial cancer (e.g., platinum-resistant urothelial cancer), and lung cancer (e.g., small-cell lung cancer).

Shown in FIGS. 2A-2E are CDR sequences, light chain variable region sequences, and heavy chain variable region sequences of an exemplary anti-TROP2 antibody L0125 and its humanized version hL0125. The antibodies may be used to treat and protect a subject prophylactically and therapeutically against a tumor or cancer.

In some embodiments, the antibody or antigen-binding fragment thereof comprising: three heavy chain complementarity determining regions (HCDRs) (HCDR1, HCDR2, and HCDR3) of a heavy chain variable region having an amino acid sequence of one selected from those in FIG. 2; and three light chain CDRs (LCDR1, LCDR2, and LCDR3) of a light chain variable region having the amino acid sequence of one selected from those in FIG. 2.

In some embodiments, the antibody or antigen-binding fragment thereof comprising: a light chain variable region having an amino acid sequence with at least 75% (e.g., 75%, 80%, 35%, 90%, 95%, 96%, 97%, 98%, 99%, 100%) identity to the amino acid sequence of SEQ ID NO: 1 or 5 or having the amino acid sequence of SEQ ID NO: 1 or 5; and a heavy chain variable region having an amino acid sequence with at least 75% (e.g. 75%, 80%, 85%, 90%, 95%, 96%, 97%, 98%, 99%, 100%) identity to the amino acid sequence of SEQ ID NO: 3 or 7 or having the amino acid sequence of SEQ ID NO: 3 or 7.

In some embodiments, the antibody or the antigen-binding fragment thereof further comprises a variant Fc region (e.g., a variant Fc region containing E233P/L234V, L235A, G236del, and S267K substitutions according to the EU numbering). In some embodiments, the antibody is a monoclonal antibody. In some embodiments, the antibody is a chimeric antibody, a humanized antibody, or a humanized monoclonal antibody. In some embodiments, the antibody is a single-chain antibody, a Fab or a Fab2 fragment.

In some embodiments, the antibody or antigen-binding fragment thereof can be detectably labeled or conjugated to a toxin, a therapeutic agent, a polymer (e.g., poly ethylene glycol (PEG)), a receptor, an enzyme or a receptor ligand. For example, an antibody of the present disclosure may be coupled to a toxin (e.g., a tetanus toxin). Such antibodies may be used to treat animals, including humans, that have cancer.

In another example, an antibody of the present disclosure may be coupled to a detectable tag. Such antibodies may be used within diagnostic assays to determine if an animal, such as a human, has a cancer associated with TROP2 expression. Examples of detectable tags include fluorescent proteins (I.e., green fluorescent protein, red fluorescent protein, yellow fluorescent protein), fluorescent markers (i.e., fluorescein isothiocyanate, rhodamine, texas red), radiolabels (i.e., 31H, 32P, 125I), enzymes (i.e., 0-galactosidase, horseradish peroxidase, f-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

### Fragment

In some embodiments, an antibody provided herein is an antibody fragment. Antibody fragments include, but are not limited to, Fab, Fab′, Fab′-SH, F(ab′)2, Fv, and single-chain Fv (scFv) fragments, and other fragments described below, e.g., diabodies, triabodies tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments, an antibody provided herein is a chimeric antibody. Certain chimeric antibodies are described, e.g., in U.S. Pat. No. 4,816,567; and Morrison e al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci, 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol, Immunol, 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA. 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al, J. Biol. Chem. 271:22611-22618 (1996)).

### Human Antibodies

In some embodiments, an antibody provided herein is a human antibody. Human antibodies can be produced using various techniques known in the art or using techniques described herein. Human antibodies are described generally in van Dijk and van de Winkel, Curr. Opin. Pharmacol. 5: 368-74 (2001) and Lonberg, Curr. Opin. Immunol, 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987), and Boemer et al., J. Immunol., 147: 86 (1991).) Human antibodies generated via human B-cell hybridoma technology are also described in Li et al. Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianvixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone with variable domain sequences selected from human-derived phage display libraries. Such variable domain sequences may then be combined with a desired human constant domain. Techniques for selecting human antibodies from antibody libraries are described below.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev, Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

### Variants

In some embodiments, amino acid sequence variants of the antibodies and any components of the above-described protein complex are contemplated. The components can be any of the immunoglobulin chains, targeting moiety/agent, effector moiety/agent, DDD moiety, and AD moiety.

For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### Substitution, Insertion, and Deletion Variants

In some embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g. retained/improved antigen binding, decreased immunogenicity, or improved antibody-dependent cell-mediated cytotoxicity (ADCC) and complement-dependent cytotoxicity (CDC).

Accordingly, an antibody of the disclosure can comprise one or more conservative modifications of the CDRs, heavy chain variable region, or light variable regions described herein. A conservative modification or functional equivalent of a peptide, polypeptide, or protein disclosed in this disclosure refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this disclosure). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this disclosure are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

In some embodiments, the disclosed methods and compositions may involve production and use of proteins or peptides with one or more substituted amino acid residues. For example, the DDD and/or AD sequence variants have been discussed above.

The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyle & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within .+−0.2 is preferred, within .+−0.1 are more preferred, and within .+−0.0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasmnan, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, vat; Arg (R) gln, asn, lys; Asn (N) his, asp, lys, arg, gin; Asp (D) asn, glu; Cys (C) ala, ser; Gln (q) glu, asn; Gln (E) gln, asp; Gly (G) ala; H is (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y′) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

### Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta(1,4)-N-acetylglucosaminyltransferase III (GnTIII)) such that antibodies expressed in the engineered cell lines exhibit increased bisecting GlcNac structures which result in increased ADCC activity of the antibodies (see also Umana et al. (1999) Nat. Biotech. 17: 176-180).

### Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1: Glm, Glm1(a), Glm2(x), Glm3(f), Gim17(z); for IgG2: G2m, G2m23(n); for IgG3: C3n, G3m21(g1), G3m28(g5), G3 m1 1(b0), G3m5(b1), 03m13(b3), G3m14(b4), n3m10(b5), G3m15(s), G3m16(t), G3m6(c3), G3n24(c5), G3m26(u), G3m27(v); and for K: Km, Km1, Km2, Km3 (see, e.g., Jefferies et al. (2009) mAbs 1: 1). In some embodiments, the antibodies variable regions described herein are linked to an Fc that binds to one or more activating Fc receptors (FcγI, FcγIIa or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g. one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, Ig4, and other classes such as IgA, IgD, IgE and IgM. The constant region of an immunoglobulin is defined as a naturally-occurring or synthetically-produced polypeptide homologous to the immunoglobulin C-terminal region and can include a CH1 domain, a hinge, a CH2 domain, a CH3 domain, or a CH4 domain, separately or in combination. The antibody can have an Fc region from that of IgG (e.g., IgG1, IgG2, IgG3, and IgG4) or other classes such as IgA. IgD, IgE, and IgM.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2 IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains. The serum half-life of an antibody is influenced by the ability of that antibody to bind to an FcRn.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for C1q and/or (d) has increased or decreased affinity for an Fc receptor relative to the parent Fe. Such Fc region variants will generally comprise at least one amino acid modification in the Fc region. Combining amino acid modifications is thought to be particularly desirable. For example, the variant Fc region may include two, three, four, five, etc., substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together noncovalently. In other embodiments, the Fc region may be modified to make it more compatible with a selected host cell. For example, one may remove the PA sequence near the N-terminus of a typical native Fc region, which may be recognized by a digestive enzyme in E. coli, such as proline iminopeptidase. In other embodiments, one or more glycosylation sites within the Fc domain nay be removed. Residues that are typically glycosylated (e.g. asparagine) may confer cytolytic response. Such residues may be deleted or substituted with unglycosylated residues (e.g., alanine). In other embodiments, sites involved in interaction with complement, such as the C1q binding site, may be removed from the Fc region. For example, one may delete or substitute the EKK sequence of human IgG1. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1, Specific examples of variant Fe domains are disclosed, for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer el ai. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward e al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered C1q binding and/or reduced or abolished CDC. This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338,340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 3321), and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 362A, 247I, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in abat (WO00/42072).

Other Fc modifications that can be made to Fes are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary-substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fe variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 2341, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E. 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art, including but not limited to, equilibrium methods (e.g., ELISA, or radioimmunoassay), or kinetics (e.g. BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods, including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341H, 434F, 434Y, and 434M. Other variants that increase Fc binding to FcRn include: 250E. 250Q, 428L, 428F, 250Q/428L (Hinton et al., 2004, J. Biol. Chem. 279(8): 6213-62, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgG1/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 422I, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 H.

Moreover, the binding sites on human IgG1 for FcγRI, FcγRII, FcγRIII, and FcRn have been mapped and variants with improved binding have been described (see Shields, R. L. et al. (2001) J. Biol, Chem. 276:6591-6604). Specific mutations at positions 256, 290, 298, 333, 334, and 339 were shown to improve binding to FcγRIII. Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgG1 variants With strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007: Nordstrom et al., 2011). Other Fc mutants that may be used include: S298A/E333A/L334A, S239D/I332E, S239D/I332E/A330L, L235V/F243L/R292P′Y300L/P396L, and M428L/N434S.

In some embodiments, an Fc is chosen that has reduced binding to FcγRs. An exemplary Fc, e.g., IgG1 Fc, with reduced FcγR binding, comprises the following three amino acid substitutions: L234A, L235E, and G237A.

In some embodiments, an Fc is chosen that has reduced complement fixation. An exemplary Fc, e.g., IgG1 Fe, with reduced complement fixation, has the following two amino acid substitutions: A330S and P331S.

In some embodiments, an Fc is chosen that has essentially no effector function, i.e., it has reduced binding to FcγRs and reduced complement fixation. An exemplary Fc, e.g., IgG1 Fe, that is effectorless, comprises the following five mutations: L234A, L235E, G237A, A330S, and P331S.

When using an IgG4 constant domain, it is usually preferable to include the substitution S228P, which mimics the hinge sequence in IgG1 and thereby stabilizes IgG4 molecules.

### Multivalent Antibodies

In one embodiment, the antibodies of this disclosure may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0129125.

In one embodiment, the antibodies are bispecific antibodies in which the two chains have different specificities. Other embodiments include antibodies with additional specificities, such as tri-specific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to more than one target molecule as well as a heterologous epitope, such as a heterologous polypeptide or solid support material. In some embodiments, the multivalent antibodies may include bispecific antibodies or trispecific antibodies. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge. CH2, and/or CH3 regions, using methods well known to those of ordinary skill in the art.

### Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384 by Ishikawa et al.

The present disclosure also encompasses a human monoclonal antibody described herein conjugated to a therapeutic agent, a polymer, a detectable label or enzyme. In one embodiment, the therapeutic agent is a cytotoxic agent. In one embodiment, the polymer is PEG.

### Nucleic Acids, Expression Cassettes, and Vectors

The present disclosure provides isolated nucleic acid segments that encode the polypeptides, peptide fragments, coupled proteins, antibodies, and protein complexes of this disclosure. The nucleic acid segments of this disclosure also include segments that encode for the same amino acids due to the degeneracy of the genetic code. For example, the amino acid threonine is encoded by ACU, ACC, ACA, and ACG and is therefore degenerate. It is intended that the disclosure includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of this disclosure include both the naturally occurring sequences as well as mutant forms.

The nucleic acid segments of this disclosure may be contained within a vector. A vector nay include, but is not limited to, any plasmid, phagemid, F-factor, virus, cosmid, or phage in a double- or single-stranded linear or circular form which may or may not be self transmissible or mobilizable. The vector can also transform a prokaryotic or eukaryotic host either by integration into the cellular genome or exist extra-chromosomally (e.g., autonomous replicating plasmid with an origin of replication).

Preferably the nucleic acid segment in the vector is under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of this disclosure may also be inserted into an expression vector. Typically, an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment, and the treated vector are combined in the presence of a suitable buffer and ligase. The mixture is then incubated under appropriate conditions to allow the ligase to ligate the nucleic acid fragment into the vector.

The disclosure also provides an expression cassette which contains a nucleic acid sequence capable of directing expression of a particular nucleic acid segment of this disclosure, either in vitro or in a host cell. Also, a nucleic acid segment of this disclosure may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences, which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The disclosure also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of this disclosure into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

In another aspect, this disclosure also provides (i) a nucleic acid molecule or molecules encoding a polypeptide chain of the antibody or antigen-binding fragment thereof or protein complex described herein: (ii) a vector comprising the nucleic acid molecule or molecules as described; and (iii) a cultured host cell comprising the vector as described. Also provided is a method for producing a polypeptide, comprising: (a) obtaining the cultured host cell as described; (b) culturing the cultured host cell in a medium under conditions permitting expression of a polypeptide encoded by the vector and assembling of an antibody or fragment thereof or protein complex; and (c) purifying the antibody or fragment or protein complex from the cultured cell or the medium of the cell.

### Methods of Production

Antibodies or antibody fragments or protein complexes may be produced using recombinant methods and compositions, e.g., as described in U.S. Pat. No. 4,816,567. In one embodiment, an isolated nucleic acid encoding an antibody described herein is provided. Such nucleic acid may encode an amino acid sequence comprising the VL and/or an amino acid sequence comprising the VII of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gemgross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CVI line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR-CHO cells (Urlaub et a. Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### Compositions and Formulations

The antibodies or protein complexes of this disclosure represent an excellent way for the development of therapies either alone or in combination with other therapeutic agents for the treatment of various disorders.

In another aspect, the present disclosure provides a pharmaceutical composition comprising the antibodies or protein complexes of the present disclosure described herein formulated together with a pharmaceutically acceptable carrier. The composition may optionally contain one or more additional pharmaceutically active ingredients, such as another antibody or a therapeutic agent.

The pharmaceutical compositions also can be administered in a combination therapy with, for example, another immune-stimulatory agent, anti-cancer agent, an antiviral agent, or a vaccine, etc. In some embodiments, a composition comprises an antibody or protein complex of this disclosure at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, the second therapeutic agent comprises an anti-inflammatory drug or an antiviral compound. In some embodiments, the antiviral compound comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral compound may include: acyclovir, gancyclovir, vidarabine, foscarnet, cidofovir, amantadine, ribavirin, trifluorothymidine, zidovudine, didanosine, zalcitabine or an interferon. In some embodiments, the interferon is an interferon-α, an interferon-β, an interferon-γ, or an interferon-λ.

Also within the scope of this disclosure is use of the pharmaceutical composition in the preparation of a medicament for the diagnosis, prophylaxis, treatment, or combination thereof of a disease condition, such as cancer or an infection with a pathogen.

The pharmaceutical composition can comprise any number of excipients. Excipients that can be used include carriers, surface-active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, a pharmaceutical composition is suitable for intravenous, intramuscular, subcutaneous, parenteral, spinal or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the present disclosure described herein can be administered via a non-parenteral route, such as a topical, epidermal or mucosal route of administration, e.g., intranasally, orally, vaginally, rectally, sublingually or topically.

The pharmaceutical compositions of this disclosure may be prepared in many forms that include tablets, hard or soft gelatin capsules, aqueous solutions, suspensions, and liposomes and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody or protein complex can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody or protein complex can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, an antibody or protein complex may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, an antibody may be administered via a liquid spray, such as via a plastic bottle atomizer.

Pharmaceutical compositions may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

The pharmaceutical composition of the present disclosure can be in the form of sterile aqueous solutions or dispersions. It can also be formulated in a microemulsion liposome, or other ordered structure suitable to high drug concentration.

An antibody or protein complex of the present disclosure described herein can be administered as a sustained release formulation, in which case less frequent administration is required. Dosage and frequency vary depending on the half-life of the antibody or protein complex in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The amount of active ingredient that can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens can be adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the antibody or protein complex can be administered as a sustained release formulation, in which case less frequent administration is required. For administration of the antibody or protein complex, the dosage ranges from about 0.0001 to 800 mg/kg, and more usually 0.01 to 5 mg/kg, of the host body weight. For example, dosages can be 0.3 mg/kg body weight, 1 mg/kg body weight. 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every 3 to 6 months. Preferred dosage regimens for an antibody of this disclosure include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody or protein complex concentration of about 1-1000 μg/ml and in some methods about 25-300 μg/ml. A “therapeutically effective dosage” of an antibody of this disclosure preferably results in a decrease in severity of disease symptoms, an increase in frequency and duration of disease symptom-free periods, or a prevention of impairment or disability due to the disease affliction.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparati (U.S. Pat. Nos. 4,447,233 and 4,447,224); and (5) osmotic devices (U.S. Pat. Nos. 4,439,196 and 4,475,196); the disclosures of which are incorporated herein by reference.

In some embodiments, the antibodies or protein complexes of this disclosure described herein can be formulated to ensure proper distribution in vivo. For example, to ensure that the therapeutic compounds of this disclosure cross the blood-brain barrier, they can be formulated in liposomes, which may additionally comprise targeting moieties to enhance selective transport to specific cells or organs. See, e.g., U.S. Pat. Nos. 4,522,811; 5,374,548; 5,416,016; and 5,399,331; VV. Ranade (1989) Clin. Pharmacol. 29:685; Umezawa et al., (1988) Biochem. Biophys. Res. Commun, 153:1038; Bloeman et al. (1995) FEBS Lett. 357:140; M. Owais et al. (1995) Antimicrob. Agents Chemother. 39:180; Briscoe et at (1995) Am. Physiol. 1233:134; Schreier et al. (1994). Biol. Chem. 269:9090; Keinanen and Laukkanen (1994) FEBS Lett. 346:123; and Killion and Fidler (1994) Immunomethods 4:273.

In some embodiments, the initial dose may be followed by administration of a second or a plurality of subsequent doses of the antibody or antigen-binding fragment thereof or protein complex in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks: or at least 14 weeks.

Various delivery systems are known and can be used to administer the pharmaceutical composition of this disclosure, e.g., encapsulation in liposomes, microparticles, microcapsules, recombinant cells capable of expressing the mutant viruses, receptor-mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can also be delivered in a vesicle, in particular, a liposome (see, for example, Langer (1990) Science 249: 1527-1533).

The use of nanoparticles to deliver the antibodies or protein complexes of the present disclosure is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389), incorporated herein by reference. Nanoparticles may be developed and conjugated to antibodies or protein complexes contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25m pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P) and the HUMIRA™ Pen (Abbott Labs, Abbott Park, IL), to name only a few.

Advantageously, the pharmaceutical compositions for oral or parenteral use described herein are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 ng per dosage form in a unit dose: especially in the form of injection, it is preferred that the antibody is contained in about 5 to about 300 mg and in about 10 to about 300 mg for the other dosage forms.

### Other Therapeutic Agents

Various other therapeutic agents can be included in the pharmaceutical compositions described above or co-administered with the compositions, simultaneously, before or afterwards. Such a therapeutic agent can also be conjugated to the antibody or incorporated into the protein complex as an effector agent as described herein. Examples of these therapeutic agents include but not limited to cytotoxic agents, anti-angiogenic agents, pro-apoptotic agents, antibiotics, hormones, hormone antagonists, chemokines, drugs, prodrugs, toxins, cytokines, complement agents, checkpoint inhibitors, immune costimulatory/agonist agents, immune coinhibitory/antagonist agents, and enzymes. Drugs of use may possess a pharmaceutical property selected from the group consisting of antimitotic, angiokinase, alkylating, antimetabolite, antibiotic, alkaloid, anti-angiogenic, pro-apoptotic agents and combinations thereof.

Exemplary drugs of use may include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-li), SN-38, carboplatin, cladribine, camptothecins, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temozolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

Toxins of use may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Chemokines of use may include RANTES, MCAF, MIP1-alpha, MIP1-Beta and IP-10.

In certain embodiments, anti-angiogenic agents, such as angiostatin, baculostatin, canstatin, maspin, anti-VEGF antibodies, anti-PIGF peptides and antibodies, anti-vascular growth factor antibodies, anti-Flk-1 antibodies, anti-Fit-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, Gro-β, thrombospondin, 2-methoxyestradiol, proliferin-related protein, carboxyamidotriazole, CM101, Marimastat, pentosan polysulphate, angiopoietin-2, interferon-alpha, herbimycin A, PNU145156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4 or minocycline may be of use.

Immunomodulators of use may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β, -γ or -λ, and stein cell growth factor, such as that designated “S1 factor” Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and LT.

Radionuclides of use include, but not limited to, ¹¹¹In, ¹⁷¹Lu, ²¹²Bi, ²¹²Bi, ²¹³At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶Ho, ¹⁸⁶Re, ¹⁸⁸Re, ¹⁸⁹Re, 212Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²¹¹Pb, and ²²⁷Th. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, I-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 key, more preferably 3,000-8,000 keV, and most preferably 4,000-7,000 keV. Additional potential radioisotopes of use include ¹¹C, ¹³N, ¹⁵O, ⁷⁵Br ¹⁹⁸Au, ²²⁴Ac, ¹²⁶I, ¹³³I, ⁷⁷Br, ^{133m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like. Some useful diagnostic nuclides may include ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ^{94m}Tc, ^{99m}Tc, or ¹¹¹In.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni el al. (eds.), PHOTODYNAMIC THERAPY OF TUMORS AND OTHER DISEASES (Libreria Progetto 1985): van den Bergh, Chem. Britain (1986). 22:430, Moreover, monoclonal antibodies have been coupled with photoactivated dyes for achieving phototherapy. See Mew et al., J. Immunol. (1983), 130:1473; idem., Cancer Res. (1985), 45:4380; Oseroff et al., Proc. Natl. Acad. Sci. USA (1986), 83:8744; idem., Photochem. Photobiol. (1987). 46:83; Hasan et al., Prog. Clin. Biol. Res. (1989), 288:471; Tatsuta et al., Lasers Surg Med. (1989), 9:422; Pelegrin et al., Cancer (1991). 67:2529.

Other useful therapeutic agents may comprise oligonucleotides, especially antisense oligonucleotides that preferably are directed against oncogenes and oncogene products, such as bcl-2 or p53, A preferred form of therapeutic oligonucleotide is siRNA The skilled artisan will realize that any siRNA or interference RNA species may be attached to an antibody or fragment thereof for delivery to a targeted tissue. Many siRNA species against a wide variety of targets are known in the art, and any such known siRNA may be utilized in the claimed methods and compositions.

Known siRNA species of potential use include those specific for IKK-gamma (U.S. Pat. No. 7,022,828); VEGF, Fit-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase 11 (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif), Ambion (Austin, Tex.), Dharmacon (Thermo Scientific, Lafayette, Colo.). Promega (Madison, Wis.), Mirus Bio (Madison, Wis.) and Qiagen (Valencia, Calif), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCB1. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject complexes described herein.

### Methods and Uses

The protein complexes, antibodies, and methods disclosed herein have a wide variety of utilities. As such they have a broad spectrum of applications in, e.g., research, diagnosis, and therapy.

### Methods of Treatment

The protein complexes, antibodies, compositions and formulations described herein can be used to treat various diseases or conditions, including cancer (e.g., breast cancer, lung cancer, gastric cancer, colorectal cancer, bladder cancer, liver cancer, prostate cancer, pancreatic cancer, melanoma, leukemia, lymphoma, multiple myeloma), an immunological disease (e.g., autoimmune diseases) and an infection with a pathogen (such as a virus, a bacterium, a fungus, or parasite). Accordingly, various embodiments concern methods of treating such a disease or condition in a subject in need thereof, comprising administering to the subject a therapeutically effective amount of a protein complex, antibody, composition, or formulation described herein.

In one embodiment, immunological diseases which may be treated with the protein complex or antibody may include, for example, autoimmune disease such as systemic lupus erythematosus (SLE), joint diseases such as ankylosing spondylitis, juvenile rheumatoid arthritis, rheumatoid arthritis; neurological disease such as multiple sclerosis and myasthenia gravis; pancreatic disease such as diabetes, especially juvenile onset diabetes; gastrointestinal tract disease such as chronic active hepatitis, celiac disease, ulcerative colitis, Crohn's disease, pernicious anemia; skin diseases such as psoriasis or scleroderma; allergic diseases such as asthma and in transplantation related conditions such as graft versus host disease and allograft rejection.

The administration of the protein complex or antibody can be supplemented by administering concurrently or sequentially a therapeutically effective amount of another antibody that binds to or is reactive with another antigen on the surface of the target cell. Preferred additional MAbs comprise at least one humanized, chimeric or human MAb selected from the group consisting of a MAb reactive with CD4, CD5, CD8, CD14, CD15, CD16, CD19, IGF-1R, CD20, CD21, CD22, CD23, CD25, CD27, CD30, CD32b, CD33, CD37, CD38, CD40, CD40L, CD45. CD46, CD47, CD52, CD54, CD66, CD70, CD74, CD79a, CD79b, CD80, CD95, CD126, CD133, CD138, CD154, CD160, CD166, CD229, CEACAM5, CEACAM6, B7, AFP, PSMA, EGP-1, EGP-2, GPRC5, FcRH5, ROR1, BCMA, IF-1R, EGFR, HER2, HER3, TNE-α, ICOS, ICOSL, hypoxia inducible factor (HIF), folate receptor, TDGF1, TfR, Mesothelin, PSMA, B7, TFN-α, TFN-β, IFN-γ, IFN-λ, C1r, C1s, C2, C3, C5, C5a, C5aR1, C6, MASPs, MSAP2, MASP3, FB, FD, Properdin, Lag-3, CTLA-4, PD-1, PD-L1, TIM3, SIRPα, TIGIT, OX40, OX40L, 4-1BB, BTLA, GITR, GITRL, TCR, Nectin-4, c-Met, LIV1, Mesothelin, DLL3, DLL4, Tissue factor, TGE-β, TGE-β receptor, CLDN18.2, carbonic anhydrase IX, PAM4 antigen, MUC1, MUC2, MUC3, MUC4, MUC5, MUC16, 1a, MIF, HM124, HLA-DR, tenascin, Flt-3, VEGFR, PIGF, ILGF, IL-1β, IL2, IL-6, IL-6R, IL-15, IL-15R, IL-17, IL-17R, IL-12, IL-25, tenascin, TRAIL-R1, TRAIL-R2, complement factor C5, oncogene product, or a combination thereof. Various antibodies of use, such as anti-CD19, anti-CD20, and anti-CD22 antibodies, are known to those of skill in the art. See, for example, (Ghetie et al., Cancer Res. 48:2610 (1988); Heiman et al., Cancer Immunol. Immunother. 32:364 (1991); Longo, Curr. Opin. Oncol. 8:353 (1996), U.S. Pat. Nos. 5,798,554; 6,187,287; 6,306,393; 6,676,924; 7,109,304; 7,151,164; 7,230,084; 7,230,085; 7,238,785; 7,238,786; 7,282,567; 7,300,655; 7,312,318; 7,501,498; 7,612,180; 7,670,804; and U.S. Patent Application Publ. Nos. 20080131363; 20070172920; 20060193865; and 20080138333, the Examples section of each incorporated herein by reference.

The therapy can be further supplemented with the administration, either concurrently or sequentially, of at least one therapeutic agent. For example, “CVB” (1.5 g/m² cyclophosphamide, 200-400 mg/m² etoposide, and 150-200 mg/m² carmustine) is a regimen used to treat non-Hodgkin's lymphoma. Patti et al., Eur. J. Haematol. 51: 18 (1993). Other suitable combination chemotherapeutic regimens are well-known to those of skill in the art. See, for example, Freedman el al., “Non-Hodgkin's Lymphomas,” in CANCER MEDICINE, VOLUME 2, 3rd Edition, Holland et al. (eds), pages 2028-2068 (Lea & Febiger 1993). As an illustration, first generation chemotherapeutic regimens for treatment of intermediate-grade non-Hodgkin's lymphoma (NHL) include C-MOPP (cyclophosphamide, vincristine, procarbazine and prednisone) and CHOP (cyclophosphamide, doxorubicin, vincristine, and prednisone). A useful second-generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate, bendamustine, and bryostatin-1.

The subject protein complex or antibody can be formulated according to known methods to prepare pharmaceutically useful compositions, whereby the bsAb is combined in a mixture with a pharmaceutically suitable excipient. Sterile phosphate-buffered saline is one example of a pharmaceutically suitable excipient. Other suitable excipients are well-known to those in the art. See, for example, Ansel et al, PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The subject protein complex or antibody can be formulated for intravenous administration via, for example, bolus injection or continuous infusion. Preferably, the protein complex or antibody is infused over a period of less than about 4 hours, and more preferably, over a period of less than about 3 hours. For example, the first bolus could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Additional pharmaceutical methods may be employed to control the duration of action of the protein complex or antibody. Control release preparations can be prepared through the use of polymers to complex or adsorb the protein complex or antibody. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release from such a matrix depends upon the molecular weight of the protein complex or antibody, the amount of protein complex or antibody within the matrix, and the size of dispersed particles. Saltzman et al., Biophys. J 55: 163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The protein complex or antibody may also be administered to a mammal subcutaneously or even by other parenteral routes, such as intravenously, intramuscularly, intraperitoneally or intravascularly. Moreover, the administration may be by continuous infusion or by single or multiple boluses. Preferably, the protein complex or antibody is infused over a period of less than about 4 hours, and more preferably, over a period of less than about 3 hours.

More generally, the dosage of an administered protein complex or antibody for humans will vary depending upon such factors as the patient's age, weight, height, sex, general medical condition and previous medical history. It may be desirable to provide the recipient with a dosage of protein complex or antibody that is in the range of from about 1 mg/kg to 25 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy.

Alternatively, a protein complex or antibody may be administered as one dosage every 2 or 3 weeks, repeated for a total of at least 3 dosages. Or, the construct may be administered twice per week for 4-6 weeks. If the dosage is lowered to approximately 200-300 mg/m² 340 mg per dosage for a 1.7-m patient, or 4.9 mg/kg for a 70 kg patient), It may be administered once or even twice weekly for 4 to 10 weeks. Alternatively, the dosage schedule may be decreased, namely every 2 or 3 weeks for 2-3 months. It has been determined, however, that even higher doses, such as 20 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

While the protein complex or antibody may be administered as a periodic bolus injection, in alternative embodiments the bs protein complex or antibody Abs may be administered by continuous infusion. In order to increase the Cmax and extend the PK of the protein complex or antibody in the blood, a continuous infusion may be administered for example by indwelling catheter. Such devices are known in the art, such as HICKMAN®, BROVIAC® or PORT-A-CATH® catheters (see, e.g., Skolnik et al., Ther Drug Monit 32:741-48, 2010) and any such known indwelling catheter may be used. A variety of continuous infusion pumps are also known in the art and any such known infusion pump may be used. The dosage range for continuous infusion may be between 0.1 and 3.0 mg/kg per day. More preferably, the protein complex or antibody can be administered by intravenous infusions over relatively short periods of 2 to 5 hours, more preferably 2-3 hours.

In preferred embodiments, the protein complex or antibody are of use for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial cancer or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Cancers conducive to treatment methods of the present invention involves cells which express, over-express, or abnormally express IGF-1R.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia. Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma. Adult Primary Liver Cancer, Adult Soft Tissue Sarcoma, AIDS-Related Lymphoma, AIDS-Related Malignancies. Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stern Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational TROPhoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer. Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, TROPhoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, BASIC PATHOLOGY, 2d Ed., W. B. Saunders Co., Philadelphia, pp. 68-79 (1976)). Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be treated include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, peiapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis.

In preferred embodiments, the method of the disclosure is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

### Diagnostic Uses

The antibodies or protein complexes described herein may be used to detect and/or measure an antigen in a sample, e.g., for diagnostic purposes. Accordingly, some embodiments contemplate the use of one or more antibodies or protein complexes described herein in assays to detect the antigen and associated-disease or disorder.

Exemplary diagnostic assays may comprise, e.g., contacting a sample, obtained from a patient, with an antibody or protein complex of this disclosure, wherein the antibody or protein complex is labeled with a detectable label or reporter molecule or used as a capture ligand to selectively isolate the antigen from a sample. In some embodiments, detectable label or reporter molecule is incorporate into the protein complex as an effector agent. Alternatively, an unlabeled antibody or protein complex can be used in diagnostic applications in combination with a secondary antibody, which is itself detectably labeled.

The detectable label or reporter molecule can be a radioisotope, such as H, C, P, S, or I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure a target in a sample include enzyme-linked immunosorbent assay (ELISA), radioimmunoassay (RIA), and fluorescence-activated cell sorting (FACS).

In another aspect, this disclosure further provides a method for detecting the presence of an antigen in a sample comprising the steps of: (i) contacting a sample with the antibody or antigen-binding fragment or protein complex described herein; and (ii) determining binding of the antibody or antigen-binding fragment or protein complex to the antigen, wherein binding of the antibody to the antigens is indicative of the antigen in the sample and associated-disease or disorder.

In some embodiments, the anti body or antigen-binding fragment or protein complex is conjugated to a label. In some embodiments, the step of detecting comprises contacting a secondary antibody with the antibody or antigen-binding fragment thereof and wherein the secondary antibody comprises a label. In some embodiments, the label includes a fluorescent label, a chemiluminescent label, a radiolabel, and an enzyme.

In some embodiments, the step of detecting comprises detecting fluorescence or chemiluminescence. In some embodiments, the step of detecting comprises a competitive binding assay or ELISA.

In some embodiments, the method further comprises binding the sample to a solid support. In some embodiments, the solid support includes microparticles, microbeads, magnetic beads, and an affinity purification column.

Samples that can be used in diagnostic assays according to the present disclosure include any tissue or fluid sample obtainable from a patient, which contains detectable quantities of an antigen of interest under normal or pathological conditions. Generally, levels of the antigen in a particular sample obtained from a healthy patient (e.g., a patient not afflicted with a disease associated with the antigen) will be measured to initially establish a baseline, or standard, level of the antigen. This baseline level can then be compared against the levels measured in samples obtained from individuals suspected of having the antigen and associated condition, or symptoms associated with such condition.

### Kits

In another aspect, this disclosure provides a kit comprising a pharmaceutically acceptable dose unit of the antibody or antigen-binding fragment thereof of or the protein complex, or the pharmaceutical composition as described herein. Also within the scope of this disclosure is a kit for the diagnosis, prognosis or monitoring the treatment of a disorder in a subject, comprising: the antibody or antigen-binding fragment thereof of or the protein complex as described; and a least one detection reagent that binds specifically to the antibody or antigen-binding fragment of or the protein complex thereof.

In some embodiments, the kit also includes a container that contains the composition and optionally informational material. The informational material can be descriptive, instructional, marketing or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody or protein complex contained within the kit into an animal, such as a human.

### Definitions

A nucleic acid or polynucleotide refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. The terms also apply to amino acid polymers in which one or more amino acid residues is an analog or mimetic of a corresponding naturally occurring amino acid, as well as to naturally occurring amino acid polymers. Polypeptides and proteins can be produced by a naturally-occurring and non-recombinant cell: or it is produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass antigen binding proteins, antibodies, or sequences that have deletions from, additions to, and/or substitutions of one or more amino acids of an antigen-binding protein.

The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8, 9, 10, 11, 12, 13, 14, 15, 20, 50, 70, 100, 110, 150, 200, 250, 300, 350, 400, or 450 amino acids long. Useful polypeptide fragments include immunologically functional fragments of antibodies, including binding domains.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” means any molecule or entity (e.g., nucleic acid, plasmid, bacteriophage or virus) used to transfer protein coding information into a host cell. In some embodiments, a “vector” refers to a delivery vehicle that (a) promotes the expression of a polypeptide-encoding nucleic acid sequence; (b) promotes the production of the polypeptide therefrom: (c) promotes the transfection/transformation of target cells therewith; (d) promotes the replication of the nucleic acid sequence: (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid, A vector can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

The term “expression vector” or “expression construct” refers to a vector that is suitable for transformation of a host cell and contains nucleic acid sequences that direct and/or control (in conjunction with the host cell expression of one or more heterologous coding regions operatively linked thereto. An expression construct may include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed with a nucleic acid sequence and thereby expresses a gene of interest. The term includes the progeny of the parent cell, whether or not the progeny is identical in morphology or in genetic make-up to the original parent cell, so long as the gene of interest is present.

The term “immune cells” refers to cells of hematopoietic origin that are involved in the specific recognition of antigens. Immune cells include antigen presenting cells (APCs), such as dendritic cells or macrophages, B cells, T-cells, NIK cells such as NK-92 cells, etc. T-cells include Teff cells and Treg cells.

By “effector cell” as used herein is meant a cell of the immune system that has been induced to differentiate into a form capable of mounting a specific immune response, or a cell that expresses one or more Fc receptors and mediates one or more effector functions. Effector cells include but are not limited to monocytes, macrophages, neutrophils, dendritic cells, eosinophils, mast cells, platelets, T cells, B cells, large granular lymphocytes, Langerhans' cells, natural killer (NK) cells, and γδ T cells, and may be from any organism including but not limited to humans, mice, rats, rabbits, and monkeys. By “effector function” as used herein is meant a biochemical event that results from the interaction of an antibody Fc region with an Fc receptor or ligand. Effector functions include FcγR-mediated effector functions such as ADCC and ADCP, and complement-mediated effector functions such as CDC.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., a targeting domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities, A linker can be a covalent linker or a non-covalent linker, Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the targeting domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The terms “subject” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed. In some embodiments, a subject may be an invertebrate animal, for example, an insect or a nematode; while in others, a subject may be a plant or a fungus.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “variant” refers to a second composition (e.g., a second molecule), that is related to a first composition (e.g. a first molecule, also termed a “parent” molecule). The variant molecule can be derived from, isolated from, based on or homologous to the parent molecule. The term variant can be used to describe either polynucleotides or polypeptides.

As applied to polypeptide or protein, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

A functional variant or equivalent of a reference peptide, polypeptide, or protein refers to a polypeptide derivative of the reference peptide, polypeptide, or protein, e.g., a protein having one or more point mutations, insertions, deletions, truncations, a fusion protein, or a combination thereof. It retains substantially the activity to of the reference peptide, polypeptide, or protein. In general, the functional equivalent is at least 50% (e.g., any number between 50% and 100%, inclusive, e.g., 60%, 70%, 80%, 85%, 90%, 95%, and 99%) identical to the reference peptide, polypeptide, or protein.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the disclosure.

In another aspect, polypeptide variants include polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. One of skill will appreciate that many variants of the disclosed polypeptides are encompassed by the disclosure. In some aspects, polynucleotide or polypeptide variants of the disclosure can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where a substitution within a group is a “conservative” amino acid substitution. This grouping indicated below is not rigid, as these natural amino acids can be placed in different grouping when different functional properties are considered. Amino acids having nonpolar and/or aliphatic side chains include: glycine, alanine, valine, leucine, isoleucine and proline. Amino acids having polar, uncharged side chains include: serine, threonine, cysteine, methionine, asparagine and glutamine. Amino acids having aromatic side chains include: phenylalanine, tyrosine and tryptophan. Amino acids having positively charged side chains include: lysine, arginine and histidine. Amino acids having negatively charged side chains include: aspartate and glutamate.

As applied to polynucleotides, a variant molecule can have entire nucleotide sequence identity with the original parent molecule, or alternatively, can have less than 100% nucleotide sequence identity with the parent molecule. For example, a variant of a nucleotide sequence can be a second nucleotide sequence that is at least 50%, 60%, 70%, 80%, 90%, 95%, 98%, 99% or more identical in nucleotide sequence compared to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the disclosure.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the disclosure. One of skill will appreciate that many variants of the disclosed nucleotide sequences are encompassed by the disclosure.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither, or both limits are included in the smaller ranges is also encompassed within the disclosure, subject to any specifically excluded limit in the stated range. Where the stated range includes one or both of the limits, ranges excluding either or both of those included limits are also included in the disclosure. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3 Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An “immunoglobulin (Ig)” is meant a protein consisting of one or more polypeptides substantially encoded by immunoglobulin genes. Immunoglobulins include but are not limited to antibodies. Immunoglobulins may have a number of structural forms, including but not limited to full length antibodies, antibody fragments, and individual immunoglobulin domains.

The term “antigen-binding fragment or portion” of an antibody (or simply “antibody fragment or portion”), as used herein, refers to one or more fragments of an antibody that retain the ability to specifically bind to an antigen (e.g., CD3). It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)2 fragment, a bivalent fragment comprising two Fab fragments linked by a disulfide bridge at the hinge region: (iii) a Fab′ fragment, which is essentially an Fab with part of the hinge region (see, FUNDAMENTAL IMMUNOLOGY (Paul ed., 3Y ed. 1993)); (iv) a Fd fragment consisting of the V_{H} and C_{H}I domains; (v) a Fv fragment consisting of the V_{L} and VH domains of a single arm of an antibody, (vi) a dAb fragment (Ward el al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv), see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

By “constant region” of an antibody as defined herein is meant the region of the antibody that is encoded by one of the light or heavy chain immunoglobulin constant region genes. By “constant light chain” or “light chain constant region” as used herein is meant the region of an antibody encoded by the kappa (CK) or lambda (Ck) light chains. The constant light chain typically comprises a single domain, and as defined herein refers to positions 108-214 of Cκ or Cλ, wherein numbering is according to the EU index. By “constant heavy chain” or “heavy chain constant region” as used herein is meant the region of an antibody encoded by the imu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant the polypeptides that comprise the V_{H}, CH1, V_{H}, and C_{L} immunoglobulin domains. Fab may refer to this region in isolation, or this region in the context of a full length antibody or antibody fragment.

By “Fe” or “Fc region” or “Fc domain”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, for purpose herein, the human IgG heavy chain Fc region is defined as starting at E216 to its carboxyl-terminus, wherein the numbering is according to the EU index as in Kabat. Fc may refer to this region in isolation, or this region in the context of an Fc polypeptide such as an antibody or immunoadhesin (e.g. an Fc fusion protein), as described below. It should be noted that for the purposes described herein, “Fc region” generally includes the hinge region, comprising residues 216-237, unless noted otherwise. Thus, an “Fc variant” can include variants of the hinge region, in the presence or absence of additional amino acid modifications in the Cγ2 and Cγ3 domains.

By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 215, and the IgG CH2 domain begins at residue EU position 238. Thus for IgG the antibody hinge is herein defined to include positions 216 (E216 in IgG1) to 237 (G237 in IgG1), wherein the numbering is according to the EU index as in Kabat. In some embodiments, for example in the context of an Fc region, the lower hinge is included, with the “lower hinge” generally referring to positions 231 to 237.

An “isolated antibody”, as used herein, is intended to refer to an antibody that is substantially free of other antibodies having different antigenic specificities (e.g. an isolated antibody that specifically binds to a specific antigen, is substantially free of antibodies that specifically bind antigens other than the specific antigen). An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody. The term can also refer to an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody).

“Single chain antibodies” or “scFvs” are Fv molecules in which the heavy and light chain variable regions have been connected by a flexible linker to form a single polypeptide chain, which forms an antigen-binding region. scFvs are discussed in detail in WO 88/01649 and U.S. Pat. Nos. 4,946,778 and 5,260,203, the disclosures of which are incorporated by reference.

A “domain antibody” or “single chain immunoglobulin” is an immunologically functional immunoglobulin fragment containing only the variable region of a heavy chain or the variable region of a light chain. Examples of domain antibodies include Nanobodies™, In some instances, two or more VH regions are covalently joined with a peptide linker to create a bivalent domain antibody. The two VH regions of a bivalent domain antibody may target the same or different antigens.

As used herein, the term “antibody fusion protein” is a recombinantly produced antigen-binding molecule in which an antibody or antibody fragment is linked to another protein or peptide, such as the same or different antibody or antibody fragment or a DDD or AD peptide. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase. A preferred immunomodulator might be an interferon, such as interferon-alpha., interferon-beta, or interferon-lamda.

A “molecular complex” is a group of two or more associated moieties or molecules, linked by either covalent or non-covalent interactions. Examples of such a molecular complex include an antibody and an antigen-binding portion thereof.

A “multispecific” complex, protein, or antibody is a complex, protein, or antibody that can bind simultaneously to at least two targets that are of different structure, e.g., two different antigens, two different epitopes on the same antigen, or a hapten and/or an antigen or epitope.

A “multivalent” complex, protein, or antibody is a complex, protein, or antibody that can bind simultaneously to at least two targets that are of the same or different structure. Valency indicates how many binding arms or sites the complex, protein, or antibody has to a single antigen or epitope; i.e., monovalent, bivalent, trivalent or multivalent. The multivalency of the complex, protein, or antibody means that it can take advantage of multiple interactions in binding to an antigen, thus increasing the avidity of binding to the antigen. Specificity indicates how many antigens or epitopes a complex, protein, or antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity.

A “multispecific” complex, protein, or antibody is a complex, protein, or antibody can bind simultaneously to more than one target or epitope which are of different structure, including but not limited to a “bispecific” complex, protein, or antibody. Such a complex, protein, or antibody includes two or more binding moieties with different specificities.

A “bispecific” complex, protein, or antibody is a complex, protein, or antibody that can bind simultaneously to two targets or epitopes which are of different structure. T cell-redirecting bispecific antibodies (bsAb) and bispecific antibody fragments (bsFab or others) may have at least one arm that specifically binds to, for example, a T cell, and at least one other arm that specifically binds to an antigen produced by or associated with a diseased cell, tissue, organ or pathogen, for example a tumor-associated antigen. A variety of bispecific antibodies can be produced using molecular engineering.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

As used herein, a protein that “specifically binds to” an antigen refers to a protein that binds to the antigen when the dissociation constant (KD) is ≤10⁻⁶ M as measured via a surface plasma resonance technique (e.g., BIACore, GE-Healthcare Uppsala, Sweden) or Kinetic Exclusion Assay (KinExA, Sapidyne, Boise, Id.). Preferably, the protein (e.g., antibody) binds to the antigen with “high affinity”, namely with a KD of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less. The term “does not substantially bind” to a protein or cells, as used herein, means does not bind or does not bind with a high affinity to the protein or cells, i.e., binds to the protein or cells with a KD of 1×10⁻⁶ M or more, more preferably 1×10−3 M or more, more preferably 1×10⁻⁴ M or more, more preferably 1×10⁻³ M or more, even more preferably 1×10⁻² M or more.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a Biacore® system.

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from an antigen protein are tested for reactivity with a given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binding pair” refers to a first molecule and a second molecule that specifically bind to each other. Exemplary binding pairs include any haptenic or antigenic compound in combination with a corresponding antibody or binding portion or fragment thereof (e.g., digoxigenin and anti-digoxigenin) and nonimmunological binding pairs (e.g., biotin-avidin, biotin-streptavidin, biotin-neutravidin, hormone (e.g., thyroxine and cortisol-hormone binding protein), receptor-receptor agonist, receptor-receptor antagonist (e.g., acetylcholine receptor-acetylcholine or an analog thereof), IgG-protein A, IgG-protein G, IgG-synthesized protein AG, lectin-carbohydrate, enzyme-enzyme cofactor, enzyme-enzyme inhibitor, and complementary oligonucleotide pairs capable of forming nucleic acid duplexes), and the like. The binding pair can also include a first molecule which is negatively charged and a second molecule which is positively charged.

As used herein, the term “immune response” refers to a biological response within a vertebrate against foreign agents, cancerous or other abnormal cells, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with the disease state. In some embodiments, the effective amount is a therapeutically effective amount or a prophylactically effective amount. When applied to an individual active ingredient, administered alone, the term refers to that ingredient alone. When applied to a combination, the term refers to combined amounts of the active ingredients that result in the therapeutic effect, whether administered in combination, serially or simultaneously.

A “therapeutically effective amount” is an amount sufficient to remedy a disease state or symptoms, particularly a state or symptoms associated with the disease state, or otherwise prevent, hinder, retard or reverse the progression of the disease state or any other undesirable symptom associated with the disease in any way whatsoever.

A “prophylactically effective amount” is an amount of a pharmaceutical composition that, when administered to a subject, will have the intended prophylactic effect, e.g., preventing or delaying the onset (or reoccurrence) of the disease state, or reducing the likelihood of the onset (or reoccurrence) of the disease state or associated symptoms. The full therapeutic or prophylactic effect does not necessarily occur by administration of one dose, and may occur only after administration of a series of doses. Thus, a therapeutically or prophylactically effective amount may be administered in one or more administrations.

As used herein, the term “pharmaceutically acceptable carrier or excipient” refers to a carrier medium or an excipient which does not interfere with the effectiveness of the biological activity of the active ingredient(s) of the composition and which is not excessively toxic to the host at the concentrations at which it is administered. In the context of the present invention, a pharmaceutically acceptable carrier or excipient is preferably suitable for topical formulation. The term includes, but is not limited to, a solvent, a stabilizer, a solubilizer, a tonicity enhancing agent, a structure-forming agent, a suspending agent, a dispersing agent, a chelating agent, an emulsifying agent, an anti-foaming agent, an ointment base, an emollient, a skin protecting agent, a gel-forming agent, a thickening agent, a pH adjusting agent, a preservative, a penetration enhancer, a complexing agent, a lubricant, a demulcent, a viscosity enhancer, a bioadhesive polymer, or a combination thereof. The use of such agents for the formulation of pharmaceutically active substances is well known in the art (see, for example, “Remington's Pharmaceutical Sciences”, E. W. Martin, 18th Ed, 1990 Mack Publishing Co.: Easton, PA which is incorporated herein by reference in its entirety).

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The following examples illustrate the disclosure. These examples should not be construed as to limit the scope of this invention. The examples are included for purposes of illustration, and the present application establishes a priority date for prospective worldwide patent rights covering the disclosure.

### EXAMPLES

### Example 1—Generation of Anti-Trop2 Antibodies

### Methods

### Immunization of Mice

Mice were immunized using a recombinant human Trop2 extracellular fragment (AAI-275). Five mice received 4 consecutive immunizations. Blood was taken and serum prepared, which was used for titer determination by ELISA. Animals with highest titers were selected for boosting, and monoclonal antibodies were isolated by hybridoma technology-fusion of splenocytes to mouse Sp2/0 myeloma cells.

### Determination of Serum Titers (ELISA)

Recombinant human Trop2 extracellular fragment was immobilized on a 96-well Maxisorp plate at 2 μg/ml, 100 ul/well, in PBS, followed by: blocking of the plate with 1% BSA in PBS, 200 ul/well; application of serial dilutions of antisera, in duplicates, 100 ul/well; detection with HRP-conjugated goat anti-mouse antibody (A0168, Sigma). Signal was developed by addition of soluble TMB Substrate, 100 ul/well, 37° C. 10 min, stopped with Sulfuric Acid to obtain a stable yellow color. Absorbance was read out at 450 nm.

### Characterization Anti-Trop2 Antibodies

ELISA for Anti-Trop2 binding to human Trop2: Nunc Maxisorp plates were coated with recombinant human Trop2 extracellular fragment at 5 μg/ml, 100 ul/well, and incubated over night at 4° C. After blocking with Blocker-m Casein in PBS, serial dilutions of purified anti-Trop2 antibodies (start from 0.4 μg/ml, 1:4) were applied to the plates. Bindings were detected with HRP-conjugated goat anti-mouse Fc antibody (Jackson ImmunoResearch, code 115-035-008). Signal was developed by addition of soluble TMB Substrate, 100 ul/well, 37° C. 10 min. Absorbance was read out at 650 nm.

### Flow Cytometry

Cells were trypsinized briefly, washed, re-suspended in 1% BSA-PBS, incubated with anti-Trop2 antibodies, and detected with AF488-labeled goat anti-mouse IgG Fe. All incubations were 45 min at 4° C. with 1% BSA in PBS washes between incubations, Binding signal was measured by Attune™ N×T Acoustic Focusing Cytometer (ThermoFisher Scientific).

### Results

Balb/c Mice were well immunized with recombinant human Trop2 extracellular fragment. After 4 consecutive immunizations, all sera from 5 mice showed significantly high titers when compared with that of pre-immune sera (Table 1). Two animals with highest titers were selected for further boosting prior to fusion. Afterwards, lymphocytes were harvested from mouse spleens and fused with mouse Sp2/0 myeloma cells. Fused cells were incubated in selection media at 37° C./5% CO₂ until large colonies appeared.

TABLE 1
Serum titers from pre- and post-immune mice

| | | Serum | | | | | | | | | |
|---|---|---|---|---|---|---|---|---|---|---|---|
| | | Post-immune serum | | | | | Pre-immune serum | | | | |
| Mice number | | 1 | 2 | 3 | 4 | 5 | 1 | 2 | 3 | 4 | 5 |
| Dilution | 1:10³ | 2.268 | 2.238 | 2.062 | 2.210 | 2.224 | 0.199 | 0.196 | 0.197 | 0.195 | 0.198 |
| | | 2.171 | 2.197 | 2.210 | 2.092 | 2.113 | 0.197 | 0.198 | 0.195 | 0.199 | 0.194 |
| | 1:10⁴ | 1.664 | 1.607 | 1.388 | 1.200 | 1.361 | 0.098 | 0.099 | 0.095 | 0.096 | 0.097 |
| | | 1.721 | 1.572 | 1.446 | 1.397 | 1.656 | 0.095 | 0.094 | 0.097 | 0.098 | 0.096 |
| | 1:10⁵ | 0.891 | 0.852 | 0.743 | 0.642 | 0.752 | 0.044 | 0.042 | 0.041 | 0.043 | 0.045 |
| | | 0.886 | 0.844 | 0.751 | 0.638 | 0.755 | 0.045 | 0.040 | 0.043 | 0.042 | 0.041 |

Supernatants from large colonies were screened by ELISA against human Trop2 captured in Maxisorp plate, and 40 positive supernatants were selected for further screening. Clones were tested for binding to recombinant Trop2 protein (amino acid residues 1-275) by ELISA and binding to Trop2 positive and negative cell lines by FACS. Based on binding affinity and specificity, the clone L0125 was selected for subcloning and cDNA sequencing to produce recombinant and humanized IgG.

### Example 2—Humanization and Characterization of Anti-Trop2 Antibody L0125

### Humanization of Murine Anti-Trop2 Antibody L0125

The amino acid and DNA sequences pertaining to the variable light (W) region were provided in FIG. 2A, and the amino acid and DNA sequences pertaining to the variable heavy (V_{H}) region were provided in FIG. 2B, respectively. The CDRs of L0125 are further delineated in FIG. 2C.

Based upon the amino acid (sequence ID NOs 1 and 3) and DNA (sequence ID NOs 2 and 4) sequences of the V_{L} and V_{H} domains of murine anti-L0125 antibody, chimeric and humanized anti-anti-Trop2 antibodies (cL0125 and hL0125) were generated. The humanization is based on sequence alignment using IGBLAST-A tool for immunoglobulin (IG) and T cell receptor (TR) V domain sequences and BLAST query for human protein database as well as the Therapeutic Antibody Database. The amino acid and DNA sequences of humanized V_{L}-variant are in SEQ ID NOs 5 and 6 respectively, and the amino acid and DNA sequences of humanized V_{H}-variant are in SEQ ID NOs 7 and 8, respectively.

The humanized amino acid sequences for light and heavy chain variable regions of hL0125 were back translated into DNAs and the resulting DNAs were synthesized and cloned into human IgG expression vectors as fusion proteins. The recombinant IgG antibodies were produced and purified from cell culture supernatants by standard methods for antibody preparation.

Characterization of Immunized Anti-Trop2 Antibody hL0125

### SDS-PAGE

SDS-PAGE was performed to examine the purity of hL0125 IgG under reducing and non-reducing conditions using 4-20% Tris-Glycine gels. After resolution, the gel was stained with SimplyBlue™ SafeStain (Thermo Fisher Scientific).

### ELISA

Nunc Maxisorp plates were coated with recombinant human Trop2 extracellular fragment at 5 μg/ml, 100 ul/well, and incubated over night at 4° C. After blocking with Blocker™ Casein in PBS, serial dilutions of purified anti-Trop2 antibodies (start from 0.4 μg/ml, 1:4) were applied to the plates. Bindings were detected with HRP-conjugated goat anti-human Fc antibody (Jackson ImmunoResearch, code 115-035-008). Signal was developed by addition of TMB Substrate soluble, 100 ul/well, 37° C. 10 min. Absorbance was read out at 650 nm.

### Flow Cytometry

Cells were trypsinized briefly, washed, re-suspended in 1% BSA-PBS, incubated with cL0125 and hL0125, and detected with AF488-labeled goat anti-human IgG Fc. All incubations were 45 min at 4° C. plus washes with 1% BSA in PBS between incubations. Binding signal was measured by Attune™ N×T Acoustic Focusing Cytometer (ThermoFisher Scientific).

### Results

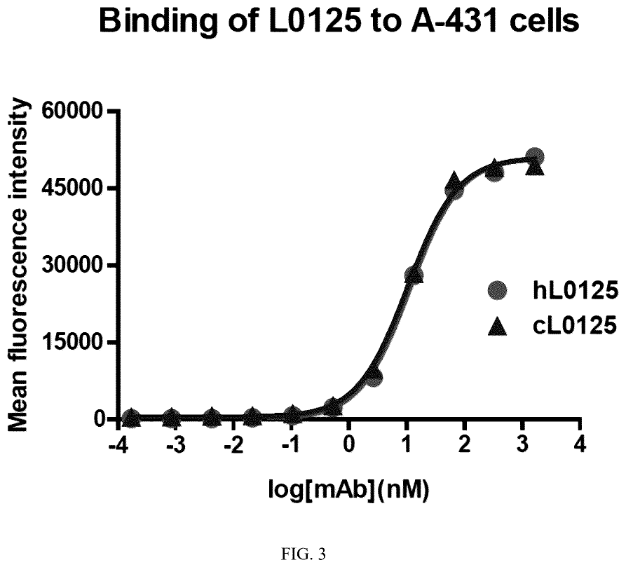

The amino acid and DNA sequences pertaining to the V_{L} and V_{H} of L0125 were provided in FIG. 2A and FIG. 2B, respectively. The CDRs of L0125 are further delineated in FIG. 2C. The amino acid and DNA sequences pertaining to the V_{L} and V_{H} of hL0125 were provided in FIG. 2D aid FIG. 2E, respectively. The cL0125 and hL0125 MAbs were produced and purified to homogeneity, as shown by SE-HPLC and SDS-PAGE analysis (data not shown). Their bioactivities were confirmed and compared by flow cytometry showing the same binding affinity to Trop2-positive A-431 cells (FIG. 3).

### Example 3—Generation of Cell Line Expressing Anti-CD3 Single Chain-AD2

To make T-cell redirecting IgG-scFv bispecific antibodies, a master cell line was first generated expressing an anti-CD3-scFv-AD2 module. The module was designed from humanized Okt3 mAb against CD3 with addition of an anchor domain (AD2) from AKAP protein and assembled in the format of V_{H}-L1-V_{K}-L2-AD2-GS-6H, where the V domains of humanized Okt3 mAb were fused via a flexible peptide linker, followed by AD2 and a 6-His tag. The sequences of the leader peptide, anti-CD3 variable domains, linkers, and AD2 were shown below.

Leader peptide
(SEQ ID NO 9)
MGWSCIILFLVATATGVHS
V_{H} sequence of anti-CD3 scFv
(SEQ ID NO 10)
DIKLQQSGAELARPGASVKMSCKTSGYTFTRYTMHWVKQRP
GQGLEWIGYINPSRGYTNYNQKFKDKATLTTDKSSSTAYMQ
LSSLTSEDSAVYYCARYYDDHYSLDYWGQGTTLTVSS
11 linker
(SEQ ID NO 11)
VEGGSGGSGGSGGSGGVD
V_{K} sequence of anti-CD3 scEv
(SEQ ID NO 12)
DIQLTQSPAIMSASPGEKVTMTCRASSSVSYMNWYQQKSG
TSPKRWIYDTSKVASGVPYRESGSGSGTSYSLTISSMEAE
DAATYYCQQWSSNPLTFGAGTKLELK
L2 linker
(SEQ ID NO 13)
GGGSGGGSGGGS
AD2 peptide
(SEQ ID NO 14)
CGQIEYLAKQIVDNAIQQAGC
GS-6-His
(SEQ ID NO 15)
GS-HHHHHH

A cDNA sequence encoding the leader peptide and anti-CD3-scFv-AD2 (SEQ ID NOs 9-15) was synthesized and cloned into the pcDNA3.1(+) or pcDNA3.4 vector. A Kozak sequence of “GCCGCCACC” (SEQ ID NO:55) was added adjacent to the ATG start codon to produce the final expression vector. The expression vector was transfected into CHO or Sp2/0 cells using the Neon Electroporation Transfection System. Clones were selected in media containing 0.25 mg/ml G418 and screened for protein expression by dot blot. The anti-CD3-scFv-AD2 was captured on nitrocellulose membranes and detected with an HRP-labeled anti-His mAb. The clone with the highest protein expression was further cultured and screened until a stable subclone was established as a master cell line. The anti-CD3-scFv-AD 2 module was designated as 3scFv (SEQ ID NO 33). The master cell line was designated as 3scFv-C21.

(SEQ ID NO 33)

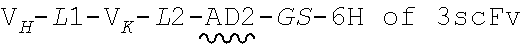

DIKLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYYDD
HYSLDYWGQGTTLTVSS-
VEGGSGGSGGSGGSGGVD-
DIQLTQSPAIMSASPGEKVTMTCRASSSVSYMNWYQQKSGTSPKRWIYDTS
KVASGVPYRESGSGSGTSYSLTISSMEAEDAATYYCQQWSSNPLTFGAGTK
LELK-GGGSGGGSGGGS-
CGQIEYLAKQIVDNAIQQAGC-GS-HHHHHH

### Example 4—Construction of Bispecific Antibodies Through Intracellular or Extracellular Assembling of IgG and scFv

Unlike previous design of trivalent DNL complexes that combined an anti-CD3 scFv with an anti-TAA F(ab)2 (U.S. Pat. No. 9,315,567 B2), the present invention relates to compositions and methods of novel constructs that link monovalent scFv to a full-size IgG through intracellular or extracellular biological conjugation. An exemplary set of these constructs are disclosed in this section, including the combination of anti-CD3 scFv (3scFv) with hL0125, a humanized full-size IgG specifically targeting Trop2. Other bispecific antibodies combining anti-CD3 or other scFv or protein with full-size IgGs against other tumor-associated antigens, discussed in more detail below.

Construct A: 3scFv×hL0125A

In this construct, a dimerization/docking domain (DDD2) from RIIα regulatory subunit of protein kinase A was added to the C-terminus of hL0125 light chain (LC) via a GS peptide linker. The sequences of the GS-DDD2 and LC-GS-DDD2 were shown below (SEQ ID NOs 16-17). The hL0125 IgG with LC-GS-DDD2 was designated as hL0125A.

GS-DDD2
(SEQ ID NO 16)
GGGGSGGGGSGGGGSGGGGS-
CGHIQIPPGLTELLQGYTVEVLRQQPPDIVEFAVEYFTRLREARA
LC-GS-DDD2
(SEQ ID NO 17)
DIQLTQSPAIMSASPGERVTMTCRASSSVSSSYLHWYQQRSGQSPKLLIYSTSNLASGVPAR
FSGSGSGTDYSLTISSLEAEDAATYYCQQYSGSPLTFGSGTKLEIKRTVAAPSVFIFPPSDE
QLKSGTASVVCLLNNFYPREAKVQWKVDNALQSGNSQESVTEQDSKDSTYSISSTLTLSKAD
YEKHKVYACEVTHQGLSSPVTKSFNRGEC-GGGGSGGGGSGGGGSGGGGS-
CGHIQIPPGLTELLQGYTVEVLRQQPPDIVEFAVEYFTRLREARA

A cDNA sequence encoding the GS-DDD2 (SEQ ID NO 16) was synthesized and added to the 3′ end of the hL0125 LC coding region to produce the IgG expression vector IgG V-hL0125A. The expression vector was transfected into the master cell line 3scFv-C21 expressing anti-CD3 single chain-AD2. Clones were selected in media containing 0.2 μM methotrexate (MTX) and 10 μg/ml puromycin and screened for human IgG expression. The anti-CD3 AD2 module 3scFv was intracellularly conjugated to hL0125A to produce the bispecific antibody 3scFv×hL0125A. The clone with high yield was scaled up to 500 ml culture and the bispecific antibody was purified from the broth by affinity chromatography using MabSelect™ resin, then HisPur Ni-NTA Resin, and analyzed by SDS-PAGE (FIG. 4).

Construct B: 3scFv×hL0125B

In this construct, a dimerization/docking domain (DDD2) from RIIα regulatory subunit of protein kinase A was added to the C-terminus of IgG LC of hL0125 via a PKSC-GS peptide linker. The sequences of the PKSC-GS-DDD2 and LC-PKSC-GS-DDD2 were shown below (SEQ ID NOs 18-19). The hL0125 IgG with LC-PKSC-GS-DDD2 was designated as hL0125B.

PKSC-GS-DDD2
(SEQ ID NO 18)
PKSC-GGGGSGGGGSGGG-
CGHIQIPPGLTELLQGYTVEVLRQQPPDIVEFAVEYFTRLREARA
LC-PKSC-GS-DDD2
(SEQ ID NO 19)
DIQLTQSPAIMSASPGERVTMTCRASSSVSSSYLHWYQQRSGQSPKLLIYSTSNLASGVPAR
FSGSGSGTDYSLTISSLEAEDAATYYCQQYSGSPLTFGSGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC-PKSC-GGGGSGGGGSGGG-
CGHIQIPPGLTELLQGYTVEVLRQQPPDLVEFAVEYFTRLREARA

A cDNA sequence encoding the PKSC-GS-DDD2 (SEQ ID NO 18) was synthesized and added to the 3′ end of the hL0125 LC coding region to produce the IgG expression vector IgG V-hL0125B. The expression vector was transfected into the master cell line 3scFv-C21 expressing anti-CD3 single chain-AD2. Clones were selected in media containing 02 μM methotrexate (MTX) and 10 μg/ml puromycin and screened for human IgG expression. The anti-CD3 AD2 module 3scFv was intracellularly conjugated with hL0125B to produce the bispecific antibody 3scFv×hL0125B. The clone with high yield was scaled up to 500 ml culture and the bispecific antibody was purified from the broth by affinity chromatography using MabSelect™ resin. Alternatively, the IgG V-hL0125B vector was transfected into different host cells. The 3scFv and hL0125B modules were purified from the broth of roller bottle cultures by affinity chromatography using HisPur™ Ni-NTA and MabSelect™ resins, respectively. The DNL™ method (Rossi et al, Proc Natl Acad Sci USA. 2006, 103(18):6841-6) was used to assemble 3scFv×hL0125B bispecific antibody via the in vitro conjugation of mole equivalents of 3scFV and hL0125B.

Construct C: 3scFv×hL0125C

In this construct, a dimerization/docking domain (DDD2) from RIIα regulatory subunit of protein kinase A was inserted into the hinge region of hL0125 IgG heavy chain (HC) via two GS peptide linkers. The sequences of GS-DDD2-GS and modified HIC of V_{H}-C_{H1}-hinge-GS-DDD2-GS-hinge-C_{H2}-C_{H3} were shown below (SEQ ID NOs 20-21). The hL0125 IgG with V_{H}-C_{H1}-hinge-GS-DDD2-GS-hinge-C_{H2}-C_{H3} was designated as hL0125C.

(SEQ ID NO 20)
GS-DDD2-GS
GGGGSGGGGSGGG-CGHIQIPPGLTELLQGYTVEVLRQQPPDLVEFAVEYE
TRLREARA-GGGGSGGGGSGGG
(SEQ ID NO 21)

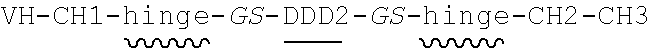

QVQLQESGGGLVKPGGSIKLSCAASGFTFSSYAMSWVRQTPDKRLEWVAEI
SSDGFYTYYPDTVTGRFTISRDNAKNTLYLOMSSLRSEDTAMYYCARDGNY
VDYAMDYWGQGTSVTVSS-ASTKGPSVFPLAPSSKSTSGGTAALGCLVKDY
FPEPVTVSWNSGALTSGVHTFPAVLQSSGLYSLDDBBTVPSSLGTQTYICN

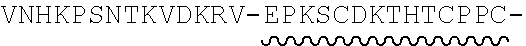

GGGGGGGGSGGG-CGHIQIPPGLTELLQGYTVEVLRQQPPDLVEFAVEYFT
RLREARA-

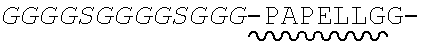

PSVFLFPPKPKDTIMISRTPEVTCVVVDVSHEDPEVKENWYVDGVEVHNA
KTKPREEQYNSTYRVVSVLTVLHQDWINGKEYKCKVSNKALPAPIEKTIS
KAK-
GQPREPQVYTLPPSREEMTKNQVSLTCLVKGFYPSDIAVEWESNGQPENN
YKTTPPVLDSDGSFFLYSKLTVDKSRWQQGNVESCSVMHEALHNHYTQKS
LSLSPGK

A cDNA sequence encoding the GS-DDD2-GS (SEQ ID NO 20) was synthesized and inserted into the hinge region of IgG HC of hL0125 to produce the IgG expression vector IgG V-hL0125C. The expression vector was transfected into the master cell line 3scFv-C21 expressing anti-CD3 single chain-AD2. Clones were selected in media containing 0.2 μM methotrexate (MTX) and 10 μg/ml puromycin and screened for human IgG expression. The anti-CD3 AD2 module 3scFv was intracellularly conjugated with hL0125C to produce the bispecific antibody 3scFv×hL0125C. The clone with high yield was scaled up to 500 ml culture and the bispecific antibody was purified from the broth by affinity chromatography using MabSelect™ resin and analyzed by SDS-PAGE.

Construct D: 3scFv×hL0125D

In this construct, a dimerization/docking domain (DDD2) from RIIα regulatory subunit of protein kinase A was added to the C-terminus of hL0125 IgG HIC via a (S peptide linker. The sequences of GS-DDD2 and modified HC of V_{H}-C_{H1}-hinge-CH2-CH3-GS-DDD2 were shown below (SEQ ID NOs 22-23). The hL0125 IgG with V_{H}-C_{H1}-hinge-C_{H2}-C_{H3}-GS-DDD2 was designated as hL0125D.

(SEQ ID NO 22)
GS-DDD2
GGGGSGGGGSGGG-CGHIQIPPGLTELLQGYTVEVLRQQPPDIVEFAVEYF
TRLREARA
(SEQ ID NO 23)

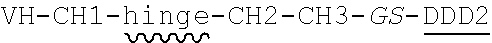

QVQLQESGGGLVKPGGSLKLSCAASGFTFSSYAMSWVRQTPDKRLEWVAEI
SSDGFYTYYPDTVTGRFTISRDNAKNTLYLQMSSLRSEDTAMYYCARDGNY
VDYAMDYWGQGTSVTVSS-
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV-

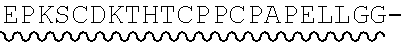

PSVFLFPPKPKDTIMISRTPEVTCVVVDVSHEDPEVKENWYVDGVEVHNAK
TKPREEQYNSTYRVVSVLTVLHQDWLNGKEYKCKVSNKALPAPIEKTISKA
K-
GQPREPQVYTIPPSREEMTKNQVSLTCLVKGFYPSDIAVEWESNGQPENNY
KTTPPVLDSDGSFFLYSKLTVDKSRWQQGNVFSCSVMHEALHNHYTQKSLS
LSPGK-
GGGGSGGGGSGGGCGHIQIPPGLTELLQGYTVEVLRQQPPDLVEFAVEYET
RLREARA

A cDNA sequence encoding the GS-DDD2 (SEQ ID NO 22) was synthesized and fused to the 3′ end of the hL0125 HC coding region to produce the IgG expression vector IgG V-hL0125D. The expression vector was transfected into the master cell line 3scFv-C21 expressing anti-CD3 single chain-AD2. Clones were selected in media containing 0.2 μM methotrexate (MTX) and 10 μg/ml puromycin and screened for human IgG expression. The anti-CD3 AD2 module 3scFv was intracellularly conjugated with hL0125D to produce the bispecific antibody 3scFv×hL0125D. The clone with high yield was scaled up to 500 ml culture and the bispecific antibody was purified from the broth by affinity chromatography using MabSelect™ resin and analyzed by SDS-PAGE.

Based on above constructs, four formats of bispecific antibodies, 3scFv×hL0125A, 3scFv×hL0125B, 3scFv×hL0125C, and 3scFv×hL0125D, were produced from four individual cell lines and purified from their supernatants by protein A selection. For comparison, their IgG-DDD2 modules hL0125A, hL0125B, hL0125C, and hL0125D were also produced in 4 different cell lines and purified from their supernatants by protein A selection. The anti-CD3 AD2 module 3scFv was produced in the master cell line 3scFv-C21 and purified by HisPur™ Ni-NTA Resin.

### SDS-PAGE Analyses

The anti-CD3 AD2 module 3scFv was well resolved with good purity under reducing condition but displayed as both monomer and dimer under non-reducing condition. All Ig-DDD2 modules, either expressed in separate cell lines or co-expressed with 3scFv in the master cell line 3scFv-C21, were well resolved under reducing condition but under non-reducing condition, existed as both monomer and dimer in hL0125A and hL0125D. In comparison, minimal dimers were observed in modules hL0125B and hL0125C under non-reducing condition though some degradation in hL0125B. When co-expressed in single cell, the anti-CD3 AD2 module and IgG-DDD2 module were intracellularly assembled to form four formats of 3scFv×hL0125X bispecific antibodies. The bispecific complex of 3scFv×hL0125A was partially formed in cells and unconjugated hL0125A with degradation was seen in the eluted product from Protein A column, which was removed by HisPur column (FIG. 4A). The bispecific complex of 3scFv×hL0125B showed good homogeneity when purified from low-yield clones (FIG. 4B), but aggregates formed in high-yield clones. This format of bispecific antibodies could be extracellularly assembled with better homogeneity via the site-specific DNL conjugation of mole equivalents of 3scFv and hL0125B (data not shown). The bispecific complexes of 3scFv×hL0125C and 3scFv×hL01251) were well generated in cells, showing good purity and yield after Protein A purification (FIGS. 4C and D). Considering the importance of proximity of two binding sites to form a tight immune synapse, the format of 3scFv×hL0125C was chosen for further study.

### Example 5—Cell Surface Binding of Bispecific Antibodies Targeting Both CD3 and Trop2

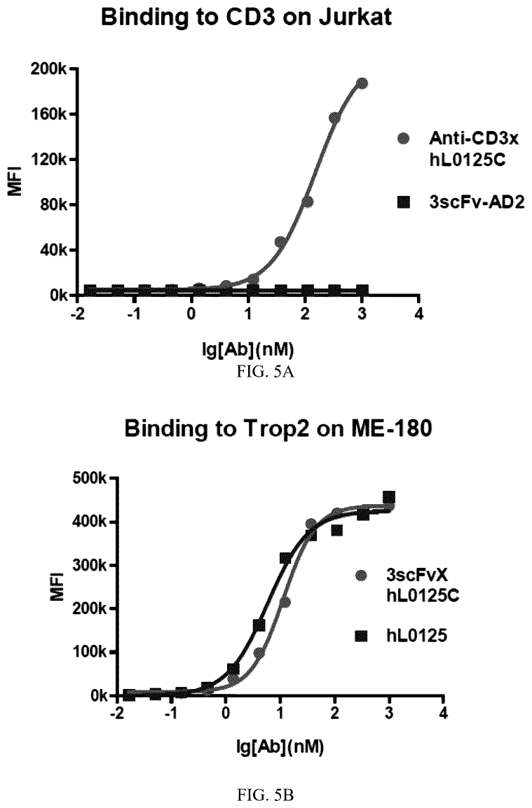

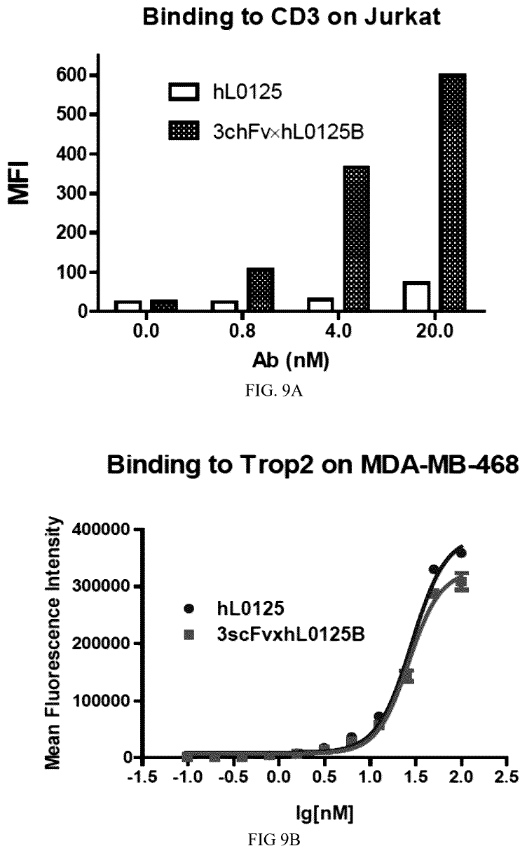

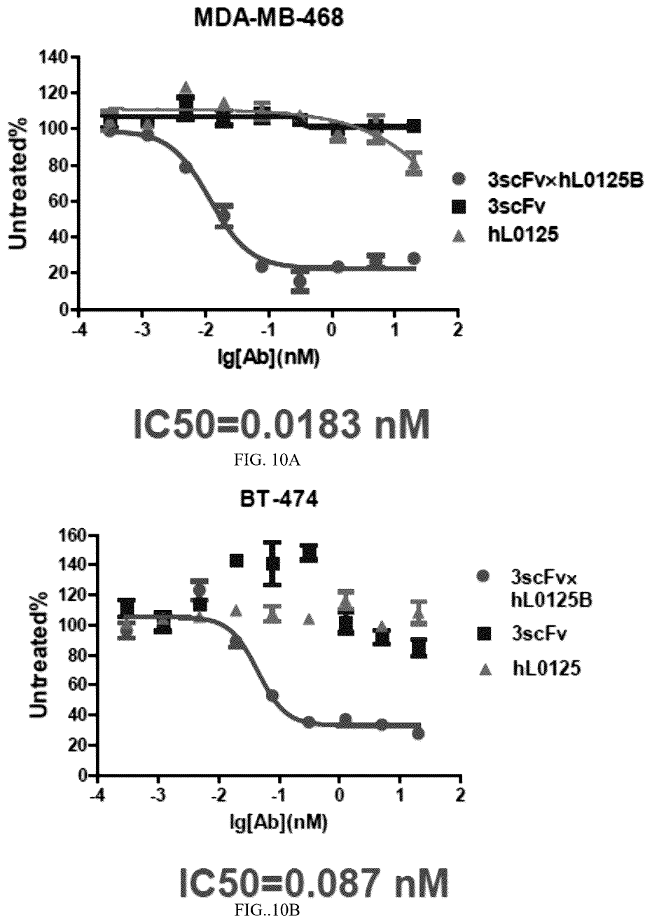

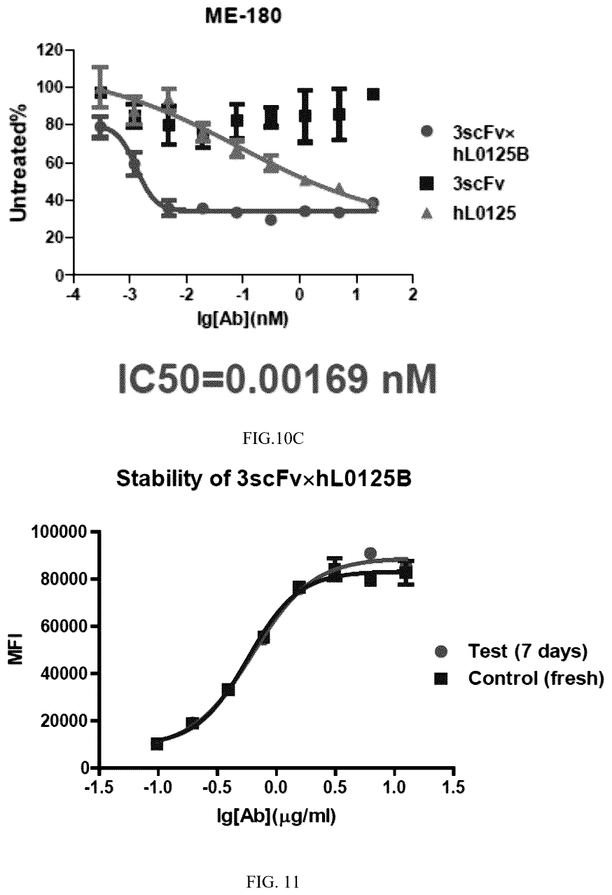

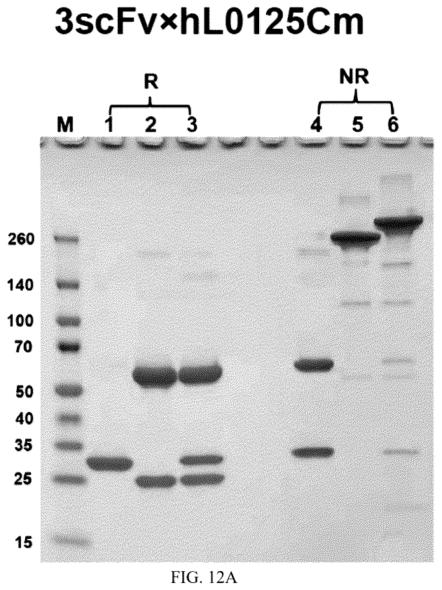

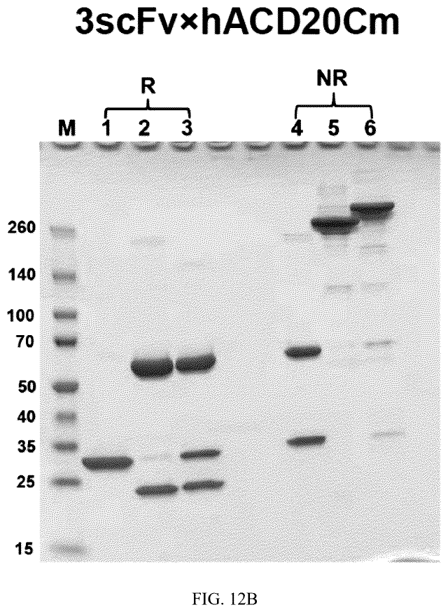

Flow cytometry was used to evaluate relative cell-binding strengths. For CD3 binding, Jurkat cells were dispensed into a 96-well plate at 2×10⁵/well, and incubated with 3-fold serially diluted bispecific antibodies (3scFv×hL0125C or 3scFv×hL0125B) or their modules at 4° C. for 45 min. After wash with PBS, cells were incubated with a AF488 labeled goat anti-human IgG Fc secondary antibody at 4° C. for another 45 min. After two wash steps, the cells were resuspended in PBS and analyzed using Attune N×T Flow Cytometer. The results confirmed that both bispecific antibodies 3scFv×hL0125C and 3scFv×hL0125B bind to human CD3 on Jurkat cell-surface, which were detected by a AF488 labeled goat anti-human IgG Fc secondary antibody (FIGS. 5 and 9). In comparison, no signal was detected for either 3scFv due to the lack of IgG Fc (FIG. 5) or hL0125 due to the lack of anti-CD3 domain (FIG. 9).

For Trop2 binding, ME-180 or MDA-MB-468 cells were dissociated from culture, dispensed into a 96-well plate at 2×10⁵/well, and incubated with 3-fold serially diluted bispecific antibodies (3scFv×hL0125C or 3scFv×hL0125B) or their modular antibody hL0125 at 4° C. for 45 min. After wash with PBS, cells were incubated with a AF488 labeled goat anti-human IgG Fc secondary antibody at 4° C. for another 45 min. After two wash steps, binding was analyzed by flow cytometry using Attune N×T Flow Cytometer. The results demonstrated that the bispecific antibody of either 3scFv×hL0125C or 3scFv×hL0125B binds to human Trop2 on ME-180 or MDA-MB-468 with similar affinity to the modular antibody hL0125.

### Example 6—Cytotoxicity Assay

### Cytotoxicity Assays for Tumor Cells Grown in Monolayer.

Cancer cells were combined with human PBMCs and dispensed into 96-well plates at 200 μl/well to provide 1×10⁴ tumor cells and 1×10⁵ PBMC cells (PBMC-to-target ratio of 10:1) in each well. Bispecific antibodies (3scFv×hL0125C or 3scFv×hL0125B) or their monospecific modules starting at 20 nmol/L, were 4-fold serially diluted to treat the mixed cells. After 60 h incubation, media were removed and replaced with fresh media to flush PBMC cells and dead cancer cells twice. Cell viabilities were measured with MTS reagent. For 3scFv×hL0125C, the assay was done in triplicates with breast cancer lines MDA-MB-468, MDA-MB-231, BT-474, and colon cancer cell line HCT116. For 3scFv×hL0125B, the assays were done in triplicates with breast cancer lines MDA-MB-468, BT-474, and cervical cancer cell line ME-180.

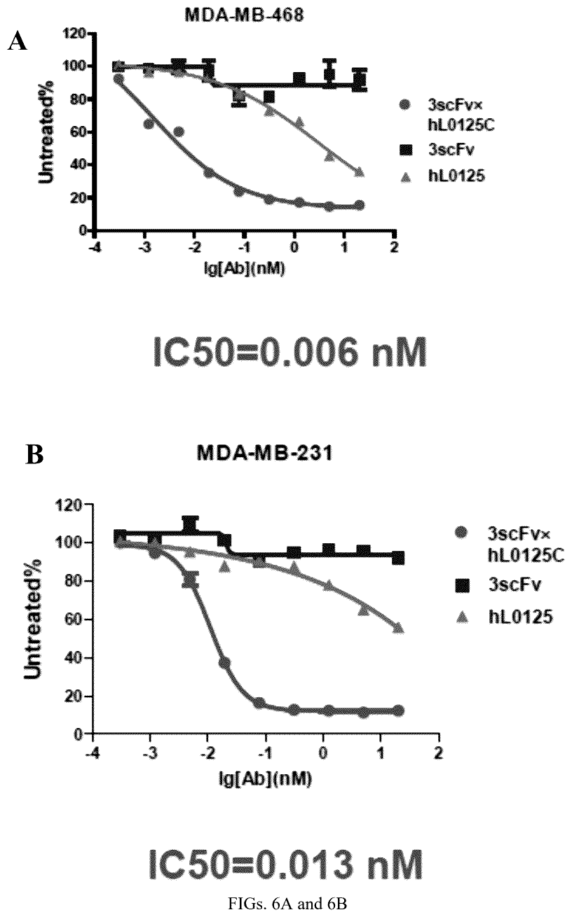

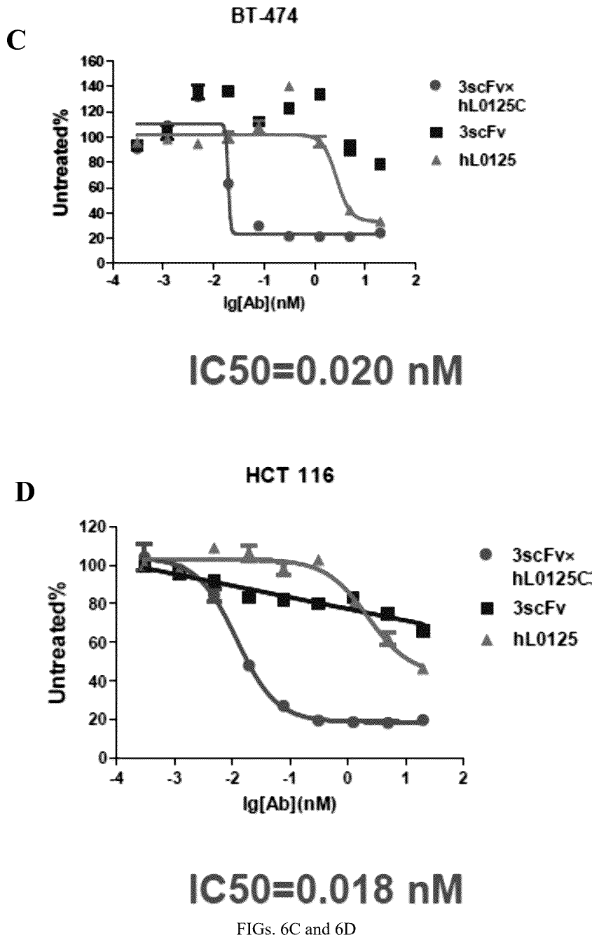

Bispecific antibodies mediated dose-dependent killing of tumor cells derived from breast (MDA-MB-468, MDA-MB-231, BT-474), colorectal (HCT 116), and cervical (ME-180) cancer cells. Based on the 60-hour viability assays using MTS, the IC₅₀ of 3scFv×hL0125C was about 0.006 nM for MDA-MB-468, 0.013 nM for MDA-MB-231, 0.02 nM for BT-474, and 0.018 nM for HCT116 (FIG. 6). The IC₅₀ of 3scFv×hL0125B was about 0.0183 nM for MDA-MB-231, 0.087 nM for BT-474, and 0.00169 nM for ME-180 (FIG. 10). The extraordinary potency of bispecific antibody-induced cytotoxicity was demonstrated by comparing redirected killing of Trop2-expressing cells with that of its modules 3scFv and hL0125. Minimal or little target cell killing was observed with either 3scFv or hL0125 under identical condition (FIGS. 6 and 10)

### Formation of Multicellular Tumor Spheroids or Cytotoxicity Evaluation.

HCT 116 cells were combined with human PBMCs and dispensed into 96-well spheroid microplates at 200 μl/well to provide 200 tumor cells and 1,600 PBMCs (PBMC-to-target ratio of 8:1) in each well. Bispecific Ab (3scFv×hL0125C) and its monospecific modules starting at 20 nmol/L, were 4-fold serially diluted to treat the mixed cells, After 7-day incubation, tumor in each well was captured using EVOS FL Auto 2. The assay was done in duplicates.

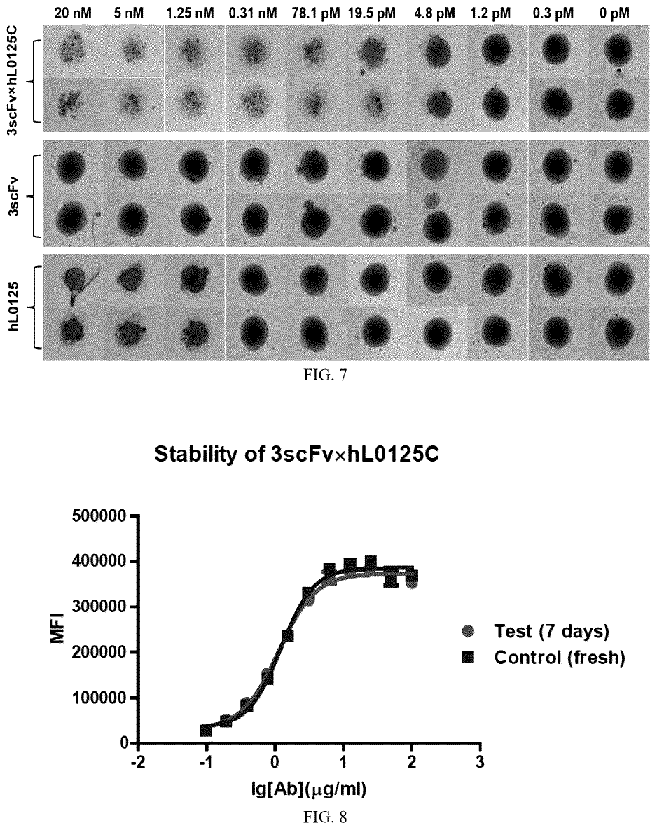

As shown in FIG. 7, the formation of multicellular tumor spheroids was apparently impeded by 3scFv×hL0125C at the concentrations of 19.5 pM or above, which is consistent with the cytotoxicity assays for tumor cells grown in monolayer, little or minimal effects were observed at cultures treated with either 3scFv or hL0125. The results confirmed the cytotoxicity of bispecific complex of 3scFv×hL0125C.

### Example 7—Stability Analysis of Bispecific Antibodies in Human Plasma

Bispecific samples were diluted from 1.63 mg/A (3scFv×hL0125C) or 1.25 mg/ml (3scFv×hL0125B) to 100 μg/ml human plasm and incubated at 37° C. for 7 days. Afterwards, the stability was checked by cell surface binding and detection. Specifically, sample was 2-fold diluted in PBS and incubated with MDA-MB-468 cells for binding to cell surface Trop2 at 4° C. for 45 min, then detected by anti-His-AF488 (the anti-CD3-AD2 module was His-tagged at C-terminus). After two wash steps, binding was analyzed by flow cytometry using Attune N×T Flow Cytometer. The control sample was diluted to 100 μg/ml in human plasm and immediately analyzed for similar binding assay.

The assay was designed to examine the intactness and cell surface binding activity of the two bispecific antibodies of 3scFv×hL0125C and 3scFv×hL0125B treated in human plasm for 7 days at 37° C. Plasm-treated samples were compared with control samples that were freshly prepared from stock solutions. Both 3scFv×hL0125C and 3scFv×hL0125B are stable in plasm, retaining bispecific intactness and binding activity after 7 days (FIGS. 8 and 11).

### Example 8—Production of Bispecific 3scFv×XCm with Ablated FcγR Binding

3scFv×hL0125Cm

A critical component of Fc-containing T cell redirecting bispecific antibodies is the ablation of Fc γ R binding from the Fc region, thus eliminating off-target T cell activation by Fc γ R-expressing cells and the potential for T cell lysis mediated by Fc γ R-expressing effector cells. Based on previous findings (Moore et al, Methods. 2019, 154:38-50), FcγRs interact with antibodies primarily by contacting the hinge and CH2 domains, and IgG2 antibodies are known to have much weaker affinity for FcγRs than human IgG1. In this example, the hL125C module in the bispecific antibody 3scFv×hL0125C was modified to produce the IgG1 variant (hL0125Cm) that combined the Ig02 lower hinge substitution with an additional CH2 substitution at a surface residue (SEQ ID NO 24). The IgG expression vector IgG V-hL0125Cm was transfected into the master cell line expressing anti-CD3 single chain-AD2 (3scFv). Clones were selected in media containing 0.2 μM methotrexate (MTX) and 10 ug/nil puromycin and screened for human IgG expression. The anti-CD3 AD2 module 3scFv was intracellularly conjugated with hL0125Cm to produce the bispecific antibody 3scFv×hL0125Cm. The clone with high yield was scaled up to 500 ml culture and the bispecific antibody was purified from the broth by affinity chromatography using MabSelect™ resin and analyzed by SDS-PAGE.

(SEQ ID NO 24)

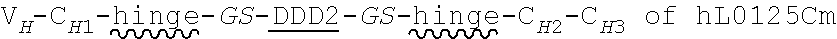

QVQLQESGGGLVKPGGSLKLSCAASGFTFSSYAMSWVRQTPDKRLEWVAEI
SSDGFYTYYPDTVTGRFTISRDNAKNTLYLQMSSLRSEDTAMYYCARDGNY
VDYAMDYWGQGTSVTVSS-
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV-

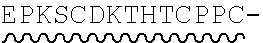

GGGGSGGGGSGGG-CGHIQIPPGLTELLQGYTVEVLRQQPPDLVEFAVEYF
TRLREARA-

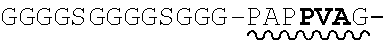

PSVFLFPPKPKDTLMISRTPEVTCVVVDVKHEDPEVKENWYVDGVEVHNAK
TKPREEQYNSTYRVVSVLTVLHQDWLNGKEYKCKVSNKALPAPIEKTISKA
K-
GQPREPQVYTIPPSREEMTKNQVSLTCLVKGFYPSDIAVEWESNGQPENNY
KTTPPVLDSDGSFFLYSKLTVDKSRWQQGNVFSCSVMHEALHNHYTQKSLS
LSPGK

The SDS-PAGE results show that the substitutions in the hinge and CH2 domains of hL0125Cm do not affect its conjugation with 3scFv. The bispecific complex of 3scFv×hL0125Cm has similar SDS-PAGE pattern when compared with 3scFv×hL0125C (FIGS. 12 and 4).

3scFv×hACD20Cm

The construction format of 3scFv×hL0125Cm was extended to other antibody moieties targeting various disease-associated antigens, combined with the anti-CD3 antibody moiety, to produce bispecific antibodies generally abbreviated as 3scFv×XCm. As an example, the V_{H} and V_{L} of hL0125Cm were substituted to V_{H} and V_{L} of veltuzumab, a humanized anti-CD20 monoclonal antibody, to produce the bispecific antibody 3scFv×hACD20Cm. Specifically, in the IgG expression vector of IgG V-hL0125Cm, the cDNA sequence encoding V_{H} of hL0125Cm was substituted to the cDNA sequence encoding V_{H} of veltuzumab, the cDNA sequence encoding V_{L} of hL0125Cm was substituted to the cDNA sequence encoding V_{L} of veltuzumab, resulting in the IgG vector of IgG V-hACD20Cm, which expressed the heavy and light chains of hACD20Cm (SEQ ID NOs 25-26). The IgG expression vector IgG V-hACD20Cm was transfected into the master cell line expressing anti-CD3 single chain-AD2 (3scFv). Clones were selected in media containing 0.2 μM methotrexate (MTX) and 10 μg/ml puromycin and screened for human IgG expression. The anti-CD3 AD2 module 3scFv was intracellularly conjugated with hACD20Cm to produce the bispecific antibody 3scFv×hACD20Cm. The clone with high yield was scaled up to 500 ml culture and the bispecific antibody was purified from the broth by affinity chromatography using MabSelect™ resin and analyzed by SDS-PAGE.

(SEQ ID NO 25)

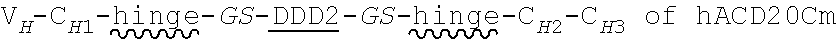

QVQLQQSGAEVKKPGSSVKVSCKASGYTFTSYNMHWVKQAPGQGLEWIGAI
YPGNGDTSYNQKFKGKATLTADESTNTAYMELSSLRSEDTAFYYCARSTYY
GGDWYFDVWGQGTTVTVSS-
ASTKGPSVFPLAPSSKSTSGGTAALGCLVKDYFPEPVTVSWNSGALTSGVH
TFPAVLQSSGLYSLSSVVTVPSSSIGTQTYICNVNHKPSNTKVDKRV-

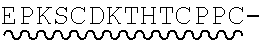

GGGGSGGGGSGGG-CGHIQIPPGLTELLQGYTVEVLRQQPPDIVEFAVEYF
TRLREARA-

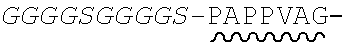

PSVFLFPPKPKDTLMISRTPEVTCVVVDVKHEDPEVKENWYVDGVEVHNAK
TKPREEQYNSTYRVVSVLTVLHQDWLNGKEYKCKVSNKALPAPIEKTISKA
K-
GQPREPQVYTIPPSREEMTKNQVSLTCLVKGFYPSDIAVEWESNGQPENNY
KTTPPVLDSDGSFFLYSKLTVDKSRWQQGNVESCSVMHEALHNHYTQKSLS
LSPGK
(SEQ ID NO 26)
V_{L}-C_{L} of hACD20Cm
DIQLTQSPSSLSASVGDRVTMTCRASSSVSYIHWFQQKPGKAPKPWIYATS
NLASGVPVRESGSGSGTDYTFTISSLQPEDIATYYCQQWTSNPPTFGGGTK
LEIKR-
TVAAPSVFIFPPSDEQLKSGTASVVCLLNNFYPREAKVOWKVDNALQSGNS
QESVTEQDSKDSTYSLSSTLILSKADYEKHKVYACEVTHQGLSSPVTKSEN
RGEC

As shown in FIG. 12, the bispecific antibody of 3scFv×hACD20Cm was successfully produced with comparable yield and quality to 3scFv×hL0125Cm. This result confirms that the bispecific format of 3scFv×hL0125Cm can be applied to other antibody moieties targeting various disease-associated antigens, in combination with the anti-CD3 antibody moiety or other therapeutic proteins, to produce bispecific molecules. Likewise, other bispecific formats, including 3scFv×hL0125A, 3scFv×hL0125B, 3scFv×hL0125C, and 3scFv×hL0125D, would be applied to other antibody moieties targeting various disease-associated antigens, in combination with the anti-CD3 antibody moiety or other therapeutic proteins, to produce bispecific molecules.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the disclosure, and all such variations are intended to be included within the scope of the following claims. All references cited herein are incorporated by reference in their entireties.

1. A protein complex, comprising
a first moiety comprising two immunoglobulin light chains and two immunoglobulin heavy chains, wherein either the two light chains or the two heavy chains are linked to two dimerization/docking domain (DDD) moieties respectively, and
a second moiety comprising (i) an anchoring domain (AD) moiety and (ii) an agent linked to the AD moiety, wherein the two DDD moieties form a dimer that binds to the AD moiety.

2. The protein complex of claim 1, wherein
(a) the first moiety is a targeting moiety that specifically binds to an antigen or epitope, and
(b) the second moiety is an effector moiety, and the agent is an effector agent.

3. The protein complex of claim 1, wherein
(a) the first moiety is an effector moiety comprising an effector agent and
(b) the second moiety is a targeting moiety and the agent is a targeting agent that specifically binds to an antigen or epitope.

4. The protein complex of claim 1, wherein
(a) the first moiety and the second moiety are two targeting moieties that specifically bind to two antigens or epitopes, or
(b) the first moiety and the second moiety are two effector moieties, and the agents are effector agents.

5. A fusion protein comprising
(i) a dimerization/docking domain (DDD) moiety and
(ii) an immunoglobulin light chain fused to the DDD moiety, or an immunoglobulin heavy chain fused to the DDD moiety.

6. The protein complex of claim 1, wherein each DDD moiety is inserted in each immunoglobulin heavy chain, or
is fused to the C-terminus of each immunoglobulin light chain, or
is fused to the C-terminus of each immunoglobulin heavy chain.

7-11. (canceled)

12. The protein complex of claim 2, wherein the targeting moiety binds specifically to a tumor associated antigen or a disease associated antigen.

13. The protein complex of claim 12, wherein the tumor associated antigen or the disease associated antigen is Trop2, EpCAM, GPRC5, FcRH5, ROR1, BCMA, CD15, CD16, CD19, CD20, CD22, CD27, CD30, CD33, CD40, CD47, CD40L, CD66, CD70, CD74, CD79b, CD80, CD95, CD133, CD160, CD166, CD229, MUC1, MUC5, MUC16, IGF-1R, EGFR, HER2, HER3, EGP2, HLA-DR, TNF-α, TRAIL receptor, ICOS, ICOSL, VEGF, VEGFR, hypoxia inducible factor (HIF), Flt-3, folate receptor, TDGF1, TfR, Mesothelin, PSMA, CEACAM5, CEACAM6, B7, IFN-α, IFN-0, IFN-7, IFN-k, IL-10, IL2, IL6, IL-6R, IL-15, IL-15R, IL-17, IL-17R, IL-12, C1r, C1s, C2, C3, C5, C5a, C5aR1, C6, MASPs, MSAP2, MASP3, FB, FD, Properdin, Lag-3, CTLA-4, PD-1, PD-L1, TIM3, SIRPα, TIGIT, OX40, OX40L, 4-1BB, NKG2A, NKG2B, BTLA, GITR, GITRL, TCR, Nectin-4, c-Met, LIV1, Mesothelin, DLL3, DLL4, Tissue factor, TGF-β, TGF-β receptor, DKK1, or CLDN18.2.

14. The protein complex of claim 1 wherein
the immunoglobulin light chain comprises a light chain variable region comprising LCDR1, LCDR2 and LCDR3, wherein the LCDR1, LCDR2 and LCDR3 comprise the respective sequences of SEQ ID NOs: 27-29 and/or
the immunoglobulin heavy chain comprises a heavy chain variable region comprising HCDR1, HCDR2 and HCDR3, wherein the HCDR1, HCDR2 and HCDR3 comprise the respective sequences of SEQ ID NOs: 30-32.

15. The protein complex of claim 14, wherein
the immunoglobulin light chain comprises the sequence of SEQ ID NO: 17 or 19, or
the immunoglobulin heavy chain comprises the sequence of SEQ ID NO: 21 or 23 or 24.

16. The protein complex of claim 1, wherein
the immunoglobulin light chain comprises the sequence of SEQ ID NO: 26, and/or
the immunoglobulin heavy chain comprises the sequence of SEQ ID NO: 25.

17. The protein complex of claim 2, wherein the effector agent comprises an antibody or an antigen-binding fragment thereof, aptamers, a ligand, a cytotoxin, a chemotherapeutic agent, a detectable label or tag, a drug, a pro-drug, a toxin, an enzyme, an immunomodulator, a checkpoint inhibitor, an anti-angiogenic agent, a pro-apoptotic agent, a cytokine, a growth factor, a hormone, a cytokine, a radioisotope, a protein, a peptide, a peptide mimetic, a polynucleotide, a RNAi oligosaccharide, a natural or synthetic polymeric substance, a nanoparticle, a quantum dot, an organic compound, or an inorganic compound.

18-20. (canceled)

21. The protein complex of claim 17, wherein the antibody comprises the sequence of 3scFv (SEQ ID NO: 33).

22. An antibody or antigen-binding fragment thereof that specifically binds to human Trop2, comprising:
(i) a light chain or a light chain variable region that comprises LCDR1, LCDR2 and LCDR3 comprising the respective sequences of SEQ ID NOs: 27-29, and/or
(ii) a heavy chain or a heavy chain variable region that comprises HCDR1, HCDR2, and HCDR3 comprising the respective sequences of SEQ ID NOs: 30-32.

23. The antibody or antigen-binding fragment thereof of claim 22, wherein the light chain variable region that comprises the sequence of SEQ ID NO: 1 or 5, and/or the heavy chain variable region that comprises the sequence of SEQ ID NO: 3 or 7.

24. (canceled)

25. A nucleic acid sequence or nucleic acid sequences encoding a protein complex of claim 1, or an expression vector composing the nucleic acid sequence or nucleic acid sequences.

26. (canceled)

27. A host cell comprising the nucleic acid or nucleic acids or the expression vector of claim 25.

28. A method of preparing a protein complex or fusion protein or antibody or antigen-binding fragment, comprising:
obtaining a cultured host cell comprising a nucleic acid sequence or nucleic acid sequences encoding the protein complex of claim 1,
culturing the cell in a medium under conditions permitting expression of protein complex and assembling of the protein complex inside the cell or outside the cells, and
purifying the protein complex from the cultured cell or the medium of the cell.

29. (canceled)

30. A pharmaceutical composition comprising the protein complex of claim 1 and a pharmaceutically acceptable carrier.

31. A method for treating a cancer or a disease in a subject in need thereof, comprising administering to the subject an effective amount of the protein complex of claim 1.


SEQUENCE LISTING (147 sequences)

SEQ ID NO: 1 - protein, 109 aa, synthetic construct
DIQLTQSPAIMSASPGEKVTMTCRASSSVSSSYLHWYQQRSGASPKLWIYSTSNLASGVPARFSGSGSGTSYSLTISSVEAEDAATYYCQQYSGSPLTFGSGTKLEIKR

SEQ ID NO: 2 - DNA, 327 bp, synthetic construct
  source 1..327: mol_type = other DNA
gacattcagctgacccagtctccagcaatcatgtctgcatctccaggggaaaaggtcaccatgacctgcagggccagctcaagtgtaagttccagttacttgcactggtaccagcagaggtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtggttccccactcacgttcggctcggggacaaagttggaaataaaacgg

SEQ ID NO: 3 - protein, 119 aa, synthetic construct
VKLQESGGGLVKPGGSLKLSCAASGFTFSSYAMSWVRQSPEKRLEWVAEISSDGFYTYYPDTVTGRFTISRDNAKNTLYLEMSSLRSEDTAMYYCARDGNYVDYAMDYWGQGTSVTVSS

SEQ ID NO: 4 - DNA, 357 bp, synthetic construct
  source 1..357: mol_type = other DNA
gtgaaactgcaggagtcagggggaggcttagtgaagcctggagggtccctgaaactctcctgtgcagcctctggattcactttcagtagctatgccatgtcttgggttcgccagtctccagagaagaggctggagtgggtcgccgaaattagtagtgatggtttttacacctactatccagacactgtgacgggccgattcaccatctccagagacaatgccaagaacaccctgtacctggaaatgagcagtctgaggtctgaggacacggccatgtattactgtgcaagggatggtaactacgtggactatgctatggactactggggtcaaggaacctcagtcaccgtctcctca

SEQ ID NO: 5 - protein, 109 aa, synthetic construct
DIQLTQSPAIMSASPGERVTMTCRASSSVSSSYLHWYQQRSGQSPKLLIYSTSNLASGVPARFSGSGSGTDYSLTISSLEAEDAATYYCQQYSGSPLTFGSGTKLEIKR

SEQ ID NO: 6 - DNA, 327 bp, synthetic construct
  source 1..327: mol_type = other DNA
gacattcagctgacccagtctccagcaatcatgtctgcatctccaggggaaagggtcaccatgacctgcagggccagctcaagtgtaagttccagttacttgcactggtaccagcagaggtcaggtcagtcccccaaactcctgatttatagcacatccaacttggcttctggagtccctgctcgcttcagtggcagtgggtctgggaccgattactctctcacaatcagcagtctcgaggctgaagatgctgccacttattactgccagcagtacagtggttccccactcacgttcggctcggggacaaagttggaaataaaacgg

SEQ ID NO: 7 - protein, 120 aa, synthetic construct
QVQLQESGGGLVKPGGSLKLSCAASGFTFSSYAMSWVRQTPDKRLEWVAEISSDGFYTYYPDTVTGRFTISRDNAKNTLYLQMSSLRSEDTAMYYCARDGNYVDYAMDYWGQGTSVTVSS

SEQ ID NO: 8 - DNA, 360 bp, synthetic construct
  source 1..360: mol_type = other DNA
caggtgcagctgcaggagtcagggggaggcttagtgaagcctggagggtccctgaaactctcctgtgcagcctctggattcactttcagtagctatgccatgtcttgggttcgccagacaccagacaagaggctggagtgggtcgccgaaattagtagtgatggtttttacacctactatccagacactgtgacgggccgattcaccatctccagagacaatgccaagaacaccctgtacctgcaaatgagcagtctgaggtctgaggacacggccatgtattactgtgcaagggatggtaactacgtggactatgctatggactactggggtcaaggaacctcagtcaccgtctcctca

SEQ ID NO: 9 - protein, 19 aa, synthetic construct
MGWSCIILFLVATATGVHS

SEQ ID NO: 10 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 11 - protein, 18 aa, synthetic construct
VEGGSGGSGGSGGSGGVD

SEQ ID NO: 12 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 13 - protein, 12 aa, synthetic construct
GGGSGGGSGGGS

SEQ ID NO: 14 - protein, 21 aa, synthetic construct
CGQIEYLAKQIVDNAIQQAGC

SEQ ID NO: 15 - protein, 8 aa, synthetic construct
GSHHHHHH

SEQ ID NO: 16 - protein, 65 aa, synthetic construct
GGGGSGGGGSGGGGSGGGGSCGHIQIPPGLTELLQGYTVEVLRQQPPDLVEFAVEYFTRLREARA

SEQ ID NO: 17 - protein, 280 aa, synthetic construct
DIQLTQSPAIMSASPGERVTMTCRASSSVSSSYLHWYQQRSGQSPKLLIYSTSNLASGVPARFSGSGSGTDYSLTISSLEAEDAATYYCQQYSGSPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSCGHIQIPPGLTELLQGYTVEVLRQQPPDLVEFAVEYFTRLREARA

SEQ ID NO: 18 - protein, 62 aa, synthetic construct
PKSCGGGGSGGGGSGGGCGHIQIPPGLTELLQGYTVEVLRQQPPDLVEFAVEYFTRLREARA

SEQ ID NO: 19 - protein, 277 aa, synthetic construct
DIQLTQSPAIMSASPGERVTMTCRASSSVSSSYLHWYQQRSGQSPKLLIYSTSNLASGVPARFSGSGSGTDYSLTISSLEAEDAATYYCQQYSGSPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPKSCGGGGSGGGGSGGGCGHIQIPPGLTELLQGYTVEVLRQQPPDLVEFAVEYFTRLREARA

SEQ ID NO: 20 - protein, 71 aa, synthetic construct
GGGGSGGGGSGGGCGHIQIPPGLTELLQGYTVEVLRQQPPDLVEFAVEYFTRLREARAGGGGSGGGGSGGG

SEQ ID NO: 21 - protein, 521 aa, synthetic construct
QVQLQESGGGLVKPGGSLKLSCAASGFTFSSYAMSWVRQTPDKRLEWVAEISSDGFYTYYPDTVTGRFTISRDNAKNTLYLQMSSLRSEDTAMYYCARDGNYVDYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCGGGGSGGGGSGGGCGHIQIPPGLTELLQGYTVEVLRQQPPDLVEFAVEYFTRLREARAGGGGSGGGGSGGGPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 58 aa, synthetic construct
GGGGSGGGGSGGGCGHIQIPPGLTELLQGYTVEVLRQQPPDLVEFAVEYFTRLREARA

SEQ ID NO: 23 - protein, 508 aa, synthetic construct
QVQLQESGGGLVKPGGSLKLSCAASGFTFSSYAMSWVRQTPDKRLEWVAEISSDGFYTYYPDTVTGRFTISRDNAKNTLYLQMSSLRSEDTAMYYCARDGNYVDYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGCGHIQIPPGLTELLQGYTVEVLRQQPPDLVEFAVEYFTRLREARA

SEQ ID NO: 24 - protein, 520 aa, synthetic construct
QVQLQESGGGLVKPGGSLKLSCAASGFTFSSYAMSWVRQTPDKRLEWVAEISSDGFYTYYPDTVTGRFTISRDNAKNTLYLQMSSLRSEDTAMYYCARDGNYVDYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCGGGGSGGGGSGGGCGHIQIPPGLTELLQGYTVEVLRQQPPDLVEFAVEYFTRLREARAGGGGSGGGGSGGGPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 521 aa, synthetic construct
QVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCGGGGSGGGGSGGGCGHIQIPPGLTELLQGYTVEVLRQQPPDLVEFAVEYFTRLREARAGGGGSGGGGSGGGPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 213 aa, synthetic construct
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 12 aa, synthetic construct
RASSSVSSSYLH

SEQ ID NO: 28 - protein, 7 aa, synthetic construct
STSNLAS

SEQ ID NO: 29 - protein, 9 aa, synthetic construct
QQYSGSPLT

SEQ ID NO: 30 - protein, 10 aa, synthetic construct
GFTFSSYAMS

SEQ ID NO: 31 - protein, 17 aa, synthetic construct
EISSDGFYTYYPDTVTG

SEQ ID NO: 32 - protein, 11 aa, synthetic construct
DGNYVDYAMDY

SEQ ID NO: 33 - protein, 284 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGSGGGSGGGSCGQIEYLAKQIVDNAIQQAGCGSHHHHHH

SEQ ID NO: 34 - protein, 5 aa, synthetic construct
VDKRV

SEQ ID NO: 35 - protein, 5 aa, synthetic construct
VDKTV

SEQ ID NO: 36 - protein, 10 aa, synthetic construct
EPKSCDKTHT

SEQ ID NO: 37 - skipped

SEQ ID NO: 38 - protein, 12 aa, synthetic construct
ELKTPLGDTTHT

SEQ ID NO: 39 - protein, 4 aa, synthetic construct
EPKS

SEQ ID NO: 40 - protein, 7 aa, synthetic construct
ESKYGPP

SEQ ID NO: 41 - protein, 5 aa, synthetic construct
CPPCP

SEQ ID NO: 42 - protein, 9 aa, synthetic construct
CCVECPPCP

SEQ ID NO: 43 - protein, 5 aa, synthetic construct
CPRCP

SEQ ID NO: 44 - protein, 15 aa, synthetic construct
EPKSCDTPPPCPRCP

SEQ ID NO: 45 - protein, 11 aa, synthetic construct
CDTPPPCPRCP

SEQ ID NO: 46 - protein, 5 aa, synthetic construct
CPSCP

SEQ ID NO: 47 - protein, 7 aa, synthetic construct
APELLGG

SEQ ID NO: 48 - protein, 6 aa, synthetic construct
APPVAG

SEQ ID NO: 49 - protein, 330 aa, synthetic construct [sequence omitted]

SEQ ID NO: 50 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 51 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 52 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 54 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 55 - protein, 9 aa, synthetic construct
GCCGCCACC

SEQ ID NO: 56 - protein, 44 aa, synthetic construct
SHIQIPPGLTELLQGYTVEVLRQQPPDLVEFAVEYFTRLREARA

SEQ ID NO: 57 - protein, 45 aa, synthetic construct
CGHIQIPPGLTELLQGYTVEVLRQQPPDLVEFAVEYFTRLREARA

SEQ ID NO: 58 - protein, 50 aa, synthetic construct
SLRECELYVQKHNIQALLKDSIVQLCTARPERPMAFLREYFERLEKEEAK

SEQ ID NO: 59 - protein, 44 aa, synthetic construct
SIEIPAGLTELLQGFTVEVLRHQPADLLEFALQHFTRLQQENER

SEQ ID NO: 60 - protein, 54 aa, synthetic construct
SLKGCELYVQLHGIQQVLKDCIVHLCISKPERPMKFLREHFEKLEKEENRQILA

SEQ ID NO: 61 - protein, 17 aa, synthetic construct
QIEYLAKQIVDNAIQQA

SEQ ID NO: 62 - protein, 23 aa, synthetic construct
CGFEELAWKIAKMIWSDVFQQGC

SEQ ID NO: 63 - protein, 44 aa, synthetic construct
THIQIPPGLTELLQGYTVEVLRQQPPDLVEFAVEYFTRLREARA

SEQ ID NO: 64 - protein, 44 aa, synthetic construct
SKIQIPPGLTELLQGYTVEVLRQQPPDLVEFAVEYFTRLREARA

SEQ ID NO: 65 - protein, 44 aa, synthetic construct
SRIQIPPGLTELLQGYTVEVLRQQPPDLVEFAVEYFTRLREARA

SEQ ID NO: 66 - protein, 44 aa, synthetic construct
SHINIPPGLTELLQGYTVEVLRQQPPDLVEFAVEYFTRLREARA

SEQ ID NO: 67 - protein, 44 aa, synthetic construct
SHIQIPPALTELLQGYTVEVLRQQPPDLVEFAVEYFTRLREARA

SEQ ID NO: 68 - protein, 44 aa, synthetic construct
SHIQIPPGLSELLQGYTVEVLRQQPPDLVEFAVEYFTRLREARA

SEQ ID NO: 69 - protein, 44 aa, synthetic construct
SHIQIPPGLTDLLQGYTVEVLRQQPPDLVEFAVEYFTRLREARA

SEQ ID NO: 70 - protein, 44 aa, synthetic construct
SHIQIPPGLTELLNGYTVEVLRQQPPDLVEFAVEYFTRLREARA

SEQ ID NO: 71 - protein, 44 aa, synthetic construct
SHIQIPPGLTELLQAYTVEVLRQQPPDLVEFAVEYFTRLREARA

SEQ ID NO: 72 - protein, 44 aa, synthetic construct
SHIQIPPGLTELLQGYSVEVLRQQPPDLVEFAVEYFIRLREARA

SEQ ID NO: 73 - protein, 44 aa, synthetic construct
SHIQIPPGLTELLQGYTVDVLRQQPPDLVEFAVEYFTRLREARA

SEQ ID NO: 74 - protein, 44 aa, synthetic construct
SHIQIPPGLTELLQGYTVEVLKQQPPDLVEFAVEYFTRLREARA

SEQ ID NO: 75 - protein, 44 aa, synthetic construct
SHIQIPPGLTELLQGYTVEVLRNQPPDLVEFAVEYFTRLREARA

SEQ ID NO: 76 - protein, 44 aa, synthetic construct
SHIQIPPGLTELLQGYTVEVLRQNPPDLVEFAVEYFTRLREARA

SEQ ID NO: 77 - protein, 44 aa, synthetic construct
SHIQIPPGLTELLQGYTVEVLRQQPPELVEFAVEYFTRLREARA

SEQ ID NO: 78 - protein, 44 aa, synthetic construct
SHIQIPPGLTELLQGYTVEVLRQQPPDLVDFAVEYFTRLREARA

SEQ ID NO: 79 - protein, 44 aa, synthetic construct
SHIQIPPGLTELLQGYTVEVLRQQPPDLVEFLVEYFTRLREARA

SEQ ID NO: 80 - protein, 44 aa, synthetic construct
SHIQIPPGLTELLQGYTVEVLRQQPPDLVEFIVEYFTRLREARA

SEQ ID NO: 81 - protein, 44 aa, synthetic construct
SHIQIPPGLTELLQGYTVEVLRQQPPDLVEFVVEYFTRLREARA

SEQ ID NO: 82 - protein, 44 aa, synthetic construct
SHIQEPPGLTELLQGYTVEVLRQQPPDLVEFAVDYFTRLREARA

SEQ ID NO: 83 - protein, 17 aa, synthetic construct
NIEYLAKQIVDNAIQQA

SEQ ID NO: 84 - protein, 17 aa, synthetic construct
QLEYLAKQIVDNAIQQA

SEQ ID NO: 85 - protein, 17 aa, synthetic construct
QVEYLAKQIVDNAIQQA

SEQ ID NO: 86 - protein, 17 aa, synthetic construct
QIDYLAKQIVDNAIQQA

SEQ ID NO: 87 - protein, 17 aa, synthetic construct
QIEFLAKQIVDNAIQQA

SEQ ID NO: 88 - protein, 17 aa, synthetic construct
QIETLAKQIVDNAIQQA

SEQ ID NO: 89 - protein, 17 aa, synthetic construct
QIESLAKQIVDNAIQQA

SEQ ID NO: 90 - protein, 17 aa, synthetic construct
QIEYIAKQIVDNAIQQA

SEQ ID NO: 91 - protein, 17 aa, synthetic construct
QIEYVAKQIVDNAIQQA

SEQ ID NO: 92 - protein, 17 aa, synthetic construct
QIEYLARQIVDNAIQQA

SEQ ID NO: 93 - protein, 17 aa, synthetic construct
QIEYLAKNIVDNAIQQA

SEQ ID NO: 94 - protein, 17 aa, synthetic construct
QIEYLAKQIVENAIQQA

SEQ ID NO: 95 - protein, 17 aa, synthetic construct
QIEYLAKQIVDQAIQQA

SEQ ID NO: 96 - protein, 17 aa, synthetic construct
QIEYLAKQIVDNAINQA

SEQ ID NO: 97 - protein, 17 aa, synthetic construct
QIEYLAKQIVDNAIQNA

SEQ ID NO: 98 - protein, 17 aa, synthetic construct
QIEYLAKQIVDNAIQQL

SEQ ID NO: 99 - protein, 17 aa, synthetic construct
QIEYLAKQIVDNAIQQI

SEQ ID NO: 100 - protein, 17 aa, synthetic construct
QIEYLAKQIVDNAIQQV

SEQ ID NO: 101 - protein, 17 aa, synthetic construct
QIEYVAKQIVDYAIHQA

SEQ ID NO: 102 - protein, 17 aa, synthetic construct
QIEYLAKQIVDYAIHQA

SEQ ID NO: 103 - protein, 17 aa, synthetic construct
QIEYKAKQIVDHAIHQA

SEQ ID NO: 104 - protein, 17 aa, synthetic construct
QIEYHAKQIVDHAIHQA

SEQ ID NO: 105 - protein, 17 aa, synthetic construct
QIEYVAKQIVDHAIHQA

SEQ ID NO: 106 - protein, 17 aa, synthetic construct
LIEEAASRIVDAVIEQV

SEQ ID NO: 107 - protein, 17 aa, synthetic construct
PLEYQAGLLVQNAIQQA

SEQ ID NO: 108 - protein, 25 aa, synthetic construct
KGADLIEEAASRIVDAVIEQVKAAG

SEQ ID NO: 109 - protein, 25 aa, synthetic construct
PEDAELVRLSKRLVENAVLKAVQQY

SEQ ID NO: 110 - protein, 5 aa, synthetic construct
GGGGS

SEQ ID NO: 111 - protein, 4 aa, synthetic construct
GGGS

SEQ ID NO: 112 - skipped

SEQ ID NO: 113 - protein, 5 aa, synthetic construct
GSGGS

SEQ ID NO: 114 - protein, 4 aa, synthetic construct
GGSG

SEQ ID NO: 115 - protein, 5 aa, synthetic construct
GGSGG

SEQ ID NO: 116 - protein, 5 aa, synthetic construct
GSGSG

SEQ ID NO: 117 - protein, 5 aa, synthetic construct
GSGGG

SEQ ID NO: 118 - protein, 5 aa, synthetic construct
GGGSG

SEQ ID NO: 119 - protein, 5 aa, synthetic construct
GSSSG

SEQ ID NO: 120 - protein, 23 aa, synthetic construct
SHIQIPPGLTELLQGYTVEVLRQ

SEQ ID NO: 121 - protein, 18 aa, synthetic construct
PLEYOAGLLVONAIQQAI

SEQ ID NO: 122 - protein, 18 aa, synthetic construct
LLIETASSLVKNAIOLSI

SEQ ID NO: 123 - protein, 18 aa, synthetic construct
LIEEAASRIVDAVIEQVK

SEQ ID NO: 124 - protein, 18 aa, synthetic construct
ALYQFADRFSELVISEAL

SEQ ID NO: 125 - protein, 17 aa, synthetic construct
LEQYANQLADQIIKEAT

SEQ ID NO: 126 - protein, 17 aa, synthetic construct
FEELAWKIAKMIWSDVF

SEQ ID NO: 127 - protein, 18 aa, synthetic construct
ELVRLSKRLVENAVLKAV

SEQ ID NO: 128 - protein, 18 aa, synthetic construct
TAEEVSARIVQVVTAEAV

SEQ ID NO: 129 - protein, 18 aa, synthetic construct
QIKQAAFOLISOVILEAT

SEQ ID NO: 130 - protein, 16 aa, synthetic construct
LAWKIAKMIVSDVMQQ

SEQ ID NO: 131 - protein, 17 aa, synthetic construct
QIEYLAKQIPDNAIQQA

SEQ ID NO: 132 - protein, 25 aa, synthetic construct
KGADLIEEAASRIVDAVIEQVKAAG

SEQ ID NO: 133 - protein, 25 aa, synthetic construct
KGADLIEEAASRIPDAPIEQVKAAG

SEQ ID NO: 134 - protein, 25 aa, synthetic construct
PEDAELVRLSKRLVENAVLKAVQQY

SEQ ID NO: 135 - protein, 25 aa, synthetic construct
PEDAELVRTSKRLVENAVLKAVQQY

SEQ ID NO: 136 - protein, 25 aa, synthetic construct
PEDAELVRLSKRLPENAVLKAVQQY

SEQ ID NO: 137 - protein, 25 aa, synthetic construct
PDDAELVRLSKRLVENAVLKAVQQY

SEQ ID NO: 138 - protein, 25 aa, synthetic construct
PEDAELVRLSKRLVENAVEKAVQQY

SEQ ID NO: 139 - protein, 25 aa, synthetic construct
EEGLDRNEEIKRAAFQIISQVISEA

SEQ ID NO: 140 - protein, 25 aa, synthetic construct
LVDDPLEYQAGLLVQNAIQQAIAEQ

SEQ ID NO: 141 - protein, 25 aa, synthetic construct
QYETLLIETASSLVKNAIQLSIEQL

SEQ ID NO: 142 - protein, 25 aa, synthetic construct
LEKQYQEQLEEEVAKVIVSMSIAFA

SEQ ID NO: 143 - protein, 25 aa, synthetic construct
NTDEAQEELAWKIAKMIVSDIMQQA

SEQ ID NO: 144 - protein, 25 aa, synthetic construct
VNLDKKAVLAEKIVAEAIEKAEREL

SEQ ID NO: 145 - protein, 25 aa, synthetic construct
NGILELETKSSKLVQNIIQTAVDQF

SEQ ID NO: 146 - protein, 25 aa, synthetic construct
TQDKNYEDELTQVALALVEDVINYA

SEQ ID NO: 147 - protein, 25 aa, synthetic construct
ETSAKDNINIEEAARFLVEKILVNH